SEQUENCE LISTING (45 sequences)

SEQ ID NO: 1 - protein, 206 aa, Severe acute respiratory syndrome coronavirus 2
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

SEQ ID NO: 2 - protein, 851 aa, Artificial Sequence
  Description of Artificial Sequence: Synthetic polypeptide
QSTIEEQAKTFLDKFNVEAEDLFYQSSLASWNYNTNITEENVQNMNNAGDKWSAFLKEQSTLAQMYPLQEIQQLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYATSSGGGGENLYFQSSGGGSGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 11 aa, Artificial Sequence
  Description of Artificial Sequence: Synthetic peptide
VTGYRLFEEIL

SEQ ID NO: 4 - protein, 158 aa, Artificial Sequence
  Description of Artificial Sequence: Synthetic polypeptide
VFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 5 - protein, 137 aa, Severe acute respiratory syndrome coronavirus 2
GLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGS

SEQ ID NO: 6 - protein, 214 aa, Severe acute respiratory syndrome coronavirus 2
GLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKK

SEQ ID NO: 7 - protein, 162 aa, Severe acute respiratory syndrome coronavirus 2
PRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 8 - protein, 597 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 9 - protein, 851 aa, Artificial Sequence
  Description of Artificial Sequence: Synthetic polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYATSSGGGGENLYFQSSGGGSGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 360 aa, Artificial Sequence
  Description of Artificial Sequence: Synthetic polypeptide
MRMQLLLLIALSLALVTNSSGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSGGGTSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSGHHHHHHHHHHHHGSGLNDIFEAQKIEWHEG

SEQ ID NO: 11 - protein, 213 aa, Artificial Sequence
  Description of Artificial Sequence: Synthetic polypeptide
MRMQLLLLIALSLALVTNSSGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSGGGTSGGGGSVTGYRLFEEILGGGSGHHHHHHHHHHHHGSGLNDIFEAQKIEWHEG

SEQ ID NO: 12 - protein, 417 aa, Artificial Sequence
  Description of Artificial Sequence: Synthetic polypeptide
MRMQLLLLIALSLALVTNSTSRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLGGGGSGGGGSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSGHHHHHHHHHHHH

SEQ ID NO: 13 - protein, 280 aa, Artificial Sequence
  Description of Artificial Sequence: Synthetic polypeptide
MRMQLLLLIALSLALVTNSTSRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLGGGGSGGGGSGGGGSGGGGSGGGGSVTGYRLFEEILGGGSGHHHHHHHHHHHH

SEQ ID NO: 14 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 15 - protein, 38 aa, Severe acute respiratory syndrome coronavirus 2
ATEGALNTPKDHIGTRNPANNAAIVLQLPQGTTLPKGF

SEQ ID NO: 16 - protein, 38 aa, Severe acute respiratory syndrome coronavirus 2
NNAAIVLQLPQGTTLPKGFYAEGSRGGSQASSRSSSRS

SEQ ID NO: 17 - protein, 38 aa, Severe acute respiratory syndrome coronavirus 2
MAGNGGDAALALLLLDRLNQLESKMSGKGQQQQGQTVT

SEQ ID NO: 18 - protein, 5 aa, Artificial Sequence
  Description of Artificial Sequence: Synthetic peptide
GGGGS

SEQ ID NO: 19 - protein, 25 aa, Artificial Sequence
  Description of Artificial Sequence: Synthetic peptide
GGGGSGGGGSGGGGSGGGGSGGGGS

SEQ ID NO: 20 - protein, 6 aa, Artificial Sequence
  Description of Artificial Sequence: Synthetic peptide
GSSGSS

SEQ ID NO: 21 - protein, 8 aa, Artificial Sequence
  Description of Artificial Sequence: Synthetic peptide
GGGGGGGG

SEQ ID NO: 22 - protein, 12 aa, Artificial Sequence
  Description of Artificial Sequence: Synthetic peptide
GSAGSAAGSGEF

SEQ ID NO: 23 - protein, 15 aa, Artificial Sequence
  Description of Artificial Sequence: Synthetic peptide
GGGGSGGGGSGGGGS

SEQ ID NO: 24 - protein, 239 aa, Artificial Sequence
  Description of Artificial Sequence: Synthetic polypeptide
MGWSCIILFLVATATGVHSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 466 aa, Artificial Sequence
  Description of Artificial Sequence: Synthetic polypeptide
MGWSCIILFLVATATGVHSQMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 233 aa, Artificial Sequence
  Description of Artificial Sequence: Synthetic polypeptide
MGWSCIILFLVATATGVHCAIRMTQSPSSLSASVGDRVTITCQASQDISNYLNWYQQKPGKAPKLLIYDASNLETGVPSRFSGSGSGTDFTFTISSLQPEDIATYYCQQYDNLPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 477 aa, Artificial Sequence
  Description of Artificial Sequence: Synthetic polypeptide
MGWSCIILFLVATATGVHSQVQLVQSGAEVKKPGASVKVSCKASGYTFTGYYMHWVRQAPGQGLEWMGWINPISGGTNYAQKFQGRVTMTRDTSISTAYMELSRLRSDDTAVYYCASPASRGYSGYDHGYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 236 aa, Artificial Sequence
  Description of Artificial Sequence: Synthetic polypeptide
MGWSCIILFLVATATGSWAQSALTQPPSASGSPGQSVTISCTGTSSDVGGYKYVSWYQQHPGKAPKLMIYEVSKRPSGVPDRFSGSKSGNTASLTVSGLQAEDEADYYCSSYEGSNNFVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 467 aa, Artificial Sequence
  Description of Artificial Sequence: Synthetic polypeptide
MGWSCIILFLVATATGVHSQVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARGEGWELPYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 233 aa, Artificial Sequence
  Description of Artificial Sequence: Synthetic polypeptide
MGWSCIILFLVATATGVHSDIQMTQSPSTLSASVGDRVTITCRASQSISNWLAWFQQKPGKAPKLLIYEASSLESGVPSRFSGSGSGTEFTLTISSLQPDDFATYYCQQYNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 468 aa, Artificial Sequence
  Description of Artificial Sequence: Synthetic polypeptide
MGWSCIILFLVATATGVHSQVQLVESGGGVVQPGRSLRLSCAASGFTFSSYAMHWVRQAPGKGLEWVAVIPFDGRNKYYADSVTGRFTISRDNSKNTLYLQMNSLRAEDTAVYYCASSSGYLFHSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 233 aa, Artificial Sequence; identical to SEQ ID NO: 30
  Description of Artificial Sequence: Synthetic polypeptide

SEQ ID NO: 33 - protein, 246 aa, Artificial Sequence
  Description of Artificial Sequence: Synthetic polypeptide
MGWSCIILFLVATATGVHSQVQLVESGGGVVQPGRSLRLSCAASGFTFSSYAMHWVRQAPGKGLEWVAVIPFDGRNKYYADSVTGRFTISRDNSKNTLYLQMNSLRAEDTAVYYCASSSGYLFHSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 34 - protein, 10 aa, Artificial Sequence
  Description of Artificial Sequence: Synthetic peptide
GGGTSGGGGS

SEQ ID NO: 35 - protein, 442 aa, Artificial Sequence
  Description of Artificial Sequence: Synthetic polypeptide
MRMQLLLLIALSLALVTNSSGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKGGGGSGGGTSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSGHHHHHHHHHHHHGSGLNDIFEAQKIEWHEG

SEQ ID NO: 36 - protein, 295 aa, Artificial Sequence
  Description of Artificial Sequence: Synthetic polypeptide
MRMQLLLLIALSLALVTNSSGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKGGGGSGGGTSGGGGSVTGYRLFEEILGGGSGHHHHHHHHHHHHGSGLNDIFEAQKIEWHEG

SEQ ID NO: 37 - protein, 261 aa, Artificial Sequence
  Description of Artificial Sequence: Synthetic polypeptide
TSRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLGGGGSGGGGSGGGGSGGGGSGGGGSVTGYRLFEEILGGGSGHHHHHHHHHHHH

SEQ ID NO: 38 - protein, 398 aa, Artificial Sequence
  Description of Artificial Sequence: Synthetic polypeptide
TSRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLGGGGSGGGGSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSGHHHHHHHHHHHH

SEQ ID NO: 39 - protein, 261 aa, Artificial Sequence
  Description of Artificial Sequence: Synthetic polypeptide
TSRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLGGGGSGGGGSGGGGSGGGGSGGGGSVTGYRLFEEILGGGSGHHHHHHHHHHHH

SEQ ID NO: 40 - protein, 398 aa, Artificial Sequence
  Description of Artificial Sequence: Synthetic polypeptide
TSRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLGGGGSGGGGSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSGHHHHHHHHHHHH

SEQ ID NO: 41 - protein, 329 aa, Artificial Sequence
  Description of Artificial Sequence: Synthetic polypeptide
TVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGGSGGGGSGGGGSGGGGSGGGGSVTGYRLFEEILGGGSGHHHHHHHHHHHH

SEQ ID NO: 42 - protein, 466 aa, Artificial Sequence
  Description of Artificial Sequence: Synthetic polypeptide
TVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGGSGGGGSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSGHHHHHHHHHHHH

SEQ ID NO: 43 - protein, 276 aa, Artificial Sequence
  Description of Artificial Sequence: Synthetic polypeptide
TVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVD

SEQ ID NO: 44 - protein, 208 aa, Artificial Sequence
  Description of Artificial Sequence: Synthetic polypeptide
TSRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNL

SEQ ID NO: 45 - protein, 208 aa, Artificial Sequence
  Description of Artificial Sequence: Synthetic polypeptide
TSRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNL

(19) United States
(12) Patent Application Publication
(10) Pub. No.: US 2023/0296621 A1
(43) Pub. Date: Sep. 21, 2023

(54) DETECTION ASSAY FOR ANTI-SARS-COV-2 ANTIBODIES

(71) Applicants: Chan Zuckerberg Biohub, Inc., San Francisco, CA (US); The Regents of the University of California, Oakland, CA (US)

(72) Inventors: James A. Wells, Oakland, CA (US); Susanna Elledge, Oakland, CA (US); Xin Zhou, Oakland, CA (US)

(21) Appl. No.: 17/997,878

(22) PCT Filed: May 11, 2021
(86) PCT No.: PCT/US2021/031871

§ 371 Date: Nov. 3, 2022

Related U.S. Application Data
(60) Provisional application No. 63/067,273, filed on Aug. 18, 2020.
(60) Provisional application No. 63/058,379, filed on Jul. 29, 2020.
(60) Provisional application No. 63/056,509, filed on Jul. 24, 2020.
(60) Provisional application No. 63/022,789, filed on May 11, 2020.

(51) Int. Cl. G01N 33/68; C12N 9/02

(52) U.S. Cl. CPC G01N 33/6854; C12N 9/0069; C12Y 113/12007; G01N 2469/20; G01N 2470/10

(57) ABSTRACT

Protein biosensors and methods of using these sensors to detect anti-SARS-CoV-2 patient antibodies (Abs) in a solution-based, rapid, and quantitative COVID-19 serological assay are provided. In certain aspects, the sensors each comprise a first fusion protein that comprises a first SARS-CoV-2 viral protein and a first peptide fragment of a split reporter protein, and a second fusion protein that comprises a second fusion protein that comprises a second SARS-CoV-2 viral protein domain and a second peptide fragment of the split reporter protein. Only if the test sample comprise SARS-CoV- antibodies, the first and second peptide fragments associate to produce an enzymatically active reporter protein.

### CROSS REFERENCE TO RELATED APPLICATIONS

This application claims priority to U.S. Provisional Application No. 63/022,789, filed on May 11, 2020; U.S. Provisional Application No. 63/056,509, filed on Jul. 24, 2020; U.S. Provisional Application No. 63/058,379, filed on Jul. 29, 2020; and U.S. Provisional Application No. 63/067,273, filed on Aug. 18, 2020. The entire disclosure of each of the aforementioned provisional applications is herein incorporated by reference for all purposes.

### SEQUENCE LISTING

The instant application contains a Sequence Listing which has been submitted electronically in ASCII format and is hereby incorporated by reference in its entirety. Said ASCII copy, created on May 11, 2021, is named 103182-1244658-005510WO_SL.txt and is 108,132 bytes in size.

### BACKGROUND

COVID-19, caused by the SARS-CoV-2 virus, has spread throughout the world and, as of July 2020, has resulted in close to 4 million cases and over 270,000 deaths globally. Early detection of disease using viral detection assays is critical for containing the spread of this virus. Clinical laboratory tests and point-of-care tests are needed for screening and diagnosis of infected individuals. The most widely used tests currently are PCR based tests that detect viral RNA in patient samples. See Esbin et al., 2020, “Overcoming the bottleneck to widespread testing: A rapid review of nucleic acid testing approaches for COVID-19 detection” RNA doi:10.1261/rna.076232.120. However, these methods require viral RNA extraction, reverse transcription PCR, and quantitative PCR reactions, which limit the throughput of the assay, require expensive equipment and reagents, and takes hours or days to produce results.

Serological assays are vital tools for monitoring how a patient’s anti-viral immune response evolves after the period of acute infection ends. The results of these assays, which detect antibodies (e.g. IgG, IgM) against viral antigens in patient serum, are also important to aid diagnostics, identify donors for convalescent serum therapeutic, support vaccine and therapeutic development, and inform epidemiologic studies.

### BRIEF DESCRIPTION OF THE DRAWINGS

FIG. 1A shows a schematic of the solution based serology assay. Patient antibodies are incubated with SARS-CoV-2 SpikeRBD or N protein sequences fused to LgBiT/SmBiT. For the population of antibodies with one arm bound to the LgBiT sensor and the other arm bound to the SmBiT sensor, the nanoBiT luciferase enzyme is reconstituted to form an active luciferase, which produces signal.

FIG. 1B is a schematic representation of the SARS-CoV-2 Spike protein. The SpikeRBD domain consists of amino acids 328 - 533.

FIG. 1C shows the results of the protein yield of the five (5) S sensors, i.e., SpikeRBD-NanoBiT sensor fusions. Each of the SpikeRBD LgBiT sensors comprises a 5aa, 15aa, or a 25aa GS linker between the LgBiT and the Spike RBD. Each of the SpikeRBD SmBiT sensors comprises a 15aa GS linker or a 25aa GS linker.

FIG. 1D shows the results of detecting CR3022 using S sensors at various concentrations of sensors and CR3022. The results show that the S sensors are most sensitive at the concentration of 1 nM for detecting CR3022 in solution compared to S sensors that were used at higher or lower concentrations, i.e., 0.11 nM, 0.33 nM, 3 nM, 9 nM, and 27 nM. The results show that the S sensors were most sensitive at 1 nM for detecting CR3022.

FIG. 1E shows that patient antibodies for SARS-CoV-2 recognize various epitopes on the SpikeRBD. C004 and C105 have ACE2 competitive epitopes, while C135 and CR3022 have non-ACE2 competitive epitopes.

FIG. 1F shows that the S sensors can detect patient antibodies of various epitopes with similar sensitivity. C004, C105, C135, and CR3022, which are patient antibodies that are known to bind to Spike protein, were incubated with the S sensors at 10 fold dilutions. The concentrations of these antibodies range from 10 nM to 0.001 nM of antibody. FIGS. 1G-1H (reserved)

FIG. 1I shows an annotated schematic representation of the SARS-CoV-2 Nucleocapsid protein (N protein). All N protein sensors disclosed in this application include the RNA binding domain (aa 44-180, SEQ ID NO: 5) and exclude the dimerization domain (aa 257-419, SEQ ID NO: 7. The full length N Protein sequence is

MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA (SEQ ID NO:14).

FIG. 1J shows the results of expressing two different N protein fusions: a C terminal fusion (aa 44-180 N protein - 10aa linker - LgBiT/SmBit) and an N terminal fusion (LgBiT/SmBiT - 10aa linker - aa 44-257 N protein).

FIG. 2A shows the results of using the S sensors to detect antibodies in five (5) COVID-19 recovered patients. Each serum sample was diluted 12.5 fold, 25 fold, and 50 fold. At all dilutions, serum samples from all 4 patients generated signal above the background signal of two control serum samples collected before the pandemic. The data points represent the average of duplicates from two separate experiments. The error bars represent the standard deviation.

FIGS. 2B and 2C show the results of using the N sensors to detect antibodies in four (4) COVID-19 recovered patients (patients 6, 8, 11, and 12) and two controls. Each serum sample was diluted 12.5 fold, 25 fold, and 50 fold. At all dilutions, serum samples from all 4 patients generated signal above the background signal of two control serum samples collected before the pandemic. These data points represent the average of duplicates from one experiment. The error bars represent the standard deviation.

FIG. 2D shows a solution serology assay tested on expanded COVID-19 recovered outpatient cohort and control samples. These were all outpatient serum samples from individuals that have been free of COVID-19 symptoms for 14+ days. 48/57 of the patients showed signal for the S sensors above the control level (dashed line, S cutoff). 57/57 of the patients showed signal for the N sensors above the control level (dashed line, N cutoff). Bars represent the average between two technical duplicates.

FIG. 2E shows results of testing patient samples and control seasonal coronavirus/flu samples using the methods disclosed herein. For the S sensors, 49 out of 55 (49/55) patients showed detectable anti-S antibody levels above control samples (dashed line, S cutoff). For the N sensors, 41/55 patients showed detected anti-N antibody levels above the control samples (dashed line, N cutoff). Seasonal coronavirus and flu vaccine controls (56-87) did not show greatly elevated signal, although we did notice slightly higher background with the seasonal coronavirus samples (56-77) compared to the flu vaccine control samples for the N protein sensors.

FIG. 2F shows that signal from the S sensor assay correlates well with S protein-based ELISA signal for the outpatient sample cohort. The correlation is strong to the anti-Fab and anti-IgG ELISA signal. There is no significant correlation to the anti-IgM signal.

FIG. 2G shows a positive correlation (R=0.47) between S sensor signal and N sensor signal in outpatient serum samples.

FIG. 2H shows the correlation of the signal from the solution serology assay using the protein sensors disclosed herein to neutralization capability. S sensor signal (round dots) and N sensor signal (square dots) is plotted against 50% maximal neutralization titer (NT50). Both show positive correlation, but there is a stronger correlation between NT50 and S sensor signal (R=0.72) than N sensor signal (R=0.47).

FIG. 2I is a schematic representation of a competition assay to determine proportions of antibodies present in the serum sample that share an epitope with a recombinant Fab against SpikeRBD. The S sensors are pre-incubated with the Fab before adding to the patient antibody for detection. This blocks all antibodies that share an epitope with the Fab from binding to the sensors and thus reduce signal generated from those antibodies.

FIG. 2J shows the results of a competition epitope assay with a neutralizing patient antibody C135, which binds to an epitope different from ACE2, CR3022, C004 or C105. Twelve of the patients from the outpatient cohort with incubated with either no Fab or C135 Fab. Patient 72 (source of the C135 antibody) had a decrease in signal in the presence of the C135 Fab. In addition to Patient 72, Patient 7, 21, 42, 98, 202 also had a decrease in signal with C135, indicating the prevalence of antibodies competing with this antibody. The decreases in signal are more modest compared to the control recombinant C135 IgG due to the presence of many antibodies in a patient sample of various epitopes. Bars represent the average of two replicates, error bars represent standard deviation.

FIGS. 2K-2L show proper negative (FIG. 2K) and positive control conditions (FIG. 2L). FIG. 2K shows adding 4-10% v/v FBS to PBST (PBS + 0.05% Tween-20) reduces background to a level close to background observed in serum experiments as described in FIG. 2B above. FIG. 2L shows recombinant patient antibody C004 generates linear dose-dependent signal from 0.1-10 nM from 0.1 to 10 nM in the PBST + 10 v/v % FBS buffer. Thus, C004 or other SARS-CoV-2 recombinant antibodies can serve as positive controls in assays.

FIGS. 3A-3B show that although the overall signal is lower, the lyophilized S and N sensors detected patient antibodies from blood samples. The data points represent the average of two technical duplicates. The error bars represent the standard deviation. The dotted line represents the cutoff. S sensor showed seropositivity for 4/6 patients, and N sensor showed seropositivity for 6/6 patients.

FIGS. 3C-3E show S and N sensors remain functional after lyophilization. The FIG. 3C shows the percent protein recovery and percent signal after the lyophilization process comparing to fresh sensors. FIG. 3D shows although the overall signal is slightly lower, the lyophilized S sensor provided similar detection sensitivity of CR3022 IgG. FIG. 3E shows lyophilized N sensor provided very similar signal in the serology assay with two patient samples.

FIGS. 4A and 4B show the results of computer modeling of CR3022 binding to SpikeRBD-SmBiT/LgBiT sensors (FIG. 4A) and an ACE2-competitive Ab binding to SpikeRBD-SmBiT/LgBiT sensors (FIG. 4B). The measured distances: 43.5, 41.5, 55.2 and 57.1 angstrom guided the design of the linker lengths.

FIG. 5A shows the results of linker variants characterizations with CR3022. S sensors with varied linker lengths resulted in similar sensitivity in detecting CR3022. FIG. 5B shows that an ACE2-Fc variant that has a higher affinity to SpikeRBD generated a higher signal compared to the wild type ACE2-Fc.

FIG. 6 (part A) is a schematic illustration of the Spike-RBD-LgBiT and Spike-RBD-SmBiT-based serology assay to detect anti-Spike-RBD patient antibodies. FIG. 6 (parts B-D) show the proof of concept results that the Spike-RBD NanoBiT system is able to detect various concentrations of recombinant anti-Spike-RBD antibodies or ACE2-Fc protein.

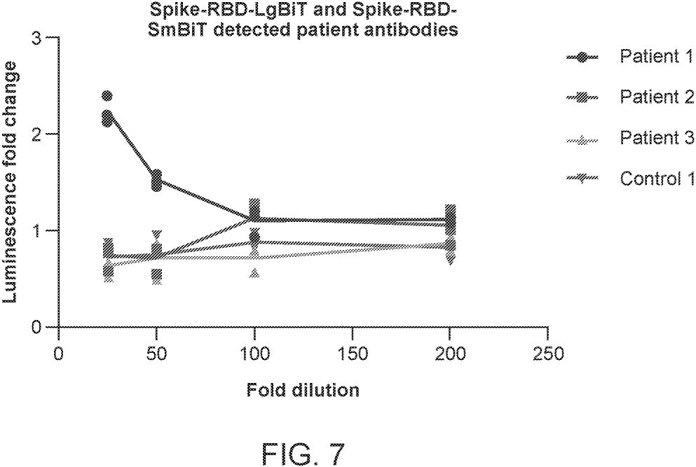

FIG. 7 shows the results of the serology assay when using the Spike-RBD NanoBiT system with patient samples.

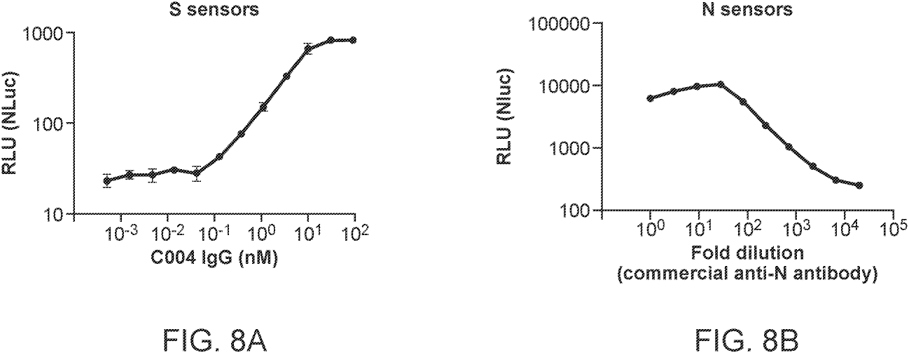

FIG. 8A shows the dose-dependent signal from the split luciferase antibody biosensor (spLUC) test for the recombinant anti-S-RBD antibody C004 in PBST + 10% FBS.

FIG. 8B shows dose-dependent spLUC signals for an anti-N-RBD antibody (Sino Biological, Cat#40588-T62-50) in PBST+ 8% FBS.

FIGS. 9A-9I show the results of characterization of outpatient and inpatient serum samples using the spLUC test. Cohort 1: samples drawn during the convalescent phase of an outpatient group, Cohort 2: samples drawn during the acute phase or the convalescent phase of a hospitalized group, and Cohort 3: samples drawn during the convalescent phase of a mixed inpatient and outpatient group. A 10-base logarithmic scale conversion was applied to all the solution assay signals for the correlation analysis unless otherwise specified. (A) SpLUC assay tested on expanded COVID-19 patient cohorts with S sensors at 1:12.5 serum dilution. Dots represent the average between two technical duplicates. Lines represent median values. The inpatient samples showed significantly higher antibody titers than the outpatient cohorts. (B) SpLUC assay tested on expanded COVID-19 patient cohorts with N sensors at 1:12.5 serum dilution. The inpatient samples showed significantly higher antibody titers than the outpatient cohorts. (C) A positive correlation (R = 0.78) was observed between S sensor signal and N sensor signal in the three cohort samples. All cohorts individually presented a similar trend (FIG. S8). Line represent linear regression. (D) Correlation of spLUC signals (cohort 1) to neutralization efficiency (Robbiani et al., 2020). S sensor signal (blue) and N sensor signal (purple) is plotted against 50% maximal neutralization titer (NT50). Both show positive correlation (R = 0.76 for S and NT50 259 and R = 0.62 for N and NT50). (E) Inpatients show significantly higher signal over outpatients in all three cohorts (p < 0.0001). (F) Patients from cohort 1 that reported higher disease severity (6-10 vs 1-5) had higher antibody titer for both S and N sensors and the difference for N sensors is statistically significant (p = 0.0049). g, Higher overall antibodies titers were observed in patients that reported fever compared to no fever patients for cohort 3. Lines represent median values. This difference was statistically significant for the S sensors (p = 0.0011) but not N sensors. (H) Slightly higher overall antibodies titers were observed in females compared to males for cohort 3, although the differences were not statistically significant. There is a similar trend for cohort 1 (FIG. 9A). The difference was more obvious for S sensors. Lines represent median values. (I) For cohort 3, there is a slightly higher level of antibodies in the 60-85 age group compared to 19-39 and 40-59. There is a similar trend for cohort 1 (FIG. 9B). The differences were not statistically significant. Lines represent median values. For FIGS. 9A, 9B and 9F-9I, the Mann-Whitney test P values for each comparison are labeled on top of the datasets. For c-d, the Spearman R values and P values are labeled in the graphs. For all figures, dots represent the average of two technical replicates. Horizonal lines represent median values. For c, d, lines represent linear regression.

FIG. 10 shows that Signals from the S sensor spLUC assay (cohort 2) correlate very well with SpikeRBD ELISA anti-Fab signals (R = 0.84) and with anti-IgG signals (R = 0.86), but poorly with anti-IgM signals for cohort 1 (R = 0.29).

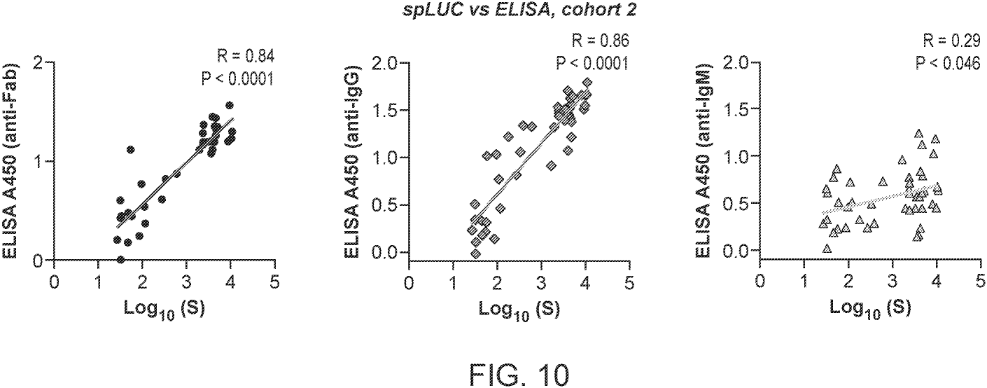

FIG. 11 shows that spLUC reactions are compatible with saliva samples. The CR3022 antibody was spiked into healthy individual saliva at 10-fold dilutions from 100 nM to 0.01 nM. While undiluted saliva reduced signal 10-fold and reduced sensitivity, 1:2 dilution of saliva only reduced signal by 3-fold and did not decrease the sensitivity. Each dot represents the average of two technical replicates and error bars represent standard deviation.

FIG. 12 shows a strong correlation of the assay signals with the original Spike-RBD sensor (X axis) and the assay signals with the Spike-RBD variant sensors and a new Spike-NTD sensor (Y axis) for convalescent SARS-CoV-2 patient samples.

### SUMMARY OF INVENTION

In one aspect, disclosed herein is a method for detecting antibodies against a SARS-CoV-2 viral protein in a biological sample. The method includes combining a) the biological sample; b) a first fusion protein that may include a first SARS-CoV-2 viral protein domain and a first peptide fragment of a split reporter protein, and c) a second fusion protein that may include a second SARS-CoV-2 viral protein domain and a second peptide fragment of the split reporter protein to produce a mixture. The method further comprises maintaining the mixture under conditions in which, only if the test sample may include individual antibodies, at least one of which binds the first and the second SARS-CoV-2 viral protein domain simultaneously, the first peptide fragment and the second peptide fragment associate to produce an enzymatically active reporter protein. The method further comprises detecting the association of the first peptide fragment and the second peptide fragment if the test sample may include antibodies against the SARS-CoV-2 viral protein.

In one aspect, disclosed herein is a method for detecting antibodies against a SARS-CoV-2 viral protein in a patient sample, wherein the viral protein is the N protein, wherein the method comprises: i) preparing a mixture comprising the patient sample; a first fusion protein that comprises a first SARS-CoV-2 viral protein domain and a first peptide fragment of a split reporter protein, and a second fusion protein that comprises a second SARS-CoV-2 viral protein domain and a second peptide fragment of the split reporter protein; ii) maintaining the mixture under conditions in which, only if the test sample comprises individual antibodies, at least one of which binds the first and second SARS-CoV-2 viral protein domains, the first peptide fragment and the second peptide fragment associate to produce a detectable reporter protein; wherein each of the first SARS-CoV-2 viral protein domain and the second SARS-CoV-2 viral protein domain comprise a sequence that is at least 90% identical to SEQ ID NO: 5, and iii) detecting the association of the first peptide fragment and the second peptide fragment if the patient sample comprises antibodies against the SARS-Cov-2 N protein.

In one aspect, disclosed herein is a method for detecting antibodies against a SARS-CoV-2 viral protein in a patient sample. The method also includes i) preparing a mixture may include. The method also includes a) the patient sample; b) a first fusion protein that may include a first SARS-CoV-2 viral protein domain and a first peptide fragment of a split reporter protein, and c) a second fusion protein that may include a second SARS-CoV-2 viral protein domain and a second peptide fragment of the split reporter protein. The method also includes ii) maintaining the mixture under conditions in which, only if the test sample may include individual antibodies, at least one of which binds the first and second SARS-CoV-2 viral protein domains, the first peptide fragment and the second peptide fragment associate to produce a detectable reporter protein; where each of the first SARS-CoV-2 viral protein domain and the second SARS-CoV-2 viral protein domain may include a sequence that is at least 90% identical to seq id no: 5, and iii) detecting the association of the first peptide fragment and the second peptide fragment if the patient sample may include antibodies against the SARS-CoV-2 N protein.

In some embodiments, the first and second SARS-CoV-2 viral protein domains are the same. In some embodiments, the antibodies are detected are neutralizing antibodies, i.e. can neuturalize infection of SARS-CoV-2. In some embodiments, the split reporter protein is a luciferase. In some embodiments, the first protein peptide fragment comprise a sequence of SEQ ID NO: 4 (LgBiT) and second protein peptide fragment comprises a sequence of SEQ ID NO: 3 (SmBiT). In some embodiments, the first peptide fragment is fused to the C-terminus of the N protein domain, and the second peptide fragment is fused to the C-terminus of the second N protein domain. In some embodiments, the first peptide fragment is fused to the first SARS-CoV-2 viral protein domain via a first flexible linker and/or the second peptide fragment is fused to the second SARS-CoV-2 viral protein domain via a second flexible linker. Each of the first and second flexible linkers may have a length in the range of one to 50 amino acids.

In some embodiments, each of the first SpikeRBD domain and the second SpikeRBD comprises a sequence that is at least 90% identical to SEQ ID NO: 1, wherein the first flexible linker has a length of 15 amino acids and the second flexible linker has a length of 25 amino acids. In some embodiments, each of the N protein domain and the second N protein domain comprises a sequence that is at least 90% identical to SEQ ID NO: 5, wherein the first flexible linker and the second flexible linker has a length of 10 amino acids. In some embodiments, the first fusion protein is present in the mixture at a concentration in the range from 0.3 nM to 10 nM, and/or the second fusion protein is present in the mixture at a concentration in the range from 0.3 nM to 10 nM. In some embodiments, the first fusion protein and the second fusion protein are present in the mixture at about equal molar concentration.

In one aspect, disclosed herein is a kit for detecting antibodies against a SARS-CoV-2 spike protein in a biological sample. The kit also includes i) a first fusion protein that comprises a first SARS-CoV-2 viral protein domain and a first peptide fragment of a split reporter protein, and ii) a second fusion protein that comprises a second SARS-CoV-2 viral protein domain and a second peptide fragment of the split reporter protein, wherein the first SARS-CoV-2 viral protein domain shares at least 90% sequence identity with the second viral protein domain, wherein, only if the test sample comprises individual antibodies, at least one of which binds both of the first and second SARS-CoV-2 viral protein domains simultaneously, the first peptide fragment and the second peptide fragment associate to produce a detectable reporter protein.

In some embodiments, disclosed herein is a kit for detecting antibodies against a SARS-Cov-2 N protein in a biological sample, wherein the kit comprises: i) a first fusion protein that comprises a first N protein domain and a first peptide fragment of a split reporter protein, and ii) a second fusion protein that comprises a second N protein domain and a second peptide fragment of the split reporter protein, wherein each of the first N protein domain and the second N protein domain comprises a sequence that is at least 90% identical to SEQ ID NO: 5, wherein, only if the biological sample comprises individual antibodies, at least one of which binds both of the first and second SpikeRBD domains simultaneously, the first peptide fragment and the second peptide fragment associate to produce a detectable reporter protein.

In some embodiments, disclosed herein is a kit for detecting antibodies against a SARS-Cov-2 Spike protein in a biological sample, wherein the kit comprises: i) a first fusion protein that comprises a first SpikeRBD domain and a first peptide fragment of a split reporter protein, and, ii) a second fusion protein that comprises a second SpikeRBD domain and a second peptide fragment of the split reporter protein, wherein each of the first and the second SpikeRBD domains comprise a sequence that is at least 90% identical to SEQ ID NO: 1, wherein, only if the test sample comprises individual antibodies, at least one of which binds both of the first and second SpikeRBD domains simultaneously, the first peptide fragment and the second peptide fragment associate to produce a detectable reporter protein.

In some embodiments, the first peptide fragment is fused to the C- terminus of the first N protein domain, and the second peptide fragment is fused to the C- terminus of the second N protein domain. In some embodiment, the kit further comprises a substrate for the split-luciferase In some embodiments, the split reporter protein is a split-luciferase. In some embodiments, one or more of the first fusion protein, the second fusion protein, dilution buffer, substrate for reporter protein (e.g., luciferase) are lyophilized. In some embodiments, the first peptide fragment comprises a sequence of SEQ ID NO: 4 and the second peptide fragment comprise a sequence of SEQ ID NO: 3. In some embodiments, the kit further comprises a negative control sample, wherein the negative control sample comprises PBST and 4-10% PBS. In some embodiments, the kit further comprises a positive control sample, wherein the positive control sample comprises an antibody known to specifically bind to the SARS-CoV-2 Spike RBD domain or to an ACE-Fc protein. In some embodiments, the kit further comprises a positive control sample, wherein the positive control sample comprises a known antibody that is against the N protein.

In yet another aspect, provided herein is a reaction mixture comprising i) a test sample, ii) a first fusion protein that comprises a first SARS-CoV-2 viral protein domain of a viral protein of SARS-CoV-2 and a first peptide fragment of a split reporter protein, and iii) a second fusion protein that comprises a second SARS-CoV-2 viral protein domain of the viral protein of SARS-CoV-2 and a second peptide fragment of the split reporter protein, wherein only if the test sample comprises antibodies that bind the first and second SARS-CoV-2 viral protein domains, the first peptide fragment and the second peptide fragment associate to produce a detectable reporter protein.

In some embodiments, the viral protein is the S protein, and wherein each of the first viral protein domain and the second viral protein domain comprises a sequence that is at least 90% identical to SEQ ID NO: 1. In some embodiments, the viral protein is the N protein, and wherein each of the first viral protein and the second viral protein comprise a sequence that is at least 90% identical to SEQ ID NO: 5.

In another aspect, provided herein is a method of determining if antibodies in a test sample is competitive with a reference antibody against a viral protein domain of SARS-CoV-2. The method comprises: i) contacting a viral protein sensor, wherein the viral protein sensor comprises the first and second fusion proteins according to claim 1 with a first aliquot of the test sample, and detecting a first signal produced from association of the first peptide fragment and the second peptide fragment, ii) contacting an epitope-masked viral protein sensor with a second aliquot of the test sample, and detecting a second signal produced from association of the first peptide fragment and the second peptide fragment in the epitope-masked sensor, wherein the epitope-masked viral protein sensor comprises the viral protein sensor that is bound to a reference antibody, wherein the reference antibody is capable of binding to the SARS-CoV-2 viral protein domain at a known epitope; and iii) determining that the test sample comprises antibodies competitive with the reference antibody if the first signal is substantially higher than the second signal.

### DETAILED DESCRIPTION

### 1. Overview

In one aspect the invention is related to protein biosensors used to detect anti-SARS-CoV-2 patient antibodies (Abs) detection in a solution-based, rapid, and quantitative COVID-19 serological assay, also referred to as the split luciferase antibody biosensor (spLUC) test. This section describes certain general feature of protein biosensors, but is not intended to be limiting or comprehensive.

A “protein biosensor,” as used herein, may refer to a pair of fusion proteins (which may be called a cognate pair) that can be used together to detect antibodies against a SARS-CoV-2 antigen. Each fusion protein of the pair comprises at least two domains: A viral protein domain (V) and a detection moiety domain (D), where the detection moieties of the two members of the pair are complementary portions of a split reporter. As used in this context, “complementary” means that, when in proximity, the detection moieties (optionally with other components) may combine to generate a detectable complex. For example, when combined the detection moieties may form a complex with a luciferase activity not found in either individual moiety. In some embodiments the V and D domains are connected by a peptide linker domain (L). Thus, for illustration and not limitation in some embodiments each member of a construct pair is a fusion protein with the structure V-D or V-L-D. Optionally additional sequences may be found (e.g., amino-terminal to D). When the two members of a construct pair are bound by an antibody against the viral protein domain(s) the detection moiety domains are brought into proximity and associate to form an active reporter. In this disclosure, when the reporter is a protein, a detection moiety domain (D) may be alternatively referred to as a “peptide fragment.” A signal generated from the active reporter protein can be quantified to indicate the presence of the antibody. For example, a sample can be determined as comprising SARS-CoV2 antibodies that can bind to the protein sensor if a detected signal from the sample is above a cutoff (aka. a cutoff value); and conversely, a sample can be determined as not comprising detectable levels of anti-SARS-CoV-2 antibodies if the detectable signal is equal to or below the cutoff. Methods for determining cutoffs are disclosed herein.

For convenience, the two fusion protein members of a cognate pair can be referred to as alpha (α) and beta (β). For illustration and not for limitation, the structure of a first member can be described as αV-αD or αV-αL-αD and the structure of the second member can be described as βV-βD or βV-βL-βD. As noted, αD and βD are complementary portions of a split reporter.

Naturally occurring antibodies, such as antibodies directed a SARS protein, generally have two (e.g., IgG) or more (e.g., IgM) identical antigen binding pockets. The antigen binding sites recognize the same epitope. However, a single antibody may bind two different epitopes. For example, an antibody may be cross-reactive. Likewise, the epitopes presented by two different polypeptides with non-identical sequences may have sufficient similarity to be recognized by the same bivalent antibody. In many embodiments the viral protein domains αV and βV are the same (i.e., have identical amino acid sequences) reflecting that each presents the same viral epitope which is recognized by a bivalent patient antibody. However, it is contemplated that in some embodiments αV and βV may have different amino acid sequences, provided each amino acid sequence is bound by the same single antibody. Typically, αV and βV, if not identical, will have similar sequences.

The linker moieties, αL and βL may be the same or different (e.g., the αL and βL may be of different lengths or sequences). Both, only one, or neither of αL and βL may be present in the fusion protein pair.

As discussed in detail below, in one aspect the of the invention, the viral protein domains of a protein biosensor are from the SARS-CoV-2 nucleocapsid (N) protein or from the RBD portion of the SARS-CoV-2 Spike (S) protein.

The methods disclosed herein are simple to perform (typically involving no wash steps between reagent addition steps), and they are sensitive (e.g., the sensitivity can be greater than or equal to 98%), specific (greater than or equal to 99%), fast (can be as short as 5 minutes), and require only a small sample volume (e.g., 1 µl per reaction).

It will be recognized by the skilled reader that, although a protein biosensor that detects SARS-CoV-2 N or S proteins, the invention may be adapted for detecting antibodies against other proteins from the SARS-CoV-2 as well as broad range of other viral or bacterial antigens associated with other infectious diseases.

### 2. Subjects and Patient Samples

In some aspects, the protein sensors described herein are used in an solution-based assay to detect antibodies. In one aspect the protein sensors and methods described herein are used to detect antibodies against a SARS-CoV-2 antigen and determine whether a patient is and/or has been infected with SARS-CoV-2 and generated an immune response against the virus. Clinical or screening assays are carried out using an antibody-containing biological sample obtained from a subject.

### 2.1 Subjects

The subject can be tested using the methods disclosed herein may be a mammal, e.g., a human. The subject may be male or female and may be a juvenile or an adult (e.g., at least 30 years old, at least 40 years old, or at least 50 years old.)

The subject may be symptomatic or asymptomatic. In some cases, the subject has been positively diagnosed as having a SARS-CoV-2 infection. In some cases, the subject exhibits symptoms consistent with a SARS-CoV-2 infection (e.g., one or more of fever, fatigue, cough, myalgia, nausea or vomiting, shortness of breath, headache, and loss of smell or taste). In some cases, the subject may not manifest any symptoms that are typically associated with the SARS-CoV-2 infection. In some cases the subject is known or believed to have been exposed to SARS-CoV-2, suspected of having exposure to SARS-CoV-2, or believed not to have had exposure to SARS-CoV-2. In some cases, the subject may have recovered from a prior exposure of SARS-CoV-2 (also referrend to as a convalescent patient). In some cases, the subject has received a SARS-CoV-2 vaccine. The SARS-CoV-2 vaccine can be any of the DNA, RNA, or protein, or inactive SARS-CoV-2 virus that is capable of inducing immune response in a patient to generate anti SARS-CoV-2 antibodies. In some cases, the subject has been free of symptoms suggestive of a SARS-CoV-2 infection for at least 14 days. In some cases, the subject may have one or more of other conditions of hypertension, coronary artery disease, diabetes, chronic obstructive pulmonary disease.

### 2.2 Samples

In some cases, the sample tested (“test sample”) using the methods disclosed herein is a biological sample, which may be obtained from a human or other mammals. Typically a biological sample obtained from a human subject is referred to as a patient sample. The word “patient,” in this context does not connote that the subject is ill, infected, recovering from infection, or previously infected. A biological sample may be obtained from a tissue of a subject or bodily fluid isolated from a subject. Typically the sample is whole blood or a blood product (such as plasma, serum, whole blood, dried blood, settled blood, pooled blood, blood cells, or a blood product intended for transfusion or treatment), lymph fluid or saliva. Other sources include, without limitation, sputum, synovial fluid, urine, tears, organs, tissues, veterinary samples, environmental samples, and food samples.

### 3. Protein Sensors

The terms “protein biosensor,” “sensor,” “biosensor” and the like are used interchangeably. Based, in part, on the discoveries described in the Examples and discussed below, we have developed proximity-based binding assays for the detection of antibodies against a viral protein of the SARS-CoV-2. A proximity assay (or proximity-based binding assay) produces a detectable signal when two binding events occur physically close to each other and at the same time. Examples of proximity assays include split reporter-type assays, proximity ligation, and proximity extension assays.

In some aspects, the assay involves combining a portion of the test sample (e.g., serum) with a protein sensor, e.g., the S Sensor or the N Sensor, under conditions in which antibody-antigen binding occurs if anti-virus antibodies are present in the sample (“assay conditions”). As discussed in Section 1, in one aspect, a protein sensor includes a cognate pair of fusion proteins and each member of the pair comprises a viral protein domain (V) fused to a detection moiety domain (D), where the detection moieties of the two members are complementary portions of a split reporter. Stated differently, in one approach each protein sensor comprises a first fusion protein and a second fusion protein. The first fusion protein comprises a first viral protein domain fused to a first peptide fragment of a split reporter. The second fusion protein comprises a second viral protein domain fused to a second peptide fragment of the split reporter. The sensor produces detectable signals only when antibodies against the viral protein domain is present in the patient sample, which brings the first peptide fragment and the second peptide fragment of the split reporter protein within proximity to each other.

The term “fresh,” as in “fresh sensor,” “fresh substrate,” or “fresh buffer,” means that a sensor, substrate, or buffer that has never been lyophilized. A fresh sensor (or substrate or buffer) used in the assays can be one that has been thawed from a frozen stock.

### 3.1 The Viral Protein Domains (V) of the Protein Biosensor

SARS-CoV-2 comprises a positive-strand RNA genome that encodes 16 non-structural proteins, nine accessory factors, and four structural proteins (S, E, M, and N) {Gordon et al., 2020, #79729} as well as accessory proteins with mostly unknown function (Narayanan et al. 2008). Antibodies from COVID-19 patients are predominantly directed against epitopes in the SARS-CoV-2 Spike protein (S Protein) and the SARS-CoV-2 nucleocapsid protein (N Protein). In some embodiments, the viral protein domains of a Protein Biosensor (αV, βV) include sequences from the SARS-CoV-2 spike protein receptor binding domain (“SpikeRBD”). In some embodiments, the viral protein domains of a Protein Biosensor include sequences from the SARS-CoV-2 N protein (“N”). As used herein, the term “SARS-CoV-2 viral protein domain,” refers to a protein domain that include an amino acid sequence from a protein of the SARS-CoV-2 virus, and the amino acid sequence has a length within a range from 100 to 400 amino acids, e.g., from 110 to 300 amino acids, or from 130 to 250 amino acids.

### 3.1.1 Spike Protein Domains

“Spikes” are coronavirus surface proteins that mediate receptor binding and membrane fusion between the virus and host cell. Spikes are homotrimers of the S protein, which has S1 and S2 subunits. The interaction between the SARS-CoV-2 Spike protein and the angiotensin-converting enzyme 2 (ACE2) on human cells is critical for viral entry into host cells (Gralinski & Menachery, 2020; Tai et al., 2020; Wu et al., 2020). The receptor binding domain (RBD) is located on the S1 subunit and can bind to the receptor on target cells.

The S1 subunit includes the receptor binding domain (RBD). See Walls et al., 2020, “Structure, function and antigenicity of the SARS-CoV-2 spike glycoprotein” Cell 181:281 An exemplary SARS-CoV virus-2 Spike RBD protein (or, equivalently, SpikeRBD protein) sequence is the RBD region of the Spike protein, which consists of amino acid residues 328-533 (SEQ ID NO: 1):

RFPNITNLCPFGEVFNATRFASVYAWNRKRISNCVADYSVLYNSAS
FSTFKCYGVSPTKLNDLCFTN VYADSFVIRGDEVRQIAPGQTGKIA
DYNYKLPDDFTGCVIAWNSNNLDSKVGGNYNYLYRLFRKSN LKPFE
RDISTEIYQAGSTPCNGVEGFNCYFPLQSYGFQPTNGVGYQPYRVVVLSF
ELLHAPATVCGP KKSTNL [SEQ ID NO:1]

The receptor binding domain of the Spike protein is poorly conserved between SARS-CoV-2 and other pathogenic human coronaviruses except for SARS-CoV-1 (e.g., MERS-CoV), so the RBD represents a promising antigen for detecting SARS-CoVs specific antibodies in humans (Premkumar et al., Science immunology vol. 5, June 2020. DOI: 10.1126/sciimmunol.abc8413).

The S protein further comprises an N terminal domain (“NTD”) at amino acid residues 16-291 (SEQ ID NO: 43)

It will be understood that the viral protein domain will be bound by antibody(s) from at least some patients specific for the SARS-CoV-2 S protein. That is the viral protein domain should present an epitope that also presented by the viral S protein. In one aspect, the S sensor comprises a viral protein domain that is identical to or substantially identical to SEQ ID NO: 1, 44 or 45.

As used herein, a viral protein domain comprises a sequence is substantially identical to a reference amino acid sequence refers to that the sequence shares substantial amino acid sequence similarity to the reference sequence, or the sequence has substantial activity as the reference sequence, or the sequence shares both substantial amino acid sequence similarity and substantial activity with the reference sequence.

The term “substantial sequence similarity,” refers to the viral protein shares at least 70%, at least 80%, at least 90%, at least 91%, at least 92%, at least 93%, at least 95%, at least 96%, at leaset 97%, at least 98%, or at least 99% sequence identity with the reference amino acid sequence.

The term “substantial activity,” refers to that the binding affinity of a speified reference protein (e.g., N protein or S protein or binding fragment thereof) to a specified binding partner (e.g,. ACE2) is at least 70%, at least 80%, at least 90%, or at least 95% of that of corresponding reference protein to the same binding partner. For example, a SpikeRBD used in the sensor has substantial activity to the wild type SpikeRBD (SEQ ID NO:1) if its affinity to the ACE2 (or ACE2-Fc or an reference antibody that is known to bind the wild type SpikeRBD) is at least 50%, at least 60%, at least 70%, at least 80%, at least 90%, or at least 95% of the affinity of the wild type SpikeRBD to the ACE2 (or ACE2-Fc or the reference antibody).

In one aspect, the viral protein domain comprises a sequence that is identical to, or substantially identical to, any one of SEQ ID NO:1, 43, 44, or 45. In one aspect, the viral protein domain comprises a sequence that is identical to, or substantially identical to, a subsequence of any one of SEQ ID NO:1, 43, 44, or 45 and the subsequence comprises at least 8 contiguous amino acids, at least 10 contiguous amino acids, at least 15 contiguous amino acids, at least 20 contiguous amino acids, at least 25 contiguous amino acids, or at least 50 contiguous amino acids of any one of SEQ ID NO:1, 43, 44, or 45. In some embodiments, the viral protein domain has a sequence that differs from any one of SEQ ID NO: 1, 43, 44, or 45 by no more than one, no more than two, no more than three, no more than four, no more than five, no more than eight, no more than 10 amino acid residues and/or shares a substantial activity of any one of the SEQ ID NO: 1, 43, 44, or 45. One example of a substantial activity of any one of SEQ ID NO: 1, 43, 44, or 45 is that it can be bound by a reference antibody that also binds SEQ ID NO:1, 43, 44, or 45. In some embodiments, the reference antibody for the S sensor can be any known anti-SARS-CoV-2 SpikeRBD antibody, for exampleC004, C105, C135, CR3022, as disclosed in Robbiani et al., 2020 and Yuan et al., 2020, the entire content of which is herein incorporated by reference. The heavy chain and light chain sequences of these exemplary antibodies are known and also provided in the sequence listing. Another example of a substantial activity of SEQ ID NO:1 is that it is capable of binding to the human ACE2 receptor having the sequence of SEQ ID NO: 8 or to a human ACE2 receptor-Fc fusion protein having the sequence of SEQ ID NO: 9.

In some embodiments the antibodies C004, C105, C135, CR3022 compete with patient anti-SARS-CoV-2 antibodies for binding to the viral protein domain.

The binding activity of the SpikeRBD can be assessed by comparing its binding affinity to the human ACE2 receptor-Fc fusion protein (SEQ ID NO: 9) or a reference antibody with the binding affinity of the wild type SpikeRBD to the ACE2 receptor or the reference antibody. Any method that is capable of detecting protein-protein interaction can be used to assess the activity of the SpikeRBD. Non-limiting examples of suitable methods include biolayer interferometry, ELISA and BiaCore.

As described above, the first or the second SpikeRBD may have the same or different sequences. In some approaches, the first peptide fragment is fused to the C-terminus of the first SpikeRBD, and the second peptide fragment is fused to the C-terminus of the second SpikeRBD. Two polypeptide domains of a fusion protein that are “fused” can be directly fused (sequences are contiguous in the fusion protein) or joined by a linker (e.g., a polypeptide linker). In some approaches, the first peptide fragment is fused to the N-terminus of the first SpikeRBD, and the second peptide fragment is fused to the N-terminus of the second SpikeRBD. In some approaches, the first peptide fragment is fused to the C-terminus of the first SpikeRBD, and the second peptide fragment is fused to the N-terminus of the second SpikeRBD. In some approaches, the first peptide fragment is fused to the N-terminus of the first SpikeRBD, and the second peptide fragment is fused to the C-terminus of the second SpikeRBD.

The terms “identical” or percent “identity,” in the context of two or more polypeptide sequences (e.g., the SpikeRBD or the N protein), refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, e.g., the length of the two sequences, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, for purposes of this invention, BLAST 2.0 can be used with the default parameters to determine percent sequence identity.

Sequence identity can be also be determined by inspection. For example, the sequence identity between sequence A and sequence B, aligned using the software above or manually (to maximize alignment), can be determined by dividing the length of sequence A, minus the number of gap residues in sequence A, minus the number of gap residues in sequence B, by the sum of the residue matches between sequence A and sequence B, times one hundred.

### 3.1.2 Nucleocapsid (N) Protein

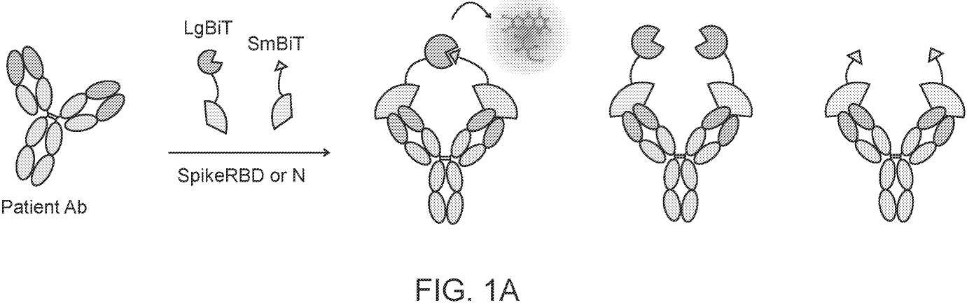

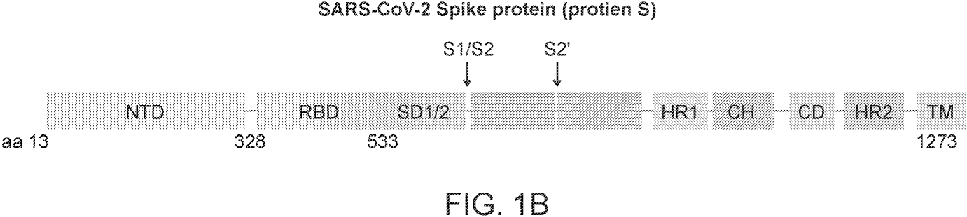

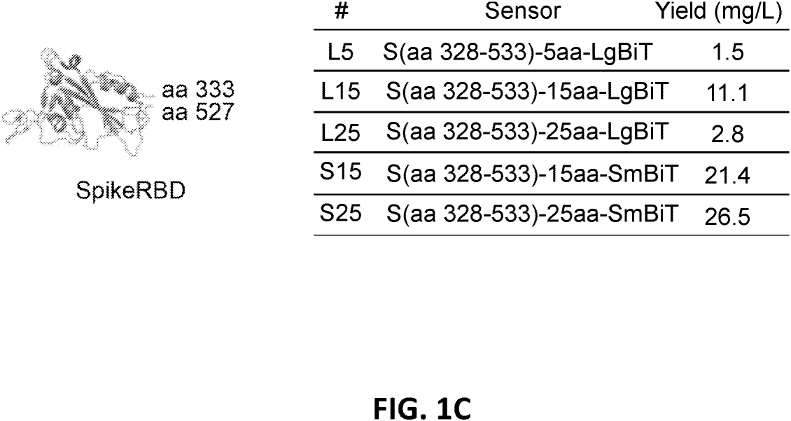

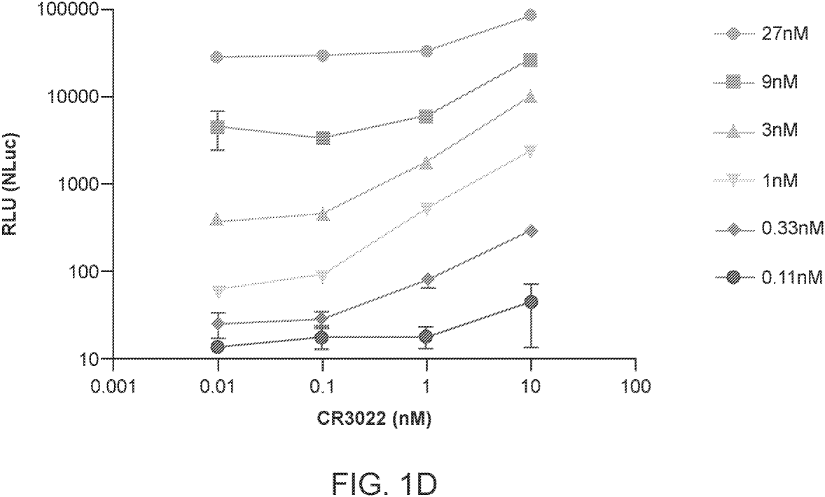

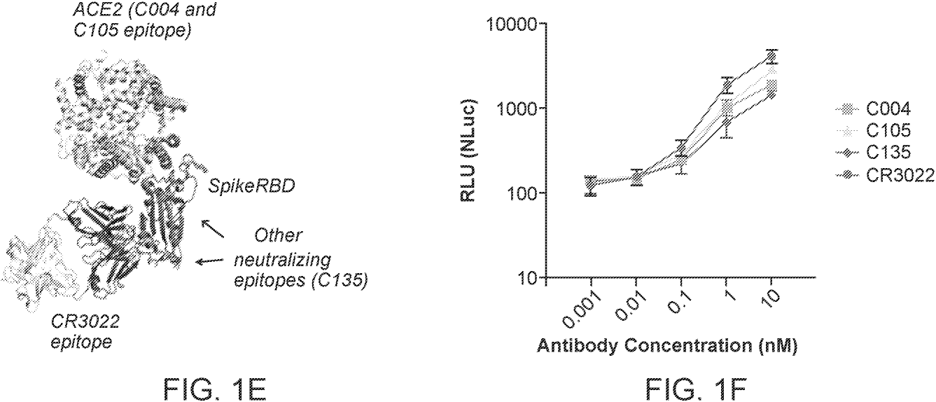

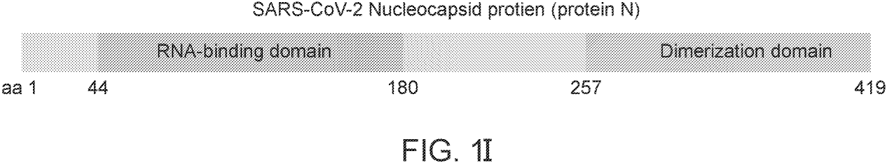

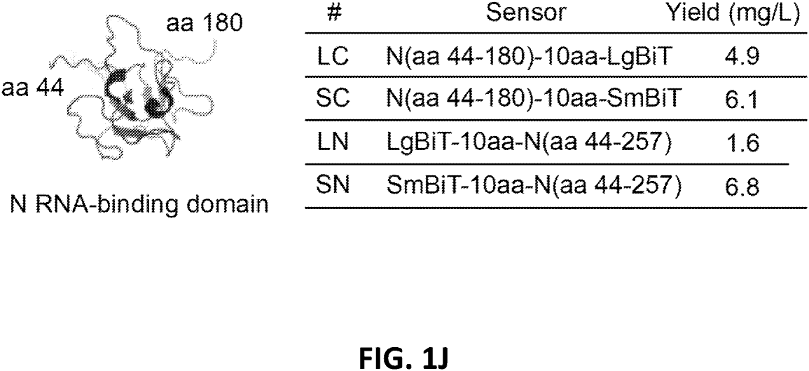

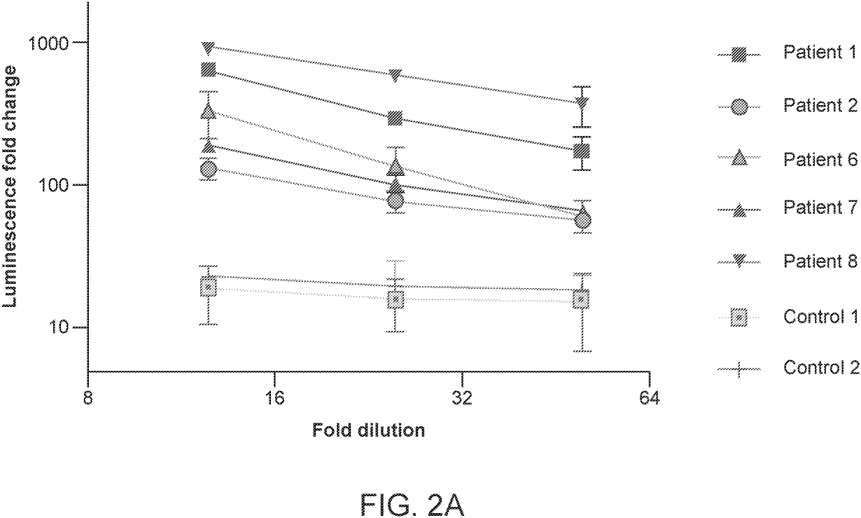

N protein, also called nucleocapsid protein N, packages the viral genome into a ribonucleocapsid and plays a fundamental role during viral self-assembly {Chang et al., 2014, #63528; Zamecnik et al., 2020, #94100}. The N protein comprises an N-terminal RNA binding domain, which aids in viral RNA assembly and packaging into the viral particle. The N protein comprises a C-terminal dimerization domain consists of amino acid residues 258-419 (SEQ ID NO: 7) (FIG. 1I) and an RNA binding domain consisting of aa 44-180. The N-terminal sequences, aa 134-171 (SEQ ID NO: 15), 153-190 (SEQ ID NO: 16), and 210-247 (SEQ ID NO: 17), are immunogenic and have very low similarity with human CoVs other than SAR-CoV-1 and SAR-CoV-2 (5.3%, 2.6%, 0%), but shares 94.7%, 97.4%, 86.8% amino acid sequence identity with SARS-CoV-1. Zamecnik et al., doi.org/10.1101/ 2020.05.11.20092528. Thus, these various N-terminal sequences, including SEQ ID NOs: 5, 6, and 15-17 can be used in the N sensors as described below. An exemplary RNA binding domain of the N protein is SEQ ID NO: 5.

It will be understood that the N protein domain will be bound by antibody(s) from at least some patients specific for the SARS-CoV-2 N protein. That is the N protein domain should present an epitope that also presented by the viral N protein. In one aspect, the N sensor comprises a viral protein domain that is identical to or substantially identical to any one of SEQ ID NO:5, 6, and 15-17. In one aspect, the N protein domain comprises a sequence that is identical to, or substantially identical to, a subsequence of any one of SEQ ID NO:5, 6, and 15-17, and said subsequence comprises at least 8 contiguous amino acids, at least 10 contiguous amino acids, at least 15 contiguous amino acids, at least 20 contiguous amino acids, at least 25 contiguous amino acids, or at least 50 contiguous amino acidsof any one of SEQ ID NO:5, 6, and 15-17.. In some embodiments, the viral protein domain has a sequence that differs from any of the SEQ ID NO:5, 6, and 15-17 by no more than one, no more than two, no more than three, no more than four, no more than five, no more than eight, no more than 10 amino acid residues and/or shares a substantial activity of the respective wild type N protein domains, i.e., one of the SEQ ID NO: 5, 6, and 15-17. One example of a substantial activity of SEQ ID NO:5, 6, and 15-17 is that it can be bound by a reference antibody that also binds SEQ ID NO:5, 6, and 15-17. The reference antibody can be any anti-SARS-CoV-2 antibody that is known as against the N protein domain. Exemplary reference antibodies that bind to the N protein are commercially available, for example, 40588-T62 and 40143-R001, available from Sino biological (Wayne, PA).

The activity of the N protein can be assessed by comparing its binding affinity to a reference antibody with binding affinity of the wild type N protein domain to the reference antibody. The reference antibody can be any known anti-N antibody that can bind to the RNA binding domain of the N protein. Any method that is capable of detecting protein-protein interaction can be used for this the assessment, for example, biolayer interferometry, ELISA or BiaCore. In some approaches, the binding activity of the N protein domain that can be used in the N sensor is at least 70%, at least 80%, at least 90%, or at least 95% of that of the wild type N protein domain, e.g., the RNA binding domain of the N protein, SEQ ID NO:5, 6, and 15-17. The first or the second N protein domain used in the same N sensor may have the same or different sequences.

As described above, the sequences of the first and second N protein domains may be the same or different sequences. In some approaches, the first peptide fragment is fused to the C-terminus of the first N protein domain, and the second peptide fragment is fused to the C- terminus of the second N protein domain, as shown in Example 4, FIG. 1J.

In some embodiments, each fusion protein of the S sensor comprises an S epitope encoded in SEQ ID NO: 1, or a variant sequence of SEQ ID NO: 1 as described above. In some embodiments, each fusion protein of the N sensor expresses an N epitope encoded in sequences of SEQ ID NO: 5, 6, or 15-17. Generally, the term “epitope” refers to the area or region of an antigen (e.g., the S protein or the N protein) to which an antibody specifically binds, i.e., an area or region in physical contact with the antibody. Thus, the term “epitope” refers to that portion of a molecule capable of being recognized by and bound by an antibody at one or more of the antibody’s antigen-binding regions. It is contemplated that the epitope recognized by an anti-SARS-CoV-2 antibody from a patient sample may be a linear epitope or a conformational epitope.

### 3.2 Protein Biosensor Detection Moiety Domains (D)

In one aspect, the invention makes use of split reporters comprising complementary portions. Each of the complementary portions of the split reporter protein is individually inactive; when all the commentary portions bind to one another, they form an active (e.g., enzymatically active) protein complex, which can be detected. Each of the complementary portions of a “split reporter protein” can be referred to as a “polypeptide fragment”, or a “peptide fragment,” e.g., a first peptide fragment and a second peptide fragment).It will be recognized that when the split reporter is a protein, the terms “peptide fragment” and “detection moiety domain” are used interchangeable. The fragments (or the complementary portions) of the split reporter proteins have low affinity for each other and must be brought together by other interacting proteins fused to them. The ability to turn on the split reporter protein activity can be exploited to monitor protein interactions by fusing each peptide fragment of the split protein to different proteins that have affinity for one another. The interaction between these different proteins creates a high local concentration of the peptide fragments, thereby causing the separate fragments of the split protein to bind to one another to form an active protein complex.

### 3.2.1 Split-Luciferase

In some approaches, the split reporter is a split-luciferase. In some embodiments, the luciferase is a split-nanoluciferase. Split-nanoluciferases are commercially available, for example, NanoBiT® from Promega (Madison, WI). The NanoBiT system comprises two subunits: SmallBiT (SmBiT), an 11 amino acid peptide [SEQ ID NO:3], and LargeBit (LgBiT), a 17.6 kDa subunit [SEQ ID NO:4] that binds weakly to SmBiT (Kd = 190 µM). When the SmBiT and LgBiT domains are in close proximity, the two subunits come together to form an active luciferase. See U.S. Pat. No. 9,797,889 B2 and Dixon et al., 2016, “NanoLuc Complementation Reporter Optimized for Accurate Measurement of Protein” ACS Chem. Biol. 11:400-408, both incorporated herein by reference.

In some embodiments, the first fusion protein of the sensor comprises a first peptide fragment having greater than 40% sequence identity with SEQ ID NO: 4 (e.g., >40%, >45%, >50%, >55%, >60%, >65%, >70%, >75%, >80%, >85%, >90%, >95%, >98%, >99%), and/or the second fusion protein of the sensor comprises a second peptide fragment comprising SEQ ID NO: 3, and a detectable bioluminescent signal is produced or substantially increased (e.g., increase at least 1.5 fold, at least 2 fold, at least 5 fold, at least 10 fold, or at least 15 fold) when the first peptide fragment contacts the second peptide fragment. In some embodiments, the second peptide fragment has a sequence having one, two, or three single amino acid mutations (substitutions, deletions, or insertions) relative to SEQ ID NO: 3.

In one illustrative example, the protein biosensor detection moiety domains are split fragments of a luciferase. In one illustrative example, the protein biosensor detection moiety domains, SmBiT and LgBiT, split fragments of a Nanoluciferase (NanoLuc®) as anti-SARS-CoV-2 antibody sensors (FIG. 1A).

The mechanism of the antibody biosensors is dependent on the multimeric nature of a human immunoglobin (Ig) molecule. When incubating the viral antigen/LgBiT or SmBiT fusions with patient serum, a population of the antibody Ig molecules will have one of its two Fragment Antigen Binding (Fab) arms binding the LgBiT fusion, and the other binding the SmBiT fusion (FIG. 1A). This hetero-bivalent interaction will localize the LgBiT and SmBiT fragments in close proximity, resulting in the reconstitution of an intact, active NanoLuc enzyme (FIG. 1A).

Other luciferase-based split reporter systems may be used in the present invention. See, Cassonnet et al., 2011, “Benchmarking a luciferase complementation assay for detecting protein complexes” Nature Methods. 8 (12): 990-2), including ReBiL (Li et al. (2014). “A versatile platform to analyze low-affinity and transient protein-protein interactions in living cells in real time” Cell Reports 9 (5): 1946-58), gaussia princeps luciferase (Neveu et al. (2012). “Comparative analysis of virus-host interactomes with a mammalian high-throughput protein complementation assay based on Gaussia princeps luciferase”. Methods. 58 (4): 349-59), and NanoBiT® (Promega).

### 3.2.2 Other Split Reporter Systems

Additional reporter proteins include horseradish peroxidase or HRP (Martell at al. (2016). “A split horseradish peroxidase for the detection of intercellular protein-protein interactions and sensitive visualization of synapses”. Nature Biotechnology. 34 (7): 774-80), engineered soybean ascorbate peroxidase (APEX2); β-lactamase (Park et al. (2007). “Bacterial beta-lactamase fragmentation complementation strategy can be used as a method for identifying interacting protein pairs,” Journal of Microbiology and Biotechnology. 17 (10): 1607-15), β-galactosidase (Rossi et al. (1997). “Monitoring protein-protein interactions in intact eukaryotic cells by beta-galactosidase complementation”. Proceedings of the National Academy of Sciences of the United States of America. 94 (16): 8405-10), dihydrofolate reductase (Tarassov et al. (2008). “An in vivo map of the yeast protein interactome” (PDF). Science. 320 (5882): 1465-70), Green Fluorescent Protein (GFP) and GFP variants (Barnard et al. (2010). “Split-EGFP Screens for the Detection and Localisation of Protein-Protein Interactions in Living Yeast Cells”. Split-EGFP screens for the detection and localisation of protein-protein interactions in living yeast cells. Methods in Molecular Biology. 638. pp. 303-17; Blakeley et al. (2012). “Split-superpositive GFP reassembly is a fast, efficient, and robust method for detecting protein-protein interactions in vivo”. Molecular BioSystems. 8 (8): 2036-40; Cabantous et al. (2013). “A new protein-protein interaction sensor based on tripartite split-GFP association”. Scientific Reports. 3: 2854; MacDonald et al. (2006). Nat Chem Biol 2006, 2, 329-337; Hu et al. (2003). Nat Biotechnol 2003, 27, 539-45), ubiquitin Duenkler et al. (2012). “Detecting Protein-Protein Interactions with the Split-Ubiquitin Sensor”. Detecting protein-protein interactions with the Split-Ubiquitin sensor. Methods in Molecular Biology. 786. pp. 115-30), Tobacco Etch Virus (TEV) protease (Wehr et al. (2006). “Monitoring regulated protein-protein interactions using split TEV”. Nature Methods. 3 (12): 985-93), focal adhesion kinase (Ma et al. (2014). “A new protein-protein interaction sensor based on tripartite split-GFP association”. Scientific Reports. 3: 2854), and infrared fluorescent protein IFP1.4 (Tchekanda et al. (2014), “An infrared reporter to detect spatiotemporal dynamics of protein-protein interactions”. Nature Methods. 11 (6): 641-4); Michnick et al., Nat Rev Drug Discov 6, 569-82 (2007); Remy & Michnick, Methods Mol Biol 1278, 467-81 (2015); Morrell et al., FEBS Lett 583, 1684-91 (2009)), split protein complementation (Shekhawat & Ghosh, Curr Opin Chem Biol 15, 789-97 (2011)), or bimolecular fluorescence complementation (Miller et al., 2015, J Mol Biol 427, 2039-55; Kerppola, T. K., 2009, Chem Soc Rev 38, 2876-2886). Also see SS Shekhawat, 2011, “Split-protein systems: beyond binary protein-protein interactions”Current opinion in chemical biology - Elsevier

### 3.2.3 Nonprotein Detection Moiety Domains

The reporter moieties (also referred to as detection moiety in this disclosure) of the split reporter can also be nucleic acids or other moieties that associate when bound in proximity to each other (optionally in the presence of accessory reagents).

In some approaches, proximity extension assay and the proximity ligation assays are used to detect antibodies against SARS-CoV-2. In Proximity Extension Assays and Proximity Ligation Assays probes (a pair of nucleic acid moieties, and each attached to a viral protein domain) are brought into proximity in the presence of the antibodies against the viral protein domains. If the two probes bind close together (e.g., bind the Spike protein, or bind two different Spike proteins on the same virion) the nucleic acid moieties interact by hybridization to each other, or hybridization to a common splint oligonucleotide, to form a complex. The complex can then be detected by ligation, extension and/or amplification of the nucleic acid complex. See, US Pat. No. 6,878,515; US Pat. No. 7,306,904; Fredriksson et al., 2002, “Protein detection using proximity-dependent DNA ligation assays.” Nat. Biotechnol. 20:473-477; Lundberg et al., 2011, “Homogeneous antibody-based proximity extension assays provide sensitive and specific detection of low-abundant proteins in human blood” Nucleic Acids Res. 39:e102. In these approaches, the first viral protein domain is linked to the first nucleic acid probe and the second viral protein domain is linked to the second nuclear acid probe in a system that is adapted for a proximity ligation assay, proximity extension assay or other nucleic acid based proximity assay (e.g., the two polynucleotides are partially complementary to each other or are both partially complementary to an oligonucleotide in the mixture).

It will be understood that, in the case the Proximity Extension or Proximity Ligation Assays are used, the detection moiety domains of fusion proteins are replaced by polynucleotide domains.

### 3.2.4 Detection

The association of the first reporter moiety and the second reporter moiety can be detected based on enzymatic activity, probe amplification, or other split reporter methodologies are well known and have been used for the detection and/or quantification of protein interactions. As noted above, the reconstitution of the reporter protein (by the association of the first and second peptide fragments of the split reporter protein) produces an enzymatically active reporter that, in presence of suitable substrates and/or accessory reagents generates a detectable signal. Detectable signals include, without limitation, colorimetric, fluorescent and luminescent signals. In some embodiments, the substrate for the split reporter protein is luciferin, furimazine, or some other luminogenic substrate or molecule. In one embodiment, the reporter is a luciferase, and a substrate for the luciferase (e.g., coelenterazine, furimazine, luciferin, or some other luminogenic substrate) is added to the reaction mixture. After the addition of the substrate, the reaction mixture may be incubated for a sufficient amount of time to allow the development of the signal. The step of signal development may last between 5 to 30 minutes, for example, about 10 minutes.

As disclosed herein, in some embodiments, a cutoff can be determined from a reference population (consisting of e.g., at least 2, at least 3, at least 5, at least 10, at least 20, or at least 50 healthy individuals) and can be used determine if the patient has developed antibodies that can bind the protein sensor used in the assay. In some embodiments, the cutoff is the mean or median of signals from control samples that are known to be negative for the antibodies (e.g., samples from healthy individuals). In some embodiments, a cutoff can be determined as 1 fold, 1.2 fold, 1.5 fold, 2 fold, 3 fold, 4 fold, 5 fold of the mean or median of signals from the control samples. In some embodiments, the cutoff is a the mean (or median) plus three standard devations of signals of the reference population consisting of healthy individuals.

### 3.2.4.1 Luminometer

The luminescent signals can be read by a luminescence microplate reader (e.g. Tecan Infinite 200 Pro, Promega GloMax), a portable luminometer (Junior LB9509), a hand-held ATP luminometer with customized sample tube (3M™ Clean-Trace™ Hygiene Monitoring and Management System), or a home-made luminometer to improve detection sensitivity and decrease required sample volume. The luminescent signals can also be read using an app on a mobile phone or with an adaptor to a mobile phone camera.

The split luciferase assay disclosed herein is amenable for high-throughput runs with automation platforms. For example, a simulated run for 40 plates (3,840 assays) can be completed in 3 h on an automation workflow using the University of California, San Francisco (UCSF) Antibiome Center robotics platform. Serum sample transfer to an assay plate using Biomek Fx Automated Workstation may be completed in about 2 minutes. Robotics-assisted dispensing and luminescence reading for one iteration of 96 assays is expected to take about 35 minutes.

### 3.3. Protein Biosensor Linker Domains

In some approaches, a viral protein domain is fused to one member of the complementary portions of the split reporter via a linker. In some approaches, one member of the two complementary portions of the split reporter is fused to a first viral protein domain via a first linker, and the second viral protein domain is fused to the other member of the two complementary portions of the split reporter via a second linker.

In some approaches, the linker is a polypeptide linker. A linker used for the sensors may contain synthetic or natural sequences. In preferred embodiments, the linker used in the invention described herein is a flexible linker. As used herein, the term “flexible linker” refers to a linker configured to allow protein domains joined by the linker to have a certain degree of movement or interaction. Flexible linkers are generally rich in small or polar amino acids such as gly and Ser to provide good flexibility and solubility. In some embodiments, the flexible linker is a GS linker, i.e., linkers having sequences consisting primarily of stretches of Gly and Ser residues, e.g., at least 90%, or at least 95%, or at least 98% or at least 99%, or all of the amino acid residues of linker are either Gly or Ser. In some cases, the GS linker is one or more repeats of GGGGS (SEQ ID NO:18), or GSSGSS (SEQ ID NO:20). Other types of flexible linkers include, but are not limited to, GGGGGGGG (SEQ ID NO: 21) and GSAGSAAGSGEF (SEQ ID NO:22), GGGGSGGGGSGGGGS (SEQ ID NO: 23), GGGGSGGGGSGGGGSGGGGSGGGGS (SEQ ID NO: 19). Additional non-limiting exemplary linkers that can be used include those disclosed in Chen et al., Adv. Drug. Deliv. Rev. 65 (10): 1357-1369 (2014) and Rosemalen et al., Biochemistry 2017, 56, 50, 6565-6574, the entire contents of both publications are herein incorporated by reference.

The first linker and the second linker may have the same or different amino acid sequence. The first linker and the second linker may also be of the same or different length.

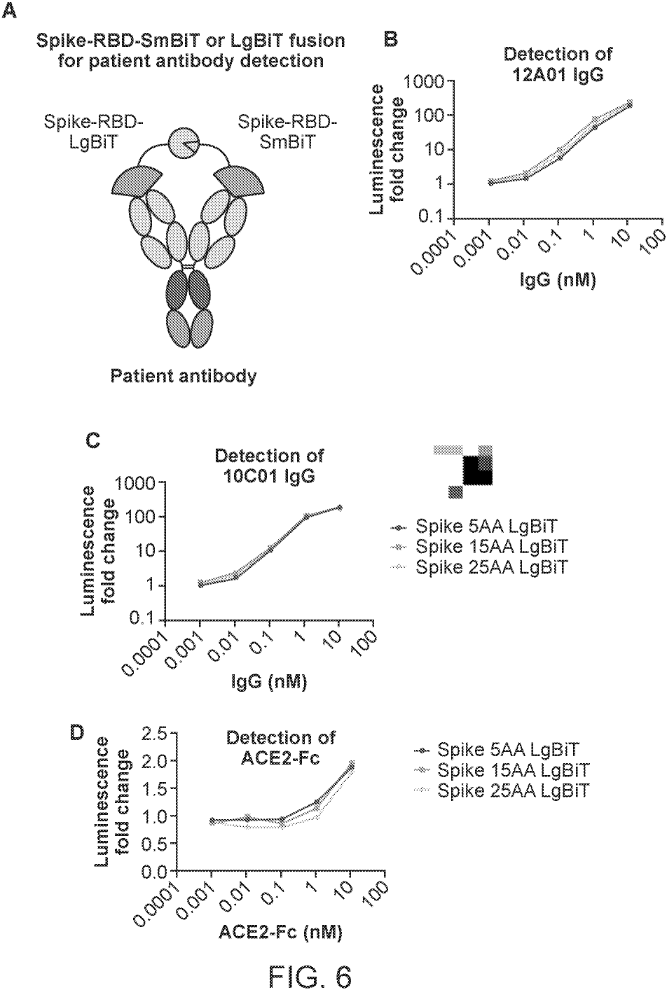

The length of the linkers used in the sensors may vary; in some cases, the linker may have a length that ranges from 1- 50 amino acids, e.g., 1 to 25, 10 to 25, or 15 to 40 amino acids. The linker’s length may affect the yield of the viral protein domain-Linker-detection moiety (VLD) fusion protein. In one illustrative example, a fusion protein having a protein linker having a length of 15 amino acids, which links the SpikeRBD and the LgBiT, resulted in the highest protein yield as compared to linkers having a length of either 25 amino acids or 5 amino acids. In another illustrative example, using a protein linker having a length of 25 amino acids to link SpikeRBD and SmBiT produced a fusion protein with higher protein yield than does a protein linker of 15 amino acids. See FIG. 1C and Example 1. In another illustrative example, using the linker having a length of 10 amino acids to link an N protein domain with the LgBiT or the SmBiT domains showed moderate protein yield that is acceptable to be used as N sensor in the solution serological assay. See FIG. 1J, and Example 4. The linker’s length may also affect the sensitivity of the detection. In one illustrative example (FIG. 5A), a fusion protein having a protein linker having a length of 15 or 25 amino acids, which links the SpikeRBD and the LgBiT, resulted in the higher detection sensitivity as compared to linkers having a length of 5 amino acids.

Optionally, the polypeptide linker may have some function in addition to joining the viral protein domain and the detection moiety in-frame. For example, the linker may comprise additional sequences (e.g., sequence tags) that can facilitate purification of the fusion protein.

### 4. Exemplary Protein Biosensors

### 4.1 Orientation

The biosensors used in the invention may take different forms. A biosensor typically includes two fusion proteins, each comprising a split reporter fragment and a viral protein domain. In some embodiments, the split reporter fragment is be fused at the end (i.e., the C terminus or the N terminus) of a viral protein domain (e.g., the Spike RBD or the N protein) to use as protein sensors. In some embodiments, the split reporter fragment is internal (flanked by other portions of the fusion polypeptide as protein sensors. In some embodiments, the biosensor comprises two fusion proteins, in which each of the split reporter fragments is fused to the N terminus of a viral protein domain. In some embodiments, the biosensor (e.g., an N sensor) comprises two fusion proteins, in which each of the split reporter fragments is fused to the C terminus of a viral protein domain. In some embodiments, the biosensor comprises two fusion proteins; in one fusion protein, the split reporter fragments is fused to the C terminus of a viral protein domain, and in the other fusion protein, the split reporter fragments is fused to the N terminus of a viral protein domain. An exemplary N sensor of such configuration is an N (aa 44-180)-LgBiT (SEQ ID NO:10) and N (aa 44-180)-SmBiT (SEQ ID NO:11), as shown in Example 4.

In some cases, the two viral domains in the sensor are the same, i.e., having the same sequence. In some cases, the two viral domains in the sensor are different, i.e., having different sequences. The protein sensor is capable of detecting the presence of antibodies as long as the antibodies can recognize both the first and the second viral protein domains.

### 4.2 Additional Sequences

In some embodiments, the one or both of fusion proteins in the biosensor may comprise additional sequences. These additional sequences may have other properties, for example, those that can facilitate expression, purification, folding, or detection of the fusion proteins.

### 4.3. The Spike Protein Sensor (S Sensor)

In some embodiments, the first SpikeRBD is fused to a LgBiT domain via a first linker, and the second SpikeRBD is fused to a SmBiT domain via a second linker. The first linker and the second linker may have a length that ranges from 1- 50 amino acids, e.g., 1 to 25, 10 to 25, or 15 to 40 amino acids. S sensors comprising SpikeRBD are also referred to as the SpikeRBD sensors.

In some embodiments, an S sensor of the invention contains a first linker that has a length of 15 amino acids and a second linker has a length of 25 amino acids. See Example 1 and FIG. 1C, which shows the S sensor having this configuration advantageously can be produced in high yield.

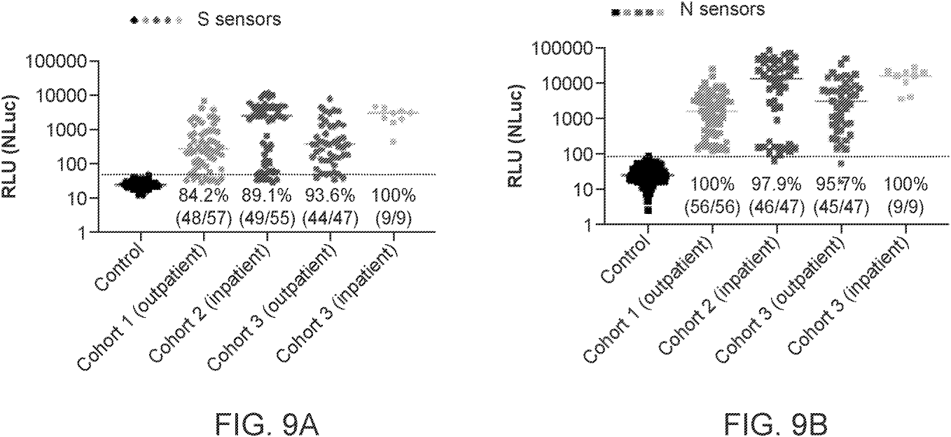

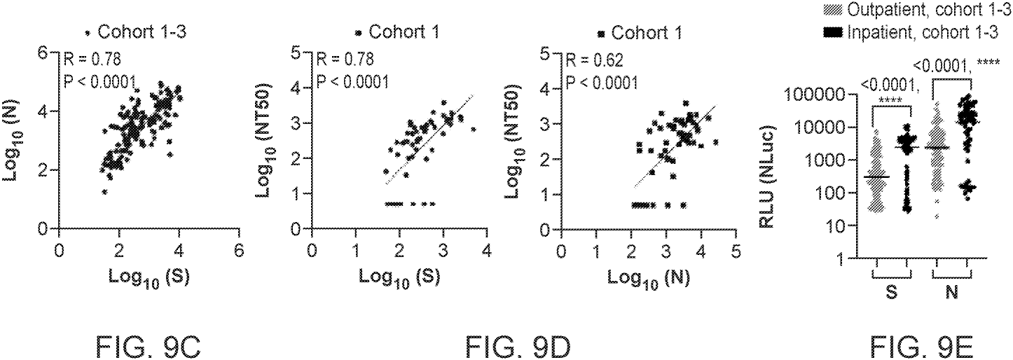

The first fusion protein comprises a first SpikeRBD fused to a first peptide fragment of the split reporter, and a second fusion protein that comprises a second SpikeRBD fused to a second peptide fragment of the split reporter, and the first and second peptide fragments are complementary portions of the split reporter. In some approaches, the first or the second SpikeRBD has a sequence that shares substantial sequence similarity with SEQ ID NO: 1 and also retains the substantial activity of the wild type SpikeRBD (SEQ ID NO: 1). In one exemplary embodiment, the S sensor comprises a first fusion protein comprising a SpikeRBD domain having a sequence of SEQ ID NO:1 fused to a split reporter fragment having the sequence of SEQ ID NO: 4, and a second fusion protein comprising a second SpikeRBD having a sequence of SEQ ID NO:1 fused to a split reporter fragment having the sequence of SEQ ID NO: 3. In one example, the S sensor comprises a first fusion protein of S(aa 328-533)-15aa-LgBiT (SEQ ID NO: 12) and a second fusion protein of S(aa328-533)-25aa-SmBiT (SEQ ID NO: 13). This S sensor is referred to as the “L15 + S25” sensor in this disclosure. As shown in FIG. 8A, the L15 +S25 sensors generated linear, dose dependent signals with commercial anti-S protein antibody. In one example, the S sensor comprises a first fusion protein of S(aa 16-291)-15aa-LgBiT (SEQ ID NO: 42) and a second fusion protein of S (aa 16-291)-25aa-SmBiT (SEQ ID NO: 41). S sensors comprising the NTD (aa 16-291) of the Spike proteins fused to the split fragments of the reporter protein are also referred to as the SpikeNTD sensors or NTD sensors. FIG. 13 shows that the NTD sensor was able to detect anti-Spike-NTD antibodies in convalescent patient serum samples.

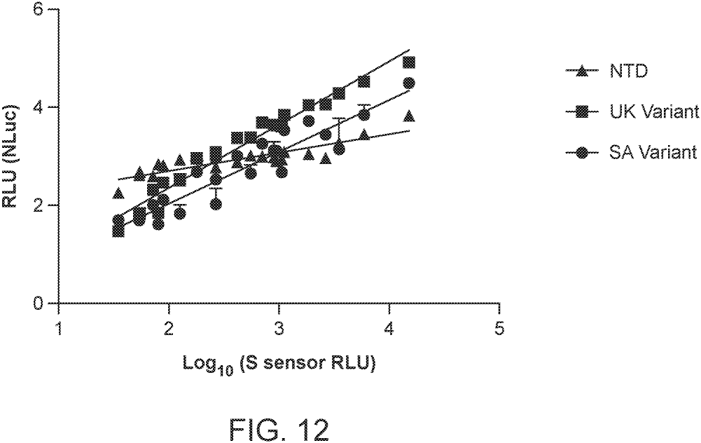

S sensors and N sensors can also be constructed from viral proteins of COVID-19 variants, e.g., the UK variant and the SA variant using the methods disclosed herein. The UK variant (B.1.1.7) comprises an N501Y mutation in the RBD and the SA variant (B.1.351) comprises K417N, E484K, N501Y mutations in the RBD. See, www.cdc.gov /coronavirus/2019-ncov/science/science-briefs/scientific-brief-emerging-variants.html. The SpikeRBD of each variant can be fused to the LgBiT or the SmBiT to contruct the S sensors. In some embodiments, the S sensor comprises a first fusion protein of SEQ ID NO: 37 and a second fusion protein of SEQ ID NO: 38. In some embodiments, the S sensor comprises a first fusion protein of SEQ ID NO: 39 and a second fusion protein of SEQ ID NO: 40. As shown in FIG. 12, these S sensors were able to detect the presence of the anti Spike antibody CR3022 and also in patients recovered from infections of these COVID-19 variants.

### 4.4 The N Protein Sensor

In one aspect the protein sensor is an N protein biosensor (also called the “N Sensor”). In one embodiment, the N sensor comprises a first fusion protein that comprises a first N protein domain fused to a first peptide fragment of the split reporter and a second fusion protein that comprises a second N protein domain fused to a second peptide fragment of the split reporter. In some approaches, the first or the second N protein domain has a sequence that is substantially identical to any of SEQ ID NO: 5, 6, and 15-17. In some approaches and the first and the second N protein domains each have a sequence that is substantially identical to SEQ ID NO: 5, 6, and 15-17 and also retains the substantial activity of the corresponding wild type N-terminal domains of the N protein (SEQ ID NO: 5, 6, and 15-17). In some approaches, the N protein domain used in the N sensor does not include the C-terminal dimerization domain (SEQ ID NO: 7) to avoid formation of N protein dimers. For purposes of this disclosure, N protein domains that exclude the C-terminal dimerization domain are referred to as the N-terminal domains.

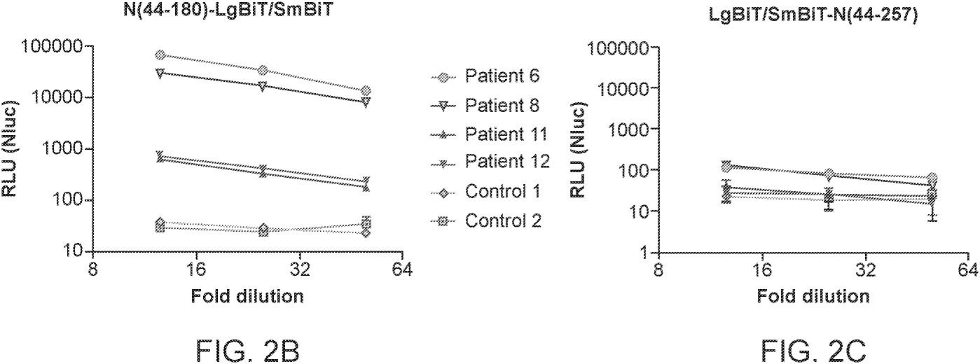

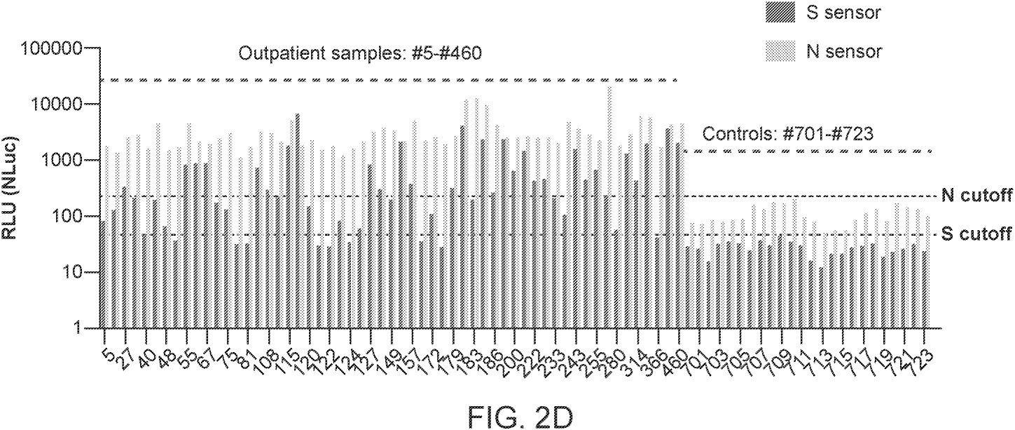

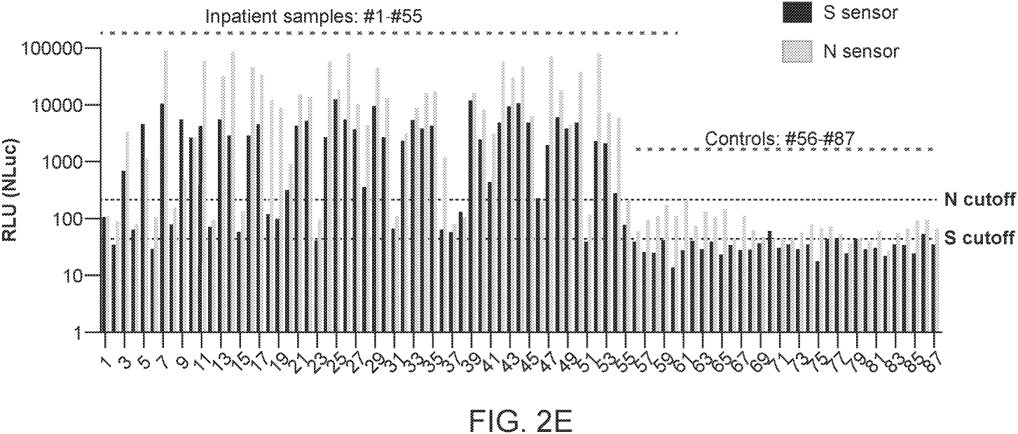

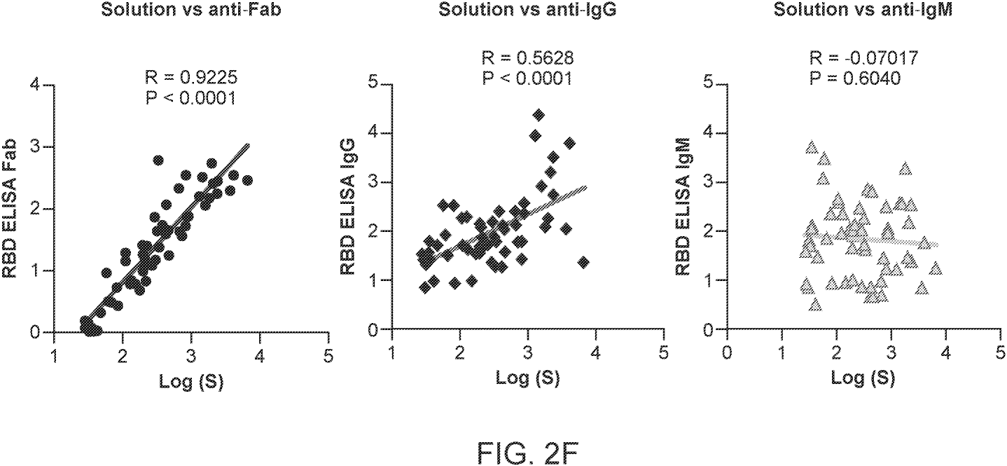

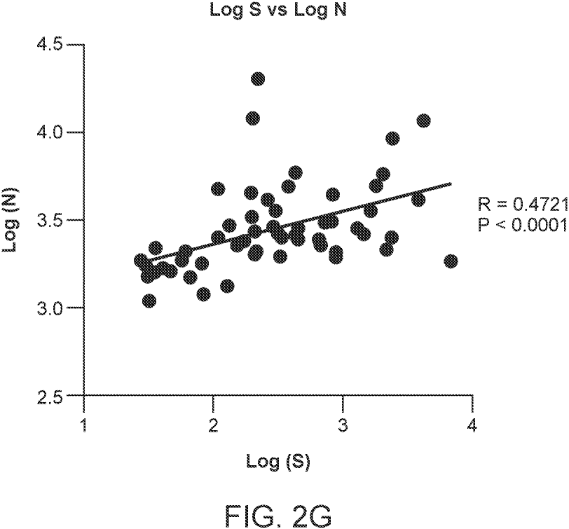

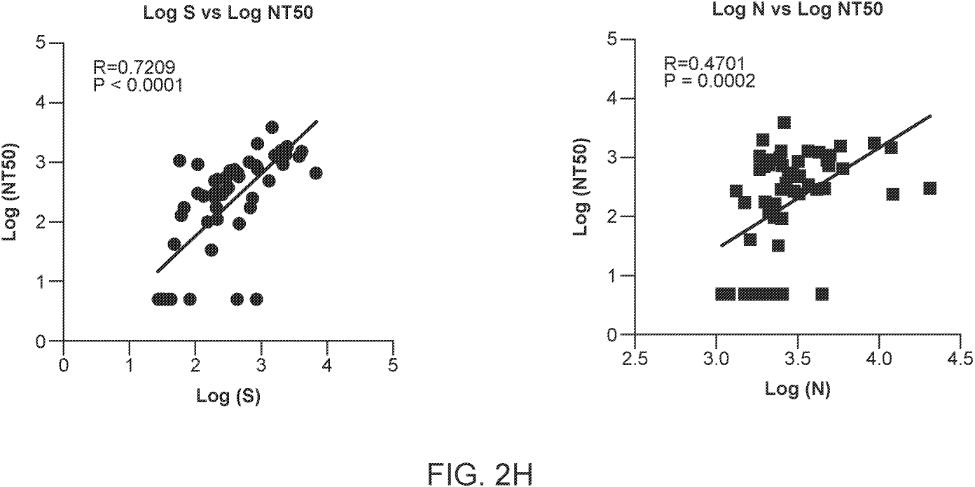

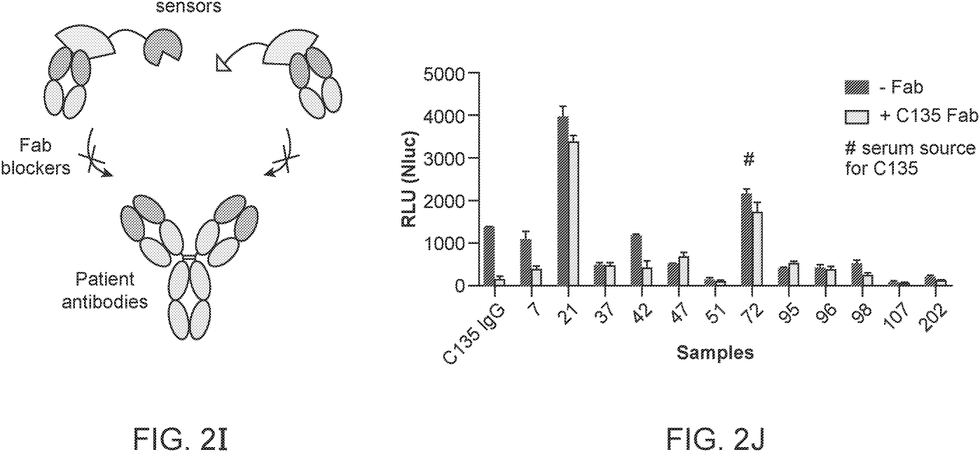

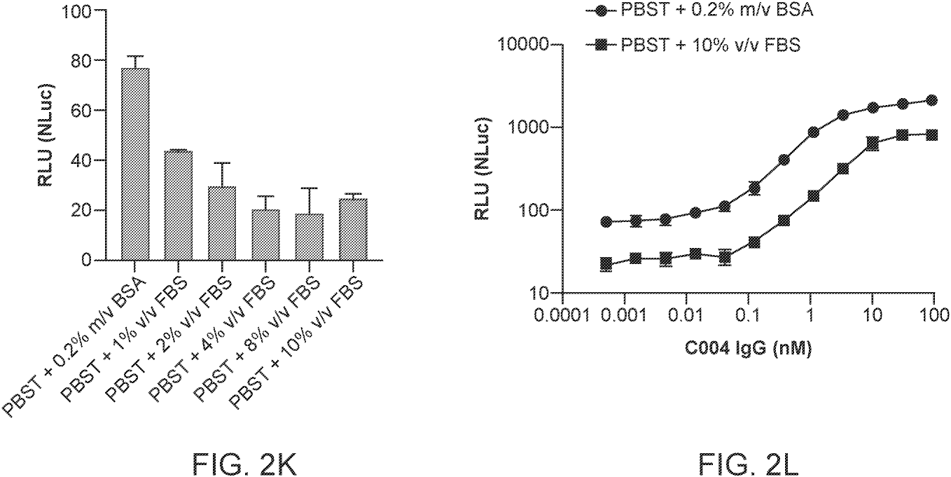

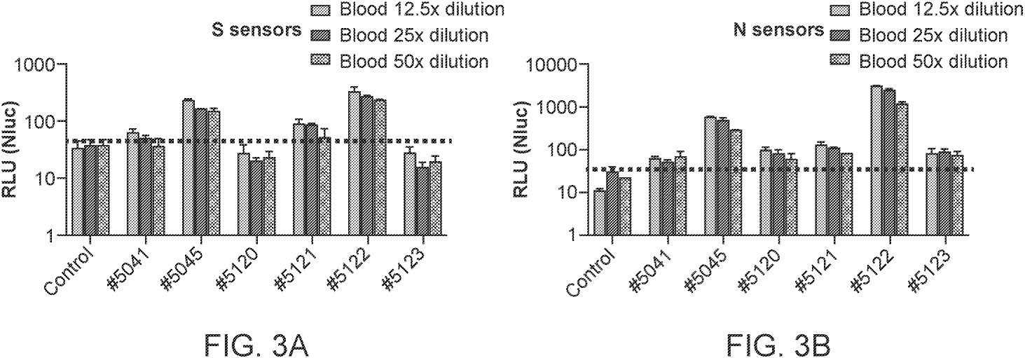

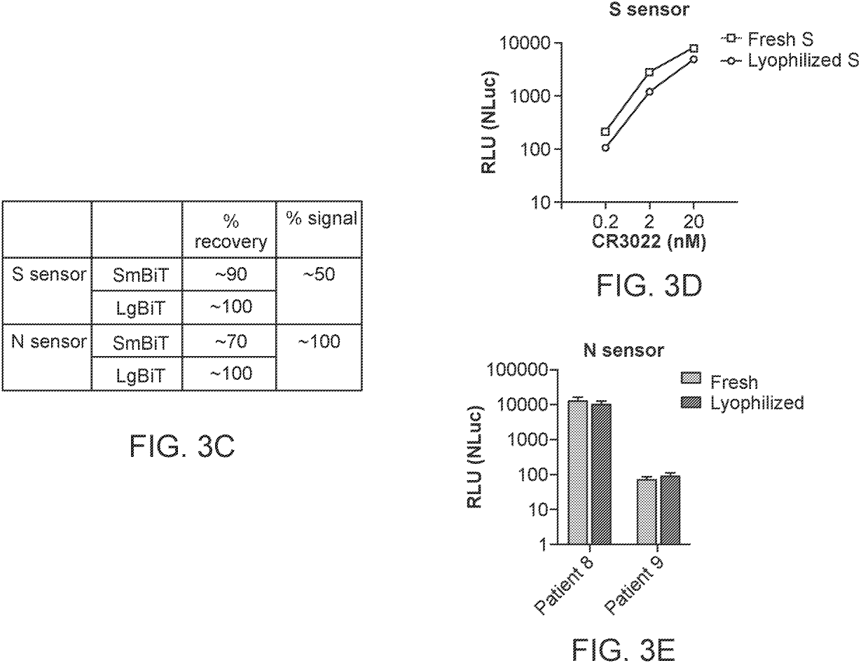

The position of the peptide fragment of the split reporter relative to the N protein domain may affect the assay sensitivity. It is desirable that each peptide fragment is fused to the C-terminus of the N protein domain. One illustrative example of the fusion proteins of the N sensor is shown in Example 4, FIG. 1J. In Examples of this disclosure, “LC” and “SC” represent the C-terminal fusions, in which the LgBiT and the SmBiT are fused to the C terminus of a viral protein domain (the SpikeRBD or the N protein domain), respectively. “LN” and “SN,” are the N-terminal fusions, in which the LgBiT and the SmBiT are fused to the N terminus of a viral protein domain, respectively. In some embodiments, the N sensors used in the methods and compositions are the C-terminal fusions of the N sensors. The LC and SC sensors (N(aa 44-180)-LgBiT/SmBiT) detected patient Abs from all four patients tested (FIG. 2B), while the LN and SN sensors (LgBiT/SmBiT-N(aa 44-257)) only detected Abs from two patient sera samples that had the strongest seropositivity (FIG. 2C). In addition, LC and SC sensors produced much stronger signals than the LN and SN sensors. This indicates that the C-terminal fusions of the N sensors are more sensitive than the N-terminal fusions of the N sensors.

The two N protein domains may be fused to respective split reporter fragments via a linker, and the linker may have a length that ranges from 1- 50 amino acids, e.g., from 1 to 25, from 10 to 25, from 15 to 40 from 5 to 20 amino acids, or about 10 amino acids. In one illustrative example, an N sensor is constructed by fusing a LgBiT to the C terminus of the RNA binding domain of the N protein (SEQ ID NO: 5) via a first linker and fusing a SmBiT to the same RNA binding domain of the N protein via a second linker. Both linkers have an equal length of 10 amino acids. The sensor showed moderate but sufficient yield when expressed in vitro (Example 4 and FIG. 1J); and was able to detect the antibodies from all four (4) patients tested (Example 5, FIGS. 2B and 2C). In some exemplary embodiments, an N sensor comprises a first fusion protein N (aa 44-180)-LgBiT (SEQ ID NO: 10) and a second fusion protein having a sequence of N (aa 44-180)-SmBiT (SEQ ID NO: 11). In some embodiments, the N sensor comprises a first fusion protein LgBiT-N(aa 44-257) and a second fusion protein SmBiT-N(aa 44-257) (also referred to as the N terminal fusion “LN+SN”). In some embodiments, the N sensor comprises a first fusion protein N(aa 44-257)-LgBiT and a second fusion protein N(aa 44-257)-SmBiT (also referred to as C-terminal fusion “LC+SC”). In some embodiments, the N sensor comprises a first fusion protein N(aa 44-257)-LgBiT (SEQ ID NO:36) and a second fusion protein N(aa 44-257)-SmBiT (SEQ ID NO:36) (also referred to as C-terminal fusion “LC2+SC2”). As shown in Table 7 and Table 8, the C terminal fusions, i.e., LC + SC and LC2 + SC2 sensors generated stronger signals over LN + SN (Table 8). In addition, the LC + SC sensors generated linear, dose dependent signals with commercial anti-N protein antibody (FIG. 8B). As shown Table 9, the 6 out of 8 patients showed signal above controls in the serological assay performed with LN + SN sensors, while all four patients showed signals with the LC + SC sensors.

### 5. Serological Assays and Saliva Assays

The present invention includes methods and systems for the detection of antibodies against the Spike protein or the N protein of the SARS-CoV-2. Suitable methods typically include: receiving or obtaining (e.g., from a patient) a sample of bodily fluid or tissue likely to contain antibodies; contacting (e.g., incubating or reacting) a sample to be assayed with a viral protein biosensor disclosed herein, under conditions in which, only if the test sample comprises antibodies that bind the first and second viral protein domains, the first peptide fragment and the second peptide fragment associate to produce a detectable reporter protein; and detecting the association of the first peptide fragment and the second peptide fragment if the patient sample comprises antibodies against the SARS-CoV-2 viral protein.

### 5.1 Sample Dilution

In some approaches, the sample is first diluted in a buffer before testing to minimize the interference from other components in the sample. In some cases, serial dilutions of the sample are made to ensure at least some dilutions are within the dynamic range of the assay and ensure accuracy. The dilution factor can be 1:1 to 1:200, for example, between 1:2 and 1:100, between 1:2 and 1:50, between 1:2 and 1:40, between 1:5 and 1:35, or between 1:10 and 1:30, end points inclusive. In some illustrative examples, the serum samples from the patients are diluted 1:12.5 for the S senor test and 1:25 for the N sensor test. In some illustrative examples, the serum samples from the patients are diluted 1:12.5 for the S senor test and 1:12.5 for the N sensor test.

### 5.2 Sensor Concentration

In some embodiments, the first fusion protein and the second fusion protein of the sensor are present in the reaction mixture at about equal molar concentration to maximize the formation of the active reporter when the target antibodies are present. The term “about equal molar concentration,” refers to a difference between the molar concentrations of the two molecules less than 30%, less than 20%, no greater than 10%, less than 5%, or less than 3% of the lesser value of the two molar concentrations. It is also desirable to maintain the sensor concentration in the reaction mixture within an optimal range to obtain sufficiently high antibody-specific signal while minimizing background readings. In one illustrative example, using 1 nM S sensor produced a wider dynamic range - the assay was capable of detecting ACE-Fc in a range at least from 0.01 nM to 10 nM- compared to assays using the S sensor at higher or lower concentrations, i.e., 0.11 nM, 9 nM, and 27 nM. 0.33 nM or 3 nM sensors produced slightly worse dynamic range. See, Example 1 and FIG. 1D. In another illustrative example, the N sensor was used at a concentration of 1 nM, which was able to detect the anti-N protein antibodies in patient samples in a dose-dependent manner. See Example 5 and FIGS. 2B and 2C. Thus, in some aspects, a protein sensor used in the assay, that is, each of the first and second fusion proteins of the protein sensor, is present in a concentration that ranges from 0.1 nM to 10 nM, e.g., from 0.2 nM to 10 nM., from 0.3 nM to 3 nM, from 0.5 nM to 2 nM, or about 1 nM.

The patient sample and the sensors can be incubated under conditions suitable for the specific binding of the viral protein domains to the antibodies that are against the viral protein domain. The reaction and incubation are typically performed at ambient temperature, i.e., a temperature that is within the ranges of from 10° C. to 40° C., e.g., from 15° C. to 30° C., or from 18° C. to 25° C.

This assay is very easy to perform in a laboratory or a point-of-care location equipped with basic liquid handling devices and a luminescence plate reader or hand-held luminometer. It can be performed in a small reaction volume (<50 µl) in a 384-well plate and only requires 1 nM of each recombinant sensor. Furthermore, as discussed in Section 5.5, the assay format can be adapted to incorporate competitive Fab binders to reveal SpikeRBD epitopes targeted by patient antibodies, which provides important insights for the development of antibody therapeutics using convalescent patient serum.

### 5.3 Exemplary Assay Conditions

Typically, the binding reaction between the antibodies in the patient sample and the sensors is in a solution that has a substantially neutral pH. A substantially neutral pH refers to a pH within a range from 6 to 8, for example, from 6.5 to 7.5, or about 7. A variety of buffers that have substantially neutral pH and that are suitable for antigen-antibody binding can be used for methods disclosed herein, including buffers that are typically used for ELISA, e.g., PBS, TBS. In some approaches, the reaction mixture comprises Bovine Serum Albumin (BSA) and/or Fetal Bovine Serum (FBS), which are present in suitable amounts to minimize non-specific binding and reduce the background of the test (FIG. 2K).

The patient sample may be incubated with the viral protein sensor under the room temperature for a period that is sufficient to allow antibodies to bind to the viral protein domains, which will bring the two fusion proteins into proximity to form the active reporter protein, e.g., a luciferase. In some approaches the length of incubation time ranges from 5 minutes to 1 hour, e.g., from 10 minutes to 30 minutes, or about 20 minutes. In an exemplary embodiment, the N sensor and S sensor were incubated with the test sample at room temperature for 20 minutes, producing good signal indicative of the presence of the antibodies that recognize the Spike protein or the N protein. See FIGS. 2B and 2C.

The methods as disclosed herein significantly reduces the time required for detection of SARS-CoV-2 antibodies. A typical ELISA assay takes longer than 2 hours and involves multiple wash and incubation steps. In contrast, the solution based serological assay in this disclosure can be completed within 30 minutes without the need for wash steps.

### 5.4. Combination Assays

In some embodiments, the patient sample is incubated with S sensor and N sensor in a single reaction to determine the presence of the antibodies against the S protein and the antibodies against the N protein. Any of the S sensors and N sensors described above can be used in this combination assay.

### 5.5. Epitope-Mapping/Epitope Masking

In some aspects, it is desirable to characterize antibodies detected in the patient sample, for example, to determine the epitopes recognized by these antibodies. One way to determine the epitopes is by performing a competition assay using a reference antibody. Preferably, the epitopes which the reference antibody is against are known. For example, a reference antibody for the anti-Spike antibodies can be C135 antibody, as described in Robbiani et al., BioRiv, 2020. Methods for determining the epitopes for the antibodies in the patient serum are also substantially described in the Example 5. In brief, an epitope-masked sensor is first constructed by incubating the protein sensor with the reference antibody. The original sensor or the epitope-masked sensor are added to the diluted patient serum for detection-- a decrease in signal with the epitope-masked sensor indicates the presence of the patient antibodies competitive with the reference antibody, i.e., the antibodies bind to the same or overlapping epitopes as the reference antibody. That is to say, detection of a signal from the original sensor that is substantially higher than the signal from the epitope-masked sensor, indicate that the patient antibodies are competitive with the reference antibody. The term “substantially higher,” refers to that a first value is at least 10%, at least 20%, at least 30%, at least 40%, at least 50%, at least 60%, at least 70%, or at least 80% higher than a second value. Antibodies that bind to different epitopes on the viral protein domain may act in synergy to neutralize SARS-CoV-2. Thus, in some cases, antibodies against defined, different epitopes may be used in combination to treat patients infected with SARS-CoV-2 or prevent the infection of SARS-CoV-2.

### 5.6 Neutralization Test

In some embodiments, after positive detection of the antibodies in the patient sample, the patient sample is tested for the neutralization capacity of the antibodies against SARS-CoV-2. Methods for testing neutralization capacity of the serum are known, for example, as disclosed in Robbiani et al., BioRxiv 2020, doi.org/10.1101/2020.05.13.092619, the entire content of which is herein incorporated by reference. In an exemplary assay, the patient serum that has been confirmed to contain antibodies against the SpikeRBD or the N protein is incubated with a SARS-CoV-2 pseudotype virus solution for a time period (e.g., between 20 minutes and 1.5 hours) and at a suitable temperature (e.g., at 37° C.). The mixture can then be incubated with 293 T cells that have been transformed to express the human ACE2 receptor for 48 hours, after which the cells can be washed and analyzed with Luciferase Cell Culture Lysis. The luciferase activity was measured and relative luminescence units obtained were normalized to those derived from cells infected with a SARS-CoV-2 pseudotyped virus in the absence of patient serum or plasma. The half-maximal inhibitory concentration for plasma can be determined using a four-parameter nonlinear regression model. As shown in Example 4 and FIG. 2H, patient samples in which the anti-SpikeRBD antibodies have been detected all showed neutralization capability, and the results correlated strongly with the NT50 of the other anti-SpikeRBD antibodies previously reported (Robbiani et al., 2020).

### 5.7 Saliva Assays

The N sensor and S sensors disclosed herein can be used to detect anti SAR-CoV-2 antibodies in saliva samples from patients. Saliva can be collected using art-known methods. In one aspect of the invention, the patient saliva sample is combined with a diluent prior to combining with a first fusion protein that comprises a first SARS-CoV-2 viral protein domain and a first peptide fragment of a split reporter protein, and a second fusion protein that comprises a second SARS-CoV-2 viral protein domain and a second peptide fragment of the split reporter protein to produce a mixture. Thus, in an embodiment the biological sample used in the assay comprises subject saliva diluted in an aqueous buffer.

As described herein, although a significant reduction in sensitivity was observed when using the assay to detect anti-SARS-CoV-2 antibodies in undiluted saliva relative to buffer alone, no loss in sensitivity when the saliva was diluted 1:2 in PBS buffer. See, FIG. 12. As shown in Table 4C, Out of the 42 samples, 33 had signals above the two healthy saliva controls, indicating a 79% assay sensitivity (Table 4C).

In various embodiments the saliva sample is diluted in an aqueous solution, preferably a buffered aqueous solution, that is compatible with the other assay components (i.e., does not interfere with the assay or its detection). In one example the diluent is phosphate buffered saline (PBS), PBST, or PBSTB. In other embodiments the diluent is normal saline, phosphate buffer, tris-buffer and the like. In certain aspects the buffer may be a zwitterionic buffer (e.g., MOPS or HEPES). In some cases the diluent may be water. Typically the diluted solution has a pH between 6 and 9, e.g., 6 to 8, e.g., 6.5 to 7.5.

Saliva may be diluted (e.g., in PBS) at a ratio of 1:2 (saliva:buffer). In certain embodiments the dilution ratio is in the range from 1:1.5 to 1:10, for example in the range 1:2 to 1:5. In various embodiments the reaction volume is greater than 20 microliters, e.g., 25 to 200 microliters, 50 to 150 microliters or 75 to 125 microliters. In some cases the reaction volume is about 100 microliters.

### 6. Kits

Materials and reagents useful for the diagnostic assays may be provided in kit form, optionally kits in which various reagents are provided in separate vials or containers. Kits may include (i) fusion proteins comprising a vial protein domain (e.g., an SpikeRBD domain or an N protein domain as described herein) and a detection moiety (e.g., a split reporter peptide fragment as described herein) and (ii) detection reagents (e.g., a luciferase substrate). A kit disclosed herein may include N sensor fusion proteins, S sensor proteins, or both S sensor fusion proteins and N sensor proteins. A kit may also contain buffers suitable for antibody-antigen interaction, including, e.g., PBS or TBS. In some embodiments, the buffer contains BSA. The kit may also comprise a negative control sample, which may contain a buffer (e.g., PBST) and 4-10% serum (e.g., fetal bovine serum). In some embodiments, the negative control may be pooled serum samples collected from individuals who have been confirmed as not having SARS-CoV-2 antibodies through other means. The kit may also comprise a positive control, which includes a known SpikeRBD antibodies as described above. Multiple fusion proteins (e.g., an SpikeRBD-SmBiT fusion protein and an SpikeRBD-LgBit fusion protein) may be provided in separate containers or premixed. In one approach a kit may contain a vessel for conducting the reaction, such as an assay plate (such as white plate for luciferase assays). In some embodiments, the kit further comprises a device that is configured to separate serum from whole blood samples from patients.

In some embodiments, the fusion proteins of the protein sensor, assay buffers (e.g., dilution buffers) and/or the detection reagents are supplied in lyophilized form, which are reconstituted to suitable concentrations as described above in solution prior to use.

In some embodiments, the kit may also include instructions to perform one or more of the steps including reconstituting the sensor fusion proteins, contacting the sample with reconstituted sensor proteins, and detecting the signal from the split reporter formed by the complementary split reporter fragments.

### 7. Examples

### 7.1 Example 1. Engineering SpikeRBD Sensors

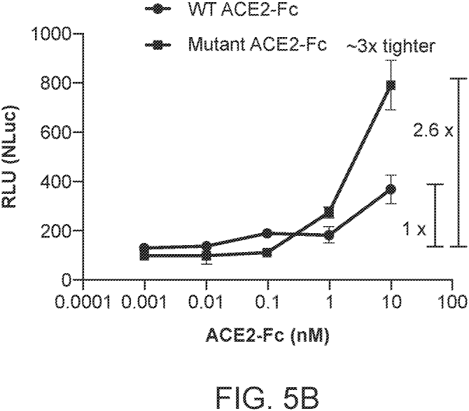

To engineer SpikeRBD sensors (S sensors), we fused NanoLuc fragments to the Spike receptor binding domain (SpikeRBD) [SEQ ID NO:1]. The SpikeRBD was selected because a large proportion of anti-Spike antibodies target the SpikeRBD domain (Amanat et al., 2020; Byrnes et al., 2020). We modeled Abs binding to two epitopes in the SpikeRBD domain to determine the optimal linker length between SpikeRBD sequences and SmBiT/LgBiT. Modeling was based on the assumption that the two fragment binding domains (Fabs) in an immunoglobin (Ig) molecule can span a wide range of distances but centers around 120 Å (FIGS. 4A and 4B) (Sosnick et al, 1992). One of the epitopes is an ACE2-competitive epitope (FIG. 4A), and the other epitope is the binding epitope of CR3022, an ACE2 non-competitive neutralizing patient Ab for SARS-CoV-1 and 2 (FIG. 4B) (Yuan et al., 2020). Our models also estimated that each residue in a flexible linker spans ~2.5A. We engineered SmBiT fused to the C-terminus of SpikeRBD with a 15 amino acid or 25 amino acid (S15, S25) linker, and LgBiT fused to the C-terminus of SpikeRBD with 5, 15, or 25 aa linker (L5, L15 and L25). These fusion proteins were expressed in Expi293 cells and resulted in varied yields (FIG. 1C). The N-terminal fusions to SpikeRBD were not tried because the N and C termini localize in close proximity and we hypothesized this alternative design will result in similar sensitivity.

To determine how the S sensors perform on recombinant SpikeRBD binders, CR3022, an antibody isolated from SARS-CoV-1 patient but cross-reacts with SARS-CoV-2 SpikeRBD, was used to determine the optimal linker lengths, concentrations, and impact of affinities for the S sensors. First, 27, 9, 3, 1, 0.33 and 0.11 nM of the L15 + S25 sensors were mixed with increasing 10x dilutions of recombinant CR3022 (FIG. 1D). After a 20 minute incubation at room temperature (RT), the NanoLuc substrate (Promega) was added for another 10 min and the luminescence signal was read on a plate reader. Interestingly, high concentrations of reporters resulted in stronger background luminescence signal and therefore lower detection sensitivity of CR3022, which is likely due to increased basal association of the two split reporters at increased concentrations. Meanwhile, low concentrations (e.g. 0.33 and 0.1 nM) provided less signal than 1 nM of sensors. Sensors at 1 nM was used for following assays (FIG. 1D).

Furthermore, we queried if linker lengths affect detection sensitivity of CR3022. Interestingly, sensors with varied linker lengths resulted in robust dose-dependent luminescence signal with not much differences in detection sensitivity with the L15 and L25 sensor performed slightly better than L5 sensor (FIG. 5A). These results indicated we selected a proper range of linker lengths. Taking into the various expression yields into consideration, the L15 and S25 sensor pair which expressed at the highest yields (FIG. 1C) were used for further assays.

To determine whether varying binding affinities of the binders to SpikeRBD will affect signal strength, we performed a head-to-head comparison of ACE2-Fc, which is the human ACE2 peptidase domain (aa 18-614) fused to a dimeric human Fc domain (Lui et al., 2020), and an engineered variant which binds tighter to SpikeRBD (K_{D} = 20.4 vs 5.5 nM for wild type and mutant, respectively). A stronger luminescence signal was generated by the mutant ACE2-Fc (FIG. 5B). This observation indicated the sensors will report not only the presence of larger quantity of anti-SpikeRBD binders but also tighter binders. However, binders in the higher affinity range (K_{D} << 1 nM) will be affected less by binding affinities as these high-affinity Abs would likely bind equally well to the S sensors at 1 nM of sensor concentration.

### 7.2 Example 2. S Sensors Bind Multiple Anti-RBD Antibodies That Recognize Different Epitopes

We determined if the S sensors (L25 + S15) can detect patient-derived anti-SARS-CoV-2 Abs. Four SARS-CoV-2 neutralizing Abs were recombinantly expressed. These Abs bind three distinct epitopes on SpikeRBD (FIG. 1E) and potentially neutralize SARS-CoV-2 virus through different mechanisms. CR3022 is an Ab identified from a SARS-CoV-1 patient that cross-reacts with SARS-CoV-2 SpikeRBD and binds at a cryptic site in full-length Spike outside of the ACE2-binding site {Yuan et al., 2020}. C004, C105 and C135 were identified from SARS-CoV-2 patients {Robbiani et al., 2020). While C004 and C105 both compete with ACE2-Fc for binding SpikeRBD, C135 does not compete with C004, C105, CR3022 or ACE2-Fc. See, FIG. 1E. Thus the epitope bound by C135 recognizes a unique binding epitope on SpikeRBD. Interestingly, all four antibodies, albeit binding to various epitopes, all generated dose-dependent luminescence signal (FIG. 1F). The signal strength varied by 1-3 fold for different Abs across varying concentrations. CR3022 generated the strongest luminescence signal. This is consistent with our protein models showing CR3022 binding positions the LgBiT/SmBiT domains in an orientation that is optimal for enzyme reconstitution (FIGS. 4A and 4B). These results highlighted that the S sensors can detect patient Abs that bind to various epitopes on SpikeRBD with slightly varied detection sensitivity.

### 7.3 Example 3. Antibody Competition Assay

We next adapted this solution Ab-detection assay to incorporate an Ab competition condition where patient Igs compete in the presence of recombinant patient Fabs for binding to SpikeRBD. We first generated Fabs either by papain treatment of IgGs or by recombinant expression. Next, “epitope-masked” sensors were prepared by incubating 1 nM sensors with 1 µM Fabs at RT for 15 min. The “non-masked” or “epitope-masked” sensors were then added to 10 nM of the IgGs.

As expected, C004 and C105 Fab competed to ~90% for the C004 and C105 IgGs, while C135 did not. Similarly for C135 IgG and CR3022, the signal was competed ~90% by the corresponding Fabs, while the addition of other Fabs did not reduce signal to such extent. Interestingly, in one case for C135 IgG, the addition of cleaved CR3022 Fab resulted in an increase in signal from the C135 IgG alone. We hypothesize that binding of this Fab may alter the conformation of the Spike-RBD to allow better binding for C135 IgG. This result provided proof-of-concept evidence showing the “epitope-masked” sensors can be adapted to determine the presence of defined epitope classes of patient anti-RBD Abs and use antibodies against different epitopes in synergy to neutralize SARS-CoV-2.

### 7.4 Example 4. Engineering and Characterization of SARS-CoV-2 N Protein Sensors

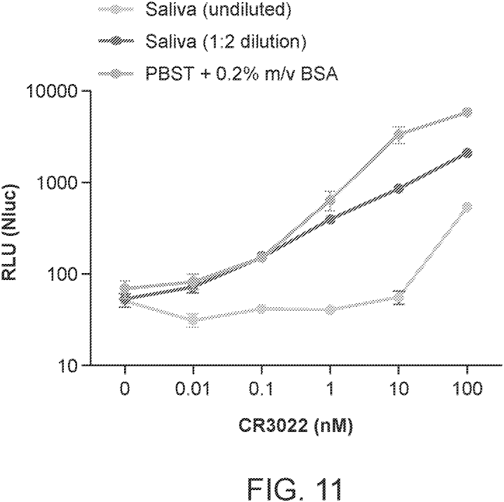

To design a N sensor, we studied the domain geometry and crystal structures of the N protein (FIGS. 1I and 1J). N protein has a C-terminal dimerization domain (aa 258-419) (FIG. 11) which was not used in our N sensor designs because the dimerization of N protein sensors may lead to high basal NanoLuc reconstitution levels. In addition, the N-terminal sequences (aa 134-171, 153-190, and 210-247) are more immunogenic than the C-terminal sequences {Zamecnik et al., 2020, #94100}. Furthermore, while the crystal structure of domain aa 181-257 is not available, the atomic structures of aa 44-180 (the N-terminal RNA binding domain, PDB 6VYO 2OFZ, 6YI3, available at www.rcsb.org/pdb/static.do?p=general_information/ about_pdb /index.html) showed that the N and C termini are located far from each other. We reasoned that fusion at N or C termini may result in different sensor sensitivity (FIG. 1J). Given this knowledge, two fusion sensor pairs were designed:
(1) LgBiT/SmBiT-N(aa 44-257)
(2) N(aa 44-180)-LgBiT/SmBiT. (SEQ ID NO: 10/11)
A 10-aa GS linker (SEQ ID NO:34: GGGTSGGGGS) was inserted between the NanoLuc domains and the N protein domains. Sensors (1) and (2) were expressed in 293Expi cells and resulted in modest protein yields (FIG. 1J).

### 7.5 Example 5. Detecting SARS-CoV-2 Antibodies Using SpikeRBD Sensors and N Sensors

Subsequently, we interrogated if the S and N sensors can enable us to establish a simple and rapid COVID-19 serological test. We first used a small set of sera samples collected 14 or more days following resolution of COVID-19 symptoms from convalescent patients that had RT-PCR-confirmed SARS-CoV-2 infection (FIGS. 2A-C). Healthy control sera were collected before the emergence of SARS-CoV-2 virus. Serial dilutions in PBS+0.05% Tween 20 + 3% BSA (1:12.5, 1:25, and 1:50) of heat-inactivated (56° C., 60 min) sera in 10 µl volume were added to 384-well white plate. Next, 10 µl of the mixture of 2 nM S or N sensors (final concentration 1 nM were added to the diluted sera samples. The reactions were incubated at RT for 20 min, followed by the addition of the NanoLuc substrate and another incubation at RT for 10 minutes. Finally, luminescence signal was detected with a plate reader.

Excitingly, robust dose-dependent luminescence signal was observed using both the S and the N sensors (FIGS. 2A-C). The S sensor generated signal for all five patients tested. The LC and SC sensors (N(aa 44-180)-LgBiT/SmBiT) detected patient Abs from all four patients tested (FIG. 2B), while the LN and SN sensors (LgBiT/SmBiT-N(aa 44-257)) only detected Abs from two patient sera samples that had the strongest seropositivity (FIG. 2C). The difference in sensitivity of the two N sensor designs suggested the epitope of anti-N antibodies positioned the C-terminal fused LgBiT/SmBiT fragments in an optimal orientation for NanoLuc reconstitution. Interestingly, we observed varying degrees of anti-S and N antibody seropositivity between patients, which is consistent with previous findings of heterogeneous responses of patient sera in serological tests (FIGS. 2A, 2B, and 2C) (Byrnes et al, 2020; Amanat et al., 2020). The same trend of varying degree of seropositivity across patients was observed by anti-S ELISA serological analysis of the same set of sera samples {Byrnes et al., 2020}.

In another experiment, 1 nM SpikeRBD-split sensors were incubated with various dilutions of patient serum and healthy control serum, followed by addition of the luciferase substrate (FIG. 1D). Dose-dependent luminescence signal was observed from patient 1 serum, which also showed the strongest anti-RBD antibody response in a parallel ELISA-based serology experiment.

### 7.6 Example 6. Using Protein Sensors to Detect SARS-CoV-2 Antibodies in an Expanded Patient Cohorts

We next applied this new assay in an expanded patient cohort (FIG. 2D). Similarly, these samples were collected from outpatient individual free of symptoms suggestive of COVID-19 for at least 14 days. The demographics and clinical characteristics of these patients have been previously published {Robbiani et al., 2020, #73438}. In these experiments a single serum dilution factor was used (1:12.5 for S sensors and 1:25 for N sensors). Similar to the observation made in the pilot assay (FIGS. 2B, C), we observed varying degrees of seropositivity for both the S and N sensors (FIG. 2D). The log₁₀(anti-S signal) correlates very well with the anti-SpikeRBD ELISA intensity performed with an anti-Fab-HRP secondary antibody (FIG. 2F). The signal also correlated strongly to IgG but poorly to IgM ELISA intensities, which may be due to the weak affinities of IgM antibodies than IgGs. Interestingly, patients that developed more anti-SpikeRBD Abs did not always develop more anti-N RNA-binding domain protein Abs. The log₁₀ (anti-S signal) correlates with the log₁₀ (anti-N signal) with an R = 0.47. While ~16% of patients did not develop anti-RBD antibodies based on both the S sensor solution test and the anti-S ELISA test (FIGS. 2D, G), 100% of the patients developed anti-N Abs (FIGS. 2D,G).

To determine assay specificity, we performed the tests on a cohort of healthy patient sera controls (FIG. 2D, sample #701-723), seasonal coronavirus patient sera (FIG. 2E, sample #56-67) and flu vaccine sera samples pre-vaccination (FIG. 2E, sample #68-77) and post vaccination FIG. 2E, sample #78-87). The cutoff values of the assay readout for the N sensor and S sensor were determined from 55 healthy individual, seasonal coronavirus and flu vaccine samples (used as reference samples) (Table 1). Although other approaches of determining cutoffs can be used, in this experiment, the cutoff value = mean +3x standard deviation.

TABLE1

Cutoff values for the S and N sensor serological assays from 56 control samples

| | S | N |
|---|---|---|
| Min | 12 | 21.5 |
| Max | 44.5 | 200.5 |
| Median | 23.2 | 76.5 |
| Mean | 24.5 | 83.1 |
| Standard deviation | 7.1 | 46.5 |
| Derived cutoff | 45.9 | 222.5 |

All these samples generated significantly lower luminescent signal than the COVID-19 patient sera samples. Interestingly, seasonal coronavirus patient sera showed slightly elevated N signal comparing to the other controls, which may be due to the protein homology between N proteins from SARS-CoV-2 and other coronaviruses. These results demonstrated a 100% assay specificity.

The inter-assasy, inter-day and intra-day variability of spLUC assay were also evaluated. Forty-six (46) plasma samples were assayed a total of five times in three independent experiments over two days for each sensor. The coefficient of variation was calculated for intra-assay, intra-day, and inter-day variability. Coefficient of variation was calculated as ratio of standard deviation to the average value. The results are shown in Table 2 below. The results indicate that tests run on different days and different assays on the same day are highly consistent, with variations in signal generally less than 10%.

TABLE 2

coefficient of intra-assay, intra-day, and inter-day variation

| | Intra-assay | Intra-day | Inter-day |
|---|---|---|---|
| S sensor | 6% | 7% | 8% |
| N Sensor | 6% | 9% | 9% |

Determining the neutralization potency of patient sera is important for identifying high titer neutralizing convalescent sera for treating acutely ill patients and for facilitating population-based studies to understand what proportion of the exposed or vaccinated population might have protective immunity. However, viral neutralization assays necessitate the culture of pseudo or live viruses. Our analysis showed levels of anti-SpikeRBD antibodies correlated strongly with the half-maximal neutralizing titer (NT50) {Robbiani et al., 2020} at R = 0.72 and levels of anti-N-RBD antibodies correlated with NT50 at R = 0.47 (FIG. 2H). This result indicated studying anti-SpikeRBD seropositivity serves as a general straightforward means to assess the neutralization potential of sera samples.

We next performed the assay on a cohort of hospitalized patients (FIG. 2E). These samples were drawn early in infection. It was observed that a population (~25%) of these patients have low or negative anti-S or anti-N signal. These patients may have not seroconverted. Interestingly, for the remainder 75% seropositive samples, the degree of seropositivity was significantly higher than that of the outpatient samples (FIG. 2D). This was consistent with previous findings that anti-SpikeRBD seropositivity is positively correlated with the severity of symptoms including hospitalization (Robbiani et al., 2020). An alternative hypothesis is that levels of antibodies decrease over time. Longitudinal studies on analyzing the time-dependent seropositivity in convalescent patients is necessary to unveil the dynamics of human anti-SARS-CoV-2 humoral response, which is extremely important for vaccine development. In each cohort there was a good correlation between S and N sensors. No significant differences in signal among age groups: Age 19-39, Age 40-59, and Age 60-75 were observed. In some cases, males showed a slightly higher signal as compared to females, but the difference was not statistically significant.

### 7.7 Example 7 Competition Assay

Given the importance of serological assays to not only identify patients that have past exposure to SARS-CoV-2 infection, but also to determine the properties of anti-SpikeRBD Abs generated by patients, we next adapted the solution assay to incorporate an Ab competition component (FIG. 2I). Due to the limited amount of serum samples, we only performed this assay one recombinant antibody, C135, which has an unconventional neutralizing epitope that is not competing with ACE2 binding.

In this assay, we first prepared “epitope-masked sensors” by incubating 1 nM of S sensors with 1 µM of Fab C135, and then added either the original sensors or “epitope-masked” sensors to the 1:25 diluted patient sera for detection (FIG. 2I). A decreased luminescence signal with the “epitope-masked” sensors will indicate the presence of the patient Abs competitive with the corresponding Fab. We performed this assay on 12 patient sera samples with representative high, medium, and low anti-SpikeRBD antibody levels and discovered that sera 7, 21, 42, 72, 98 and 202 have C135 competitive antibodies (FIG. 2J). Among these samples, patient 72 was the serum source for C135, which provided a validation of this method. Moreover, this result showed this non-ACE2 competitive, neutralizing SpikeRBD epitope of C135 is present in many patient samples. Performing these competitive serology assay with different competitive Fab antibodies in an expanded patient cohort is warranted to further our understanding of the correlation between binding epitopes and clinical outcomes.

Finally, we adapted our assays to meet the clinical needs in remote and low-resources settings. While the current properties of the assays meet most of the assay profile requirements, the sample type, reagent format and device requirement need to be further adapted.. First, in low resource settings, it may be difficult to access negative and positive control serum samples. A series of buffer conditions were tested. PBS + 0.05% Tween-20 (“PBST”), 4-10 % FBS was found to reduce background signal generated by the sensors in the absence of antibodies (FIG. 2K). The observed background signal is close to that observed in negative control serum samples and therefore could serve as a generic negative control for the assay. A series of concentrations of antibody C004 was measured with the S sensors in the PBS + 0.05% Tween-20, 10 % FBS buffer and showed linear dose-dependent signal from 0.1 - 10 nM (FIG. 2L). These recombinant anti-SpikeRBD antibodies (e.g. C004) can serve as positive control in the serology assays.

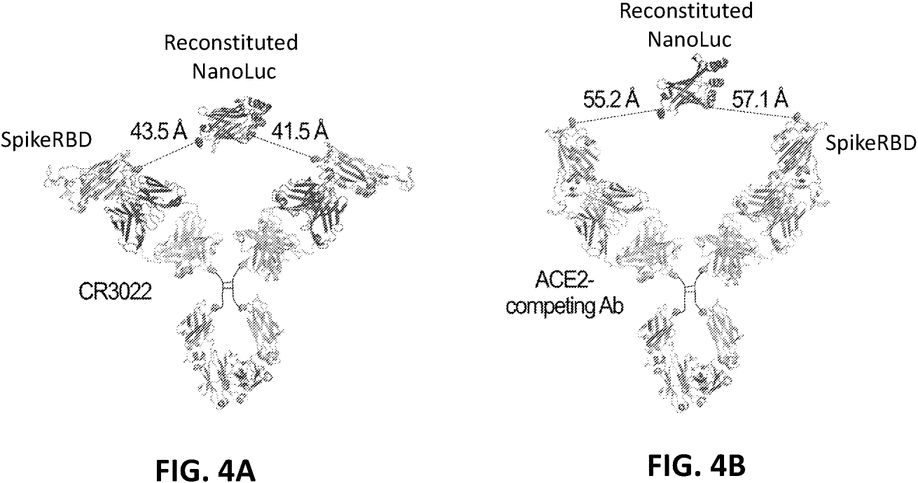

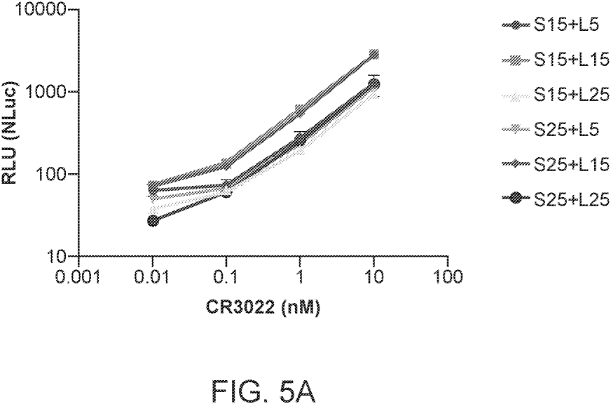

Second, in remote and low-resource areas, it may be challenging to transport reagents at cold temperatures. We tested if the sensors can be lyophilized for ambient temperature storage and transportation. Both S and N sensors, the luciferase substrate and the Nanoluc dilution buffer remained highly functional after lyophilization. Only 0-30% protein in quantity was lost due to the lyophilization process. While in some cases, the signal of S sensors dropped by 50% after lyophilization, the signal of lyophilized N sensors was nearly identical in comparison to the sensors that have never been lyophilized. See FIGS. 3C-3E.

The effect of the lyophilzation on the substrate and dilution buffer were also tested. Reconstituted lyophilized dilution buffer and dilution buffer stored at -20° C. (fresh) also showed similar signal when used to detect recombinant CR3022. The signals of S sensors under conditions in which lyophilizedluciferase substrate and/or lyophilized Nanoluc dilution buffer were used were nearly identical to the signals of Sensors under conditions where fresh luciferase substrate and fresh Nanoluc dilution buffer were used.

Finally, centrifuge access for separating serum from blood samples is another challenge in low-resource settings. We determined if lyophilized sensors can detect antibodies directly from COVID-19 patient blood samples. Blood samples from six patients were collected. 4 out of 6 showed positive anti-S signal and 6 out of 6 showed positive anti-N signal. These results highlighted the potential of the described assays to test patient seropositivity using blood samples. No serological assays were able to detect signal blood samples that have been diluted 12.5x, 25x, and 50x, of which 12.5x being the optimal dilution factor. See FIGS. 3A and 3B.

### 7.8 Example 8 Engineering Split Luminescent Biosensors (spLUC) for SARS-CoV-2 Antibody Detection

S sensors were constructed by fusing the NanoLuc fragments to the receptor binding domain (Spike-RBD), which is the primary target of neutralizing antibodies(Amanat et al., 2020; Byrnes et al., 2020; Okba et al., 2020; Rosado et al., 2020). The N and C termini in the SpikeRBD domain locate in close proximity to each other and fusion of the split enzyme fragments to N or C termini will likely result in similar detection sensitivity (PDB: 6W41). We modeled S-RBD binding to two antibodies, C105 (Robbiani et al., 2020; Barnes et al., 2020), an ACE2-competing binder, and CR3022 (Yuan et al., 2020), an ACE2 non-competing binder, to determine linker lengths. Modeling of ACE2-competitive antibody C105 (PDB: 6XCN) binding to SpikeRBD-SmBiT/LgBiT sensors shows two polypeptide linkers that span ~200 angstrom in between of the Spike-RBD domain and the SmBiT/LgBiT reporters are required. Based on the models, we constructed SmBiT fusions to S-RBD C-terminus with 15 or 25 residue Glycine/Serine (GS) linkers (S15 and S25), and LgBiT fusions to S-RBD C-terminus with 5, 15, or 25 residue GS linkers (L5, L15 and L25). These variants varied in expression yields (Table 5). Using recombinantly expressed S-RBD antibodies and ACE2 variants, we determined the optimal linker variant, enzyme concentration, buffer conditions, and impact of antibody-antigen binding affinity to signal strength (FIGS. 2K and 5B). The (L15+ S25) sensor pair at 1 nM enzyme concentration was identified as the optimal conditions for all subsequent assays.

In further characterizing the relationship between assay signal strength and antibody concentration/binding affinity, we performed ordinary differential equation modeling in R. The modeling predicted a linear relationship between antibody concentration and luciferase signal, consistent with our experimental data (FIG. 8A). In addition, the results highlighted that the sensors at 1 nM are more sensitive to an antibody binder with a K_{D} ≤ 1 nM. Importantly, this threshold is equivalent to the median affinity reported for polyclonal antibody repertoires (Poulsen et al., 2007; Reddy et al., 2015).

To construct the N sensors, we used the N-terminal sequence because aa 44-257 are found to be more immunogenic than the C-terminal dimerization domain (aa 258-419) (Zamecnik et al., 2020). In addition, dimerization promoted by the C-terminal domain may lead to high basal NanoLuc reconstitution levels. The atomic structures of N (aa 44-180) (Kang et al., 2020) showed the N and C termini are not in close proximity and therefore fusion at the N or C terminus may result in different sensor sensitivity. The N and C termini in the Nucleocapsid-RBD domain locate far from each other and fusion of the split enzyme fragments to N or C termini may result in different detection sensitivity (PDB: 6YI3). Given this knowledge, three fusion sensor pairs were designed: (a) LN+SN: L/S-N(aa 44-257), (b) LC+SC: N(aa 44-180)-L/S, and (c) LC2+SC2: N(aa 44-257)-L/S, where L and S represent LgBiT/SmBiT, C represents C-terminal fusion, and N represents N-terminal fusion (Table 7). Testing on commercial polyclonal anti-N protein antibody revealed that the LC + SC and LC2 + SC2 sensors generated stronger signals over LN + SN (Table 8). The LC + SC sensors generated linear, dose-dependent signals with commercial anti-N protein antibody (FIG. 8B).

We next designed a simple and rapid protocol to assay a pilot set of serum samples from convalescent SARS-CoV-2 patients. Two healthy control sera collected before the emergence of SARS-CoV-2 virus were also tested. Serial dilutions (1:12.5, 1:25, and 1:50) of heat-inactivated sera were measured using S or N sensors. Robust, dose-dependent luminescence signal was observed across all serum concentrations tested, with the 12.5-fold dilution showing the highest signal (FIGS. 2B and 2C). The S (L15+S25) sensors generated signal for all five patients tested (FIG. 2B). The N (LC+SC) sensors detected patient antibodies from all four patients tested (FIG. 2C). However, the N (LN+SN) sensors only detected antibodies from two patient sera samples that had the strongest seropositivity (Table 9), which further confirmed a C-terminal fusion enhances NanoLuc reconstitution relative to the N-terminal fusion.

### Competitive spLUC Assay to Profile Epitope-Classes of Antibodies

In addition to a test to determine total binding antibodies, an assay that allows profiling of epitope classes of antibodies can be highly valuable. In this regard, competitive ELISA assays developed by us and others have enabled characterization of percentage of ACE2-competitive antibodies (Byrnes et al., 2020; Tan et al., 2020a). These assays can potentially serve as surrogate viral neutralization tests. However, S-RBD is known to have multiple additional neutralization epitopes outside of the ACE2-binding site. An assay that allows for rapid, unbiased profiling of those alternative epitopes could unveil further details of a patient’s humoral response to neutralize SARS-CoV-2.

We first show that spLUC assay can detect antibodies binding to various epitopes on S-RBD. We expressed and tested four reported neutralizing antibodies which bind to three distinct epitopes on S-RBD. This includes: C004 and C105 (Robbiani et al., 2020), which are ACE2-competitive binders; CR3022 (Yuan et al., 2020), which binds at a cryptic site outside of the ACE2-binding site; and C135 (Robbiani et al., 2020), which does not compete with C004, C105, CR3022 or ACE2-Fc, representing a unique binding epitope on S-RBD. The epitopes of the CR3022, C004, C105, and C135 were characterized using a Biolayer interferometry (BLI) experiment. In these experiments, biotinylated Spike-RBD protein is immobilized on a biosensor. The biosensor is first incubated with one antibody for binding to saturation, followed by incubating with another antibody or ACE2-Fc. The results show that patient antibodies for SARS-CoV-2 have various epitopes on the S-RBD. C004 and C105 have ACE2-competitive epitopes, whereas C135 and CR3022 have non-ACE2-competitive (and non-overlapping) epitopes.. All four IgG antibodies generated dose-dependent luminescence signals at ≥ 0.1 nM concentrations (FIG. 1F).

We then designed a competitive spLUC assay to determine presence of a specific epitope class of antibodies. Out of the four antibodies tested, C135 represents an unconventional and less understood epitope class. It neutralizes very potently (IC50 = 17 ng/ml) and could be potentially used as in combination with other ACE2-competitive binders as a cocktail therapy. We converted C135 IgG to a single binding arm Fab binder, and pre-incubated 1 µM of C135 Fab with the S sensors to generate “blocked sensors”. By comparing signal between the original and the “epitope masked” sensors, we can determine how much signal from a patient’s sample corresponding to antibodies with a similar epitope. We assayed 12 patient serum samples with representative high, medium, and low anti-S-RBD antibody levels at a 1:25 dilution of serum. IgG C135 served as a control for competition with Fab C135. Indeed, the luminescence signal of IgG C135 was reduced by ~90% with the blocked sensors, which provided a validation of this method. Sera 7, 21, 42, 72, 98 and 202 showed a decrease in luminescence signal, indicating they likely have C135-competitive antibodies (FIG. 2J). Patient #72 was the source for identifying C135 (Robbiani et al., 2020) and indeed showed reduction in the spLUC signal when competed with Fab. These results suggested that antibodies recognizing this unconventional, neutralizing S-RBD epitope are present in a significant proportion of patient samples. Performing this competitive serology assay with different competitive Fab antibodies in an expanded patient cohort could further our understanding of the distribution of epitopes on S-RBD as well as the correlation between binding epitopes and clinical outcomes.

### Characterization of Larger Cohorts of Serum/Plasma Samples Using the spLUC Assay

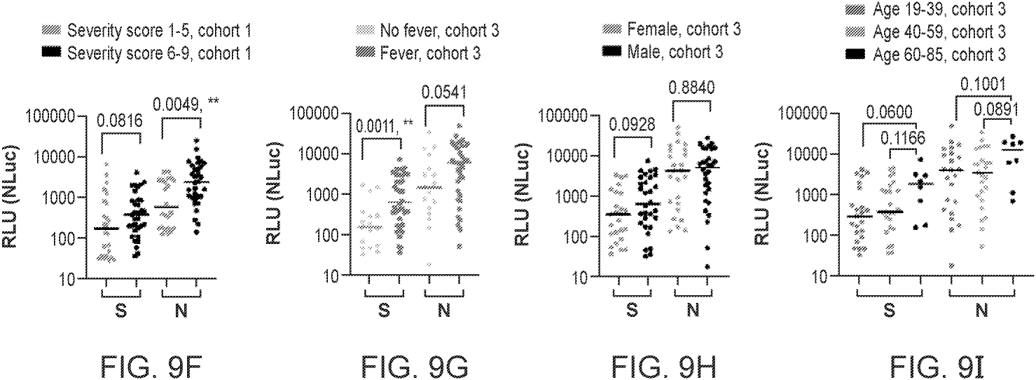

We next applied this new assay in an expanded number of patients (FIGS. 9A-9I). First, to determine assay cutoff values and specificity, which reflects how well an assay performs in a group of disease-negative individuals, we performed the tests on three cohorts of negative control samples (Total n = 144), which include mainly healthy individual samples, 12 seasonal coronavirus patient samples, and 20 flu vaccine pre- and post-vaccination samples. All controls were collected before the SARS-CoV-2 pandemic. These controls generated significantly lower luminescent signals than the COVID-19 patient sera samples (FIGS. 9A, 9B). The range, median, mean and standard deviation values were calculated, and stringent cutoff values were determined by calculating the mean plus three standard deviations (Table 3). With these determined cutoffs, we calculated the specificity of the S sensors (1:12.5 serum dilution) to be 100% (56/56), and the N sensors (1:12.5 serum dilution) to be 99.2% (119/120).

TABLE 3

Cutoff values for the S sensor (56 control samples) and the N sensor serological assays (120 control samples)

| | S | N |
|---|---|---|
| Serum dilutions | 1:12.5 | 1: 12.5 |
| #samples | 56 | 120 |
| Min | 12 | 2.5 |
| Max | 44.5 | 84 |
| Median | 23.2 | 25 |
| Mean | 24.5 | 29.5 |
| Standard deviation | 7.1 | 17.8 |
| Derived cutoff | 45.9 | 83.1 |

We then used the spLUC assay to study three additional cohorts of patient samples (FIGS. 9A, 9B). Cohort 1 is an outpatient cohort recruited at the Rockefeller University Hospital (Robbiani et al., 2020). The samples were collected from individuals free of COVID-19 symptoms for ≥14 days. The S sensors showed 84.2% (48/57) sensitivity, and the N sensors showed 100% (56/56) sensitivity. Cohort 2 samples are consisted of remnant sera from COVID-19 patients within Kaiser Permanente Hospitals of Northern California. These samples were drawn in any phase of infection, including the early acute phase. A subset of these patients, who may have not fully seroconverted at the time of sampling, had lower S sensor or N sensor signals compared to others in the spLUC assays. The sensitivities of the assays were 89% (49/55) for S sensors and 98% (46/47) for N sensors. Cohort 3 patients were part of the LIINC (Long-term Impact of Infection with Novel Coronavirus) study from San Francisco General Hospital and included plasma of a mixture of outpatient and inpatient samples drawn in the convalescent phase of the disease. With the S sensors, we detected antibodies in 94% (44/47) of outpatient samples and 100% (9/9) of inpatient samples. With the N sensors, we detected antibodies in 96% (45/47) of outpatient samples and 100% (9/9) of inpatient samples. For all cohorts, the S and N signals show a strong correlation (FIG. 9C). Consistent with previous findings, we observed varying degrees of anti-S and N antibody seropositivity between patients (FIGS. 9A, B), which reflects a wide range of patient humoral response to this virus (Long et al., 2020; Lynch et al., 2020).

Importantly, we observed strong correlation of spLUC assay results to anti-Fab and anti-IgG S-RBD ELISA signals FIG. 11 (R = 0.43-0.91). A base-10 logarithmic scale conversion was applied to the spLUC assay signals for the correlation analysis to ELISA signals. This nonlinear correlation between the spLUC and ELISA assays is likely due to signal compression in ELISAs at high antibody concentrations (Abcam, ELISA guide). For all cohorts, the S sensor seronegative samples also had very low signals in S-RBD ELISA assays, which confirmed the presence of low levels of anti-S-RBD antibodies in these sub-cohorts of patients. Interestingly, the correlations to IgM signals were much weaker. It is possible that IgM was not sensitively detected by the spLUC assay due to the weaker affinities of the individual binding arms in IgMs (Mäkelä et al., 1970), or that the IgG response dominated the signal in many of the tested patients.

One of the key uses of a highly sensitive serology assay is to grade the quality of convalescent sera to neutralize virus (Krammer and Simon, 2020). In cohort 1, our analysis showed the S sensor signals correlated with the half-maximal neutralizing titers (NT50s) reported by Robbiani et al (FIG. 9D, left panel), which is consistent with previous studies on the relationship between anti-S antibody titers and neutralization potency (Seow et al., 2020; Wajnberg et al., 2020; Amanat et al., 2020; Robbiani et al., 2020). Interestingly, we found that the N sensor signals showed a similar correlation with NT50 (FIG. 9D, right panel). Our results indicate determining either anti-S or anti-N seropositivity is a general means to assess the neutralization potential of sera samples.

To try and gain clinical insights from our results, we analyzed our spLUC data in the context of clinical and demographic features. First, the degree of seropositivity for inpatient samples was significantly higher than that of outpatient samples (FIGS. 9A, B, E). Disease severity scores and fever were also associated with a stronger antibody response (FIGS. 2F and G). These results indicated a direct correlation of disease severity and adaptive immune response consistent with previous studies (Zhao et al., 2020; Robbiani et al., 2020; Cervia et al., 2020; Lynch et al., 2020; Long et al., 2020; Seow et al., 2020; Klein et al., 2020). In addition, males had slightly higher antibody titer than females in both cohort 1 and 3 especially for anti-S antibodies, although the differences were not statistically significant (FIG. 9H). This finding was consistent with studies by Klein et al (Klein et al., 2020) and Robbiani et al (Robbiani et al., 2020), but different from Zeng et al (Zeng et al., 2020), which reported females with severe disease developed more antibodies than men with severe disease. This difference might be due to differing selection criteria of patient cohorts. Lastly, patients of age 60-85 showed a higher trend of antibody response compared to those in the 19-39 and 40-59 age brackets, but the difference was not statistically significant (FIG. 9I). Similar findings on the impact of age have been reported previously (Whitman et al., 2020; Lassaunière et al., 2020). These results highlight that demographic and clinical features affect the antibody response of COVID-19 patients. A longer-term, systematic, and population-level serological analysis is needed to further illuminate the variables that affect patient humoral response to SARS-CoV-2.

Collectively, our assay showed high sensitivity and specificity for all three representative cohorts of serum/plasma samples (inpatient, outpatient, acute phase, convalescent phase), with an overall specificity of 100% (S sensor) and 99% (N sensor), and sensitivity of 89% (S sensor) and 98% (N sensor). These values are comparable or superior to reported values for laboratory ELISA and lateral flow tests (Whitman et al., 2020; Lassaunière et al., 2020).

### Adapting the Assay for Low-Resource Settings and Expanded Sample Types

Lastly, we adapted our assays to begin to meet the clinical needs in remote and low-resources settings and for point-of-care or large-scale deployment. While the current properties of the assay meet most of the requirements for deployment in these types of settings, we tested to see if the reaction time (30 minutes), reagent format (frozen aliquots of sensors), and sample type (serum/plasma) could be further optimized.

We first tested if our initial reaction times (20-minute sensor/antibody incubation and 10-minute incubation with substrate) are necessary and optimal. CR3022 (10 nM) was incubated with 1 nM S sensors for 5, 10, 15, and 20 min, followed by luciferase substrate addition and incubation for 0, 2, 4, 6, 8, and 10 minutes (Table 4). All time points resulted in bright luminescence signal, suggesting that the assay could be completed in as short as 5 minutes.

We then tested if the sensors can be lyophilized for ambient temperature storage and transportation. Although a small quantity (0-30%) of S sensors and N sensors were lost due to the lyophilization process (Table 10A), both the lyophilized S and the N sensors can still robustly detect recombinant IgG or patient antibodies in serum with similar sensitivities seen for the fresh sensors (Table 10B, C). Finally, we sought to determine if the spLUC assay could be compatible with other sample types.

First, whole blood samples were collected from six convalescent COVID-19 patients and plasma samples were prepared in parallel for comparison (Table 4B). Remarkably, although the overall signals were lower from whole blood samples, all six samples generated N sensor signals and four had S sensor signals above control levels with the lyophilized sensors (Table 4A). In comparison, all six patients generated N sensor signals and five had S sensor signals above cutoff values from the plasma samples. Strong correlations were observed between the whole blood signals and the plasma signals (R > 0.9). Fresh and lyophilized sensors showed very little difference in performance.

Next, we tested the potential of using saliva as an input. To determine conditions, we added varying concentrations of the CR3022 antibody into saliva from a healthy individual (FIG. 11). We saw a significant reduction in sensitivity for undiluted saliva relative to buffer alone, but remarkably no loss in sensitivity when the saliva was diluted 1:2 in PBS buffer. We then tested 42 saliva samples at 1:2 dilution with the S sensors. We increased the reaction volume from 20 to 100 µl and the luminescence signal integration time from 1000 ms to 5000 ms for better sensitivity, as lower antibody concentrations are expected from saliva samples (Randad et al., 2020). Out of the 42 samples, 33 had signals above the two healthy saliva controls, indicating a 79% assay sensitivity (Table 4C). A moderate correlation of saliva signal with corresponding serum signals was observed (R = 0.66), consistent with recent reports (Faustini et al., 2020). These results highlight the potential of using lyophilized sensors and whole blood or saliva samples as a convenient diagnostic workflow for rapid and quantitative point-of-care antibody testing amenable to broad population deployment or applications in resource-limited areas.

### Plasmid Construction

Plasmids were constructed by standard molecular biology methods. The DNA fragments of Spike-.RBO, N protein, ACE2, and LgBiT were synthesized by IDT Technologies. The SmBiT tag was generated by overlap-extension PCR. The Spike-RBD-5/15/25aa-LgBiT-12xHisTag, Spike-RBD-15/25aa-SmBiT-12xHisTag, N protein(44-180)-10aa-LgBiT-12xHisTag, N protein(44-180)-10aa-SmBiT-12xHisTag, LgBiT-10aa-N protein(44-257)-12xHisTag, and SmBiT-10aa-N protein(44-257)-12xHisTag were generated by subcloning into a pFUSE-12xHisTag vector (adapted from the pFUSE-higG1-Fc vector from InvivoGen). The ACE2-Fc fusion plasmids were generated by subcloning the gene fragments of ACE2 and mutant into the pFUSE-hIgG1-Fc vector. The C004, C105, and C135 IgGs LC and HC plasmids were a generous gift from the Nussenzweig lab (Rockefeller University). The CR3022 IgG plasmids were a generous gift from the Kim lab (Stanford) and the Wilson lab (Scripps). The C135 Fab was cloned by removing the Fc domain from the HC plasmid. Complete plasmid sequences are available upon request.

### Expression and Protein Purification

All proteins were expressed and purified from Expi293 BirA cells according to established protocol from the manufacturer (Thermo Fisher Scientific). Briefly, 30 µg of pFUSE (InvivoGen) vector encoding the protein of interest was transiently transfected into 75 million Expi293 BirA cells using the Expifectamine kit (Thermo Fischer Scientific). For the IgG and Fab proteins, 15 µg of each chain was transfected. Enhancer was added 20 h after transfection. Cells were incubated for a total of 3 d at 37° C. in an 8% CO₂ environment before the supernatants were harvested by centrifugation. Fc-fusion proteins were purified by Protein A affinity chromatography and His-tagged proteins were purified by Ni-NTA affinity chromatography. Purity and integrity were assessed by SDS/PAGE. Purified protein was buffer exchanged into PBS and stored at -80° C. in aliquots.

### Solution Serology Protocol for in Vitro, Serum, Blood, and Saliva Samples

LgBiT and SmBiT sensors for either the Spike or N protein were prepared at a final concentration of each sensor at 2 nM in PBS + 0.05% Tween-20 + 0.2% BSA (PBSTB). For in vitro IgGs or ACE2-Fc, the samples were prepared at 1:10 dilutions in PBSTB unless otherwise specified. Serum and blood samples were diluted to 1:12.5 for both the S and N sensor samples in PBSTB unless otherwise specified. Healthy individual saliva was spiked in with CR3022 and used undiluted or diluted 1:2 in PBSTB. 10 µL of the 2 nM sensor mix and 10 µL of the sample were combined in a 384 Lumitrac white plate (Greiner), skipping every other well and row to avoid potential bleedover in signal. The plate was mixed on a plate shaker for 20 minutes. NanoLuc substrate was diluted according to protocol 1:50 in NanoLuc dilution buffer (Promega) and 15 µL was added to each well, followed by a 10-minute incubation period for the signal to stabilize. Luminescence was measured on a Tecan M200 infinite plate reader with an integration time of 1000 ms.

### Competition Serology Protocol for in Vitro and Serum Samples

The competition serology assay was performed similarly to the solution serology assay except that the S sensors were individually preincubated at 4 nM with 4 µM of either C004 Fab, C105 Fab, or C135 Fab for the in vitro competition assay and C135 Fab only for the serum competition assay. The two sensors + Fab were combined 1:1 to make a 2 nM mix, and 10 µL of this mix was added to the assay as described above.

### Epitope Binning Experiment

Biolayer interferometry data was measured using an Octet RED384 (ForteBio). Biotinylated Spike RBD protein was immobilized on the streptavidin (SA) biosensor (ForteBio). After blocking with biotin, the sensor was loaded with one IgG followed by another IgG or ACE2-Fc to determine epitope binning. PBS with 0.05% Tween-20 and 0.2% BSA was used for all diluents and buffers.

### Spike Protein ELISA Assay

The Spike ELISA assay was performed as previously described. Briefly, 384 Maxisorp plates were coated with 100 µL of 0.5 µg/mL Neutravidin for 1 hr. The plate was washed 3 times with PBS + 0.05% Tween-20 (PBST) followed by incubation with 20 nM S-RBD for 30 minutes. Following 3 washes, the plate was blocked with 3% non-fat milk in PBS for 1 hour. The plate was washed 3 times before the addition of 1:50 dilutions of serum in 1% non-fat milk for 1 hour. After 3 washes, secondary anti-Fab, anti-lgG, or anti-lgM antibody was added and incubated for 30 minutes before the addition of TMB for 3 minutes. The reaction was quenched with 1 M phosphoric acid and absorbance was read on a Tecan M200 infinite plate reader at 450 nm.

### Lyophilization of Sensors

The S and N protein sensors were flash frozen in liquid nitrogen at concentrations between 10-60 µM in 10 µL. A small hole was poked into the caps of the samples and left on a Benchtop K (VirTis) lyophilizer overnight. The next day the sensors were reconstituted in 10 µL of ddH₂O and concentration was verified by nanodrop.

### Serum, Plasma, Whole Blood, and Saliva Samples

The initial small patient cohort was a generous gift from the Wilson lab (UCSF) and heat inactivated at 56° C. for 1 hour before storage at -80° C. The first (outpatient) sample serum set (cohort 1) was a generous gift for the Wilson lab (UCSF) and Nussenzweig lab (Rockefeller). These samples were heat inactivated at 56° C. for 1 hour and stored at 4° C. in a 1:1 dilution in 40% glycerol, 40 mM HEPES (pH 7.3), 0.04% NaN₃, in PBS. The second (inpatient) sample serum set (cohort 2) was a generous gift from the T. Wang lab (Stanford) and were stored at -80° C. as pure serum samples. The third plasma cohort (cohort 3) and blood samples were generous gifts from the Greenhouse lab (UCSF) and Henrich Lab (UCSF) as part of the LIINC study. The plasma samples were stored at 4° C. in a 1:1 dilution in 40% glycerol, 40 mM HEPES (pH 7.3), 0.04% NaN₃, in PBS. The whole blood was stored undiluted at 4° C. Healthy blood samples were purchased from Vitalent and stored undiluted at 4° C. The saliva samples were obtained unstimulated, unexpectorated saliva and were stored at -80° C. Before assayed, the samples were thawed and centrifuged at 9,000 g to remove any insoluble or coagulated matter. Control saliva from November 2019 was purchased from Lee Biosciences, stored at -20° C., and processed similarly.

### Study Approval of Patient Samples

All patient samples were obtained using protocols approved by the Institutional Review Boards (IRB) and in accordance with the Declaration of Helsinki. Samples were de-identified prior to delivery to the lab where all assays described here were performed. Cohort 1 samples were a kind gift of the Michel Nussenzweig, Marina Caskey, and Christian Gaebler of Rockefeller University, collected with Rockefeller IRB protocol DRO-1006. Cohort 2 samples from Kaiser Permanente were collected with Stanford University IRB protocol #55718. Cohort 3 samples were collected with University of California, San Francisco IRB protocol #20-30479. Influenza virus vaccination samples were from a US cohort enrolled at the Rockefeller University Hospital in New York City in 2012-2013 under a protocol approved by the IRB of Rockefeller University (protocol #TWA-0804). Samples from people with seasonal coronavirus infections were collected at the University of Chicago. Samples were de-identified serums of healthcare workers that had respiratory illnesses, were swabbed, and tested positive for common cold coronavirus infections in 2019 (U. Chicago protocol # 09-043-A).

### Data and Statistical Analysis

All graphing and statistical analysis was performed in GraphPad Prism. The non-parametric Spearman correlation analysis was used in Prism to determine the correlation R value between datasets. An unpaired Mann-Whitney test was performed to determine the difference between datasets. A two-tail P value was used to determine statistical significance for all analysis. P < 0.05 was considered statistically significant.

### S Sensor Engineering and Characterization

### Linker Modeling

We modeled S-RBD binding to two antibodies to determine the optimal linker lengths between the S-RBD domains and the SmBiT/LgBiT fusions. The antibody C105 is an ACE2-competitive binder (Robbiani et al., 2020; Barnes et al., 2020), while the antibody CR3022 does not compete with ACE2 (Yuan et al., 2020). Modeling of ACE2-competitive antibody C105 (PDB: 6XCN) binding to Spike- RBD-SmBiT/LgBiT sensors showed that two polypeptide linkers that span ^{~}200 angstrom in between of the Spike-RBD domain and the SmBiT/LgBiT reporters are required. Modeling of ACE2-competitive antibody CR3022 (PDB: 6W41) binding toSpike-RBD-SmBiT/LgBiT sensors shows two polypeptide linkers that span ^{~}90 angstrom in between of the Spike-RBD domain and the SmBiT/LgBiT reporters are required. Based on the assumption that the wing-span of antigen binding sites between Fab arms on a flexible-hinge region of an Fc are roughly ^{~}117-134 Å apart (Sosnick et al., 1992), and residue-to-residue distance in a linker lies between the length of tightly packed alpha-helix residues (1.5 Å) and extended beta-strand residues (3.5 Å), we estimated the total number of linker residues should be ^{~}30-80 amino acids. Antibodies binding to the CR3022 epitope may require a shorter linker for NanoLuc reconstitution than antibodies competitive with ACE2. Considering S-RBD has a C-terminal 15-residue loop to function as part of the linker, we constructed SmBiT fusions to S-RBD C-terminus with 15 or 25 residue Glycine/Serine (GS) linkers (S15 and S25), and LgBiT fusions to S-RBD C-terminus with 5, 15, or 25 residue GS linkers (L5, L15 and L25). These linker variants were expressed in Expi293 cells and varied in expression yields (Table 5). The N-terminal fusions to S-RBD were not designed because the N and C termini localize in close proximity and we hypothesized this alternative fusion design would result in similar sensor performance as the C-terminal fusions. The N and C termini in the Spike-RBD domain locate in close proximity to each other and fusion of the split enzyme fragments to N or C termini will likely result in similar detection sensitivity (PDB: 6W41).

### Optimization of Enzyme Concentrations, Linkers and Buffer Conditions

We then determined the optimal enzyme concentration. A three-fold dilution series from 27 to 0.11 nM of the L15 + S25 sensors were mixed with increasing 10-fold dilutions of recombinant CR3022 (FIG. 1D). After a 20-minute incubation, the NanoLuc substrate was added and allowed to develop for 10 minutes before luminescence signal was read. High sensor concentrations (27, 9, 3 nM) resulted in stronger background luminescence signal and therefore lower detection sensitivity of CR3022, due to increased basal association of the two split sensors. Meanwhile, low sensor concentrations (0.33 and 0.1 nM) generated overall less signal than 1 nM sensors because fewer sensors are captured on each antibody. As a result, sensors at 1 nM were used in all subsequent assays.

Next we queried if linker lengths affect detection sensitivity. Sensors with varied linker lengths were mixed with 10-fold dilutions of CR3022 and all resulted in dose-dependent luminescence signals (FIG. 1D). Little difference in detection sensitivity was observed, except that the (L5 + S15) and (L5 + S25) linker combinations resulted in slightly decreased sensitivity at low antibody concentrations. This result indicated that we had selected a proper range of linker lengths. Based on robust signal and expression yields (Table 5), we chose the L15 and S25 sensor pair for subsequent assays.

Interestingly, we observed that the regular PBSTB assay buffer (PBS, 0.05% Tween-20, 0.2% m/v BSA, PBSTB) produced a higher background signal (average relative luciferase units (RLU) = 70-80) than in serum samples (RLU = 24.5). We tested if supplementing Fetal Bovine Serum (FBS) can reduce background. PBS + 0.05% Tween-20 (PBST) with 4-10 % FBS was found to reduce the signal (mean RLU = 21) to a level that is close to signal from 12.5% serum, and therefore can serve as a proper negative control. Both the recombinant anti-S antibody C004 and the commercial anti-N antibody (Sino biological, Cat#40588-T62-50) produced linear dose-dependent signal in this buffer (FIGS. 8A and 8B), which can be used to generate standard curves and calibrate the instruments for the spLUC assay.

### Impact of Binding Affinities

To determine whether the affinity of the target binding to S-RBD affects signal strength, we turned to two dimeric ACE2 constructs: ACE2-Fc, which is the human ACE2 peptidase domain fused to IgG1 Fc (Lui et al., 2020), and an engineered ACE2-Fc variant that binds ^{~}10x tighter to S-RBD (FIG. 5B). Overall, signal from wild-type ACE2-Fc (K_{D} = 10 nM) is weak, with signal that is more than two standard deviations above background only detected at the highest tested ACE2-Fc concentration (10 nM). Conversely, the enhanced-affinity ACE2-Fc variant (K_{D} = 1 nM) generated a dose-dependent signal from 0.1-10 nM protein concentrations and exhibited 2.6-fold higher signal observed at 10 nM relative to the wild-type ACE2-Fc. These findings indicated the sensors report the presence of not only larger quantities of anti-S-RBD binders but also higher-affinity binders. This property of the sensors suggested spLUC assay may be used to characterize binding affinities of S-RBD antibodies or ACE2 variants for therapeutic applications.

### 7.9 Example 9 Assay Using Whole-Blood From Vaccinated Subjects

Finger-prick whole blood samples (5 µl) from patients who received Pfizer/bioNtech or Moderna SARS-CoV-2 vaccines (which are S protein based vaccines) were collected. Each blood sample was diluted 12.5 fold in PBS+0.05% Tween + 0.2% BSA buffer and detected with 1 nM S or N sensors. The results are shown in Table 12. The results demonstrate that most individuals who received the vaccines developed strong antibody response to the Spike-RBD protein. In addition, as expected, no N protein antibodies were detected. These data highlight that this system can be used in a point-of-care setting with whole blood finger-prick samples as a very convenient sample type.

### 7.10 Example 10 Testing the Variants

In this study, Spike-RBD sensor variants were constructed by fusing the spike RBD sequences from a COVID-19 variant, the UK variant or the SA variant, to the smBiT and LgBiT via linkers to produce the UK Spike-RBD sensor variant or the SA S senor variant. The UK S sensor variant comprises SEQ ID NO: 37 and SEQ ID NO: 38. The SA S sensor variant comprises SEQ ID NO: 40 and SEQ ID NO: 41. CR3022 antibodies at various concentrations were incubated with the Spike-RBD sensor variants. The results show that the Spike-RBD sensors with the mutations in the UK variant (“UK”) and the mutations in the South Africa variant (“SA”) resulted in similar sensitivity in detecting CR3022 compared to the original Spike-RBD sensor (“RBD”). However, different background signals were observed for the three sensors, potentially due to inherent biophysical properties of the sensors. This result highlights that the engineered S sensor variants can successfully detect an anti-RBD antibody.

Next, these Spike-RBD sensor variants were used to test patents who have recovered from COVID-19 infection. 5 uL of serum sample from convalescent patients were diluted 12.5 fold in PBS + 0.05% Tween + 0.2% BSA and were incubated with 1 nM Spike-RBD variant sensors. The results showed that the UK and the South Africa (SA) mutations resulted in similar signal strengths in detecting patient antibodies compared to the original S sensor (“RBD”). The signals were normalized by dividing with the background signals. This result suggests that the antibodies developed in SARS-CoV-2 patients (infected with the original strain) react to similar extent with the different sensor variants built based on recently emerged viral strains.

FIG. 12 shows the strong correlation of the assay signals with the original Spike-RBD sensor (X axis) and the assay signals with the Spike-RBD variant sensors and a new Spike-NTD sensor (Y axis) for convalescent SARS-CoV-2 patient samples. This result further shows the S sensor variants behave robustly with patient samples. It also shows a new sensor design, engineered based the N-terminal domain (instead of the RBD domain) of the S protein (labeled NTD in the graph), can detect anti-Spike-NTD antibodies in convalescent patient serum samples.

### 9. Reference List

All publications, patents, and patent applications cited in this section and elsewhere herein are hereby incorporated by reference.

Adachi et al. (1986). Production of acquired immunodeficiency syndrome-associated retrovirus in human and nonhuman cells transfected with an infectious molecular clone. Journal of Virology, 59(2), 284-291. doi.org/10.1128/jvi.59.2.284-291.1986

Amanat et al. (2020). A serological assay to detect SARS-CoV-2 seroconversion in humans. MedRxiv. doi.org/10.1101/2020.03.17.20037713

Amanat et al. (2020). A serological assay to detect SARS-CoV-2 seroconversion in humans. Nat Med

Barnes et al. (2020). Structures of human antibodies bound to SARS-CoV-2 spike reveal common epitopes and recurrent features of antibodies. bioRxiv

Byrnes et al. (2020) A SARS-CoV-2 serological assay to determine the presence of blocking antibodies that compete for human ACE2 binding. MedRxiv, doi: doi.org/10.1101/2020.05.27.20114652

Byrnes et al., (2020). A SARS-CoV-2 serological assay to determine the presence of blocking antibodies that compete for human ACE2 binding. medRxiv

Casadevall, A., and Pirofski, L.-a. (2020). The convalescent sera option for containing COVID-19. The Journal of clinical investigation 130, 1545-1548.

Cervia et al., . (2020). Systemic and mucosal antibody secretion specific to SARS-CoV-2 during mild versus severe COVID-19. bioRxiv

Chen et al. (2020). Emerging coronaviruses: Genome structure, replication, and pathogenesis. Journal of Medical Virology, pp. 92:418-423. doi.org/10.1002/jmv.25681

Czajkowsky et al. (2012). Fc-fusion proteins: New developments and future perspectives. EMBO Molecular Medicine, 4(10), 1015-1028. doi.org/10.1002/emmm.201201379

Dixon et al.,(2016). NanoLuc Complementation Reporter Optimized for Accurate Measurement of Protein Interactions in Cells. ACS Chem Biol 11, 400-408.

Dixon et al. (2016). NanoLuc Complementation Reporter Optimized for Accurate Measurement of Protein Interactions in Cells. ACS Chemical Biology, 11(2), 400-408. doi.org/10.1021/acschembio.5b00753

Faustin et al., (2020). Detection of antibodies to the SARS-CoV-2 spike glycoprotein in both serum and saliva enhances detection of infection. medRxiv

Fry et al., (2008). Detection of HSV type-1 and type-2 IgG using an in vitro PCA based homogeneous immunoassay. Biochem Biophys Res Commun 372, 542-546.

Gralinski, L. E., & Menachery, V. D. (2020). Return of the coronavirus: 2019-nCoV. Viruses, p. 12(2):135. doi.org/10.3390/v12020135

Gui et al. (2017). Cryo-electron microscopy structures of the SARS-CoV spike glycoprotein reveal a prerequisite conformational state for receptor binding. Cell Research, 27, 119-129. doi.org/10.1038/cr.2016.152

Harcourt et al. (2020). Severe Acute Respiratory Syndrome Coronavirus 2 from Patient with 2019 Novel Coronavirus Disease, United States. Emerging Infectious Diseases, 26(6). https://doi.org/10.3201/eid2606.200516

Hornsby et al. (2015). A High Through-put Platform for Recombinant Antibodies to Folded Proteins. Molecular & Cellular Proteomics, 14(10), 2833-2847. doi.org/10.1074/mcp.0115.052209

Howarth et al. (2008). Monovalent, reduced-size quantum dots for imaging receptors on living cells. Nature Methods, 5(5), 397-399. doi.org/10.1038/nmeth.1206

Kang, et al., (2020). Crystal structure of SARS-CoV-2 nucleocapsid protein RNA binding domain reveals potential unique drug targeting sites. Acta Pharmaceutica Sinica B

Kirchdoerfer et al. (2018). Stabilized coronavirus spikes are resistant to conformational changes induced by receptor recognition or proteolysis. Scientific Reports, 8, 15701. doi.org/10.1038/s41598-018-34171-7

Klein et al. (2020). Sex, age, and hospitalization drive antibody responses in a COVID-19 convalescent plasma donor population. medRxiv

Krammer, F., and Simon, V. (2020). Serology assays to manage COVID-19. Science 368, 1060-1061.

Lan et al. (2020). Structure of the SARS-CoV-2 spike receptor-binding domain bound to the ACE2 receptor. Nature. doi.org/10.1038/s41586-020-2180-5

Lassaunière et al. (2020). Evaluation of nine commercial SARS-CoV-2 immunoassays. Medrxiv

Lei et al. (2020). Potent neutralization of 2019 novel coronavirus by recombinant ACE2-lg. BioRxiv. doi.org/10.1101/2020.02.01.929976

Letko et al. (2020). Functional assessment of cell entry and receptor usage for SARS-CoV-2 and other lineage B betacoronaviruses. Nature microbiology 5, 562-569.

Li et al. (2020). Potential host range of multiple SARS-like coronaviruses and an improved ACE2-Fc variant that is potent against both SARS-CoV-2 and SARS-CoV-1. BioRxiv. doi.org/10.1101/2020.04.10.032342.

Li et al. (2020). Development and clinical application of a rapid IgM-IgG combined antibody test for SARS-CoV-2 infection diagnosis. J Med Virol

Long et al. (2020). Antibody responses to SARS-CoV-2 in patients with COVID-19. Nature medicine 1-4.

Lui et al. (2020) Trimeric SARS-CoV-2 Spike interacts with dimeric ACE2 with limited intra-Spike avidity, bioRxiv preprint doi: doi.org/10.1101/2020.05.21.109157

Lynch et al. (2020). Magnitude and kinetics of anti-SARS-CoV-2 antibody responses and their relationship to disease severity. Clin Infect Dis.

Lui et al. (2020). Trimeric SARS-CoV-2 Spike interacts with dimeric ACE2 with limited intra-Spike avidity. bioRxiv

Mäkelä et al. (1970). Affinity of IgM and IgG antibodies. Immunochemistry 7, 917-932.

Martinko et al. (2018). Targeting RAS-driven human cancer cells with antibodies to upregulated and essential cell-surface proteins. ELife, 7, e31098. doi.org/10.7554/eLife.31098

Monteil et al. (2020). Inhibition of SARS-CoV-2 infections in engineered human tissues using clinical-grade soluble human ACE2. Cell. doi.org/10.1016/j.cell.2020.04.004

Okba et al. (2020). Severe Acute Respiratory Syndrome Coronavirus 2-Specific Antibody Responses in Coronavirus Disease Patients. Emerg Infect Dis 26, 1478-1488.

Peng et al. (2020). Exploring the Binding Mechanism and Accessible Angle of SARS-CoV-2 Spike and ACE2 by Molecular Dynamics Simulation and Free Energy Calculation. Chemrxiv.Org. doi.org/10.26434/chemrxiv.11877492.v1

Poulsen et al. (2007). Kinetic, affinity, and diversity limits of human polyclonal antibody responses against tetanus toxoid. The Journal of Immunology 179, 3841-3850.

Qu et al. (2020). Profile of IgG and IgM antibodies against severe acute respiratory syndrome coronavirus 2 (SARS-CoV-2). Clin Infect Dis

Randad et al. (2020). COVID-19 serology at population scale: SARS-CoV-2-specific antibody responses in saliva. medRxiv

Reddy et al. (2015). Differences in affinity of monoclonal and naturally acquired polyclonal antibodies against Plasmodium falciparum merozoite antigens. BMC microbiology 15, 1-11.

Robbiani et al. (2020). Convergent antibody responses to SARS-CoV-2 in convalescent individuals. Nature

Rogers et al (2020). Isolation of potent SARS-CoV-2 neutralizing antibodies and protection from disease in a small animal model. Science

Rosado et al. (2020). Serological signatures of SARS-CoV-2 infection: Implications for antibody-based diagnostics. medRxiv

Seow et al. (2020). Longitudinal evaluation and decline of antibody responses in SARS-CoV-2 infection. medRxiv

Smith et al. (2020). Immunogenicity of a DNA vaccine candidate for COVID-19. Nat Commun 11, 2601.

Song et al. (2018). Cryo-EM structure of the SARS coronavirus spike glycoprotein in complex with its host cell receptor ACE2. PLoS Pathogens, 14(8), e1007236. doi.org/10.1371/journal.ppat.1007236.

Sosnick et al. (1992). Distances between the antigen-binding sites of three murine antibody subclasses measured using neutron and x-ray scattering, Biochemistry 1992, 31, 6, 1779-1786.

Sosnick et al. (1992). Distances between the antigen-binding sites of three murine antibody subclasses measured using neutron and X-ray scattering. Biochemistry 31, 1779-1786.

Stadlbauer et al. (2020). SARS-CoV-2 Seroconversion in Humans: A Detailed Protocol for a Serological Assay, Antigen Production, and Test Setup. Curr Protoc Microbiol 57, e100.

Tai et al. (2020). Characterization of the receptor-binding domain (RBD) of 2019 novel coronavirus: implication for development of RBD protein as a viral attachment inhibitor and vaccine. Cellular and Molecular Immunology. doi.org/10.1038/s41423-020-0400-4

Tan et al. (2020a). A SARS-CoV-2 surrogate virus neutralization test based on antibody-mediated blockage of ACE2-spike protein-protein interaction. Nat Biotechnol

Tan et al. (2020b). Viral kinetics and antibody responses in patients with COVID-19. MedRxiv

To et al. (2020). Temporal profiles of viral load in posterior oropharyngeal saliva samples and serum antibody responses during infection by SARS-CoV-2: an observational cohort study. The Lancet Infectious Diseases

van Doremalen et al. (2020). ChAdOx1 nCoV-19 vaccination prevents SARS-CoV-2 pneumonia in rhesus macaques. bioRxiv

Wajnberg et al. (2020). SARS-CoV-2 infection induces robust, neutralizing antibody responses that are stable for at least three months. medRxiv

Walls et al. (2020). Structure, Function, and Antigenicity of the SARS-CoV-2 Spike Glycoprotein. Cell, 180, 281-292. doi.org/10.1016/j.cell.2020.02.058

Wang et al. (2020). A human monoclonal 1 antibody blocking SARS-CoV-2 infection. Nature Communications, 11, 2251. doi.org/10.1101/2020.03.11.987958

Whitman et al. (2020). Test performance evaluation of SARS-CoV-2 serological assays. medRxiv

Wrapp et al. (2020). Structural Basis for Potent Neutralization of Betacoronaviruses by Single-domain Camelid Antibodies. BioRxiv. doi.org/10.1101/2020.03.26.010165

Wrapp et al. (2020). Cryo-EM structure of the 2019-nCoV spike in the prefusion conformation. Science, 367(6483), 1260-1263. doi.org/10.1126/science.aax0902

Wu et al. (2020). A new coronavirus associated with human respiratory disease in China. Nature, 579(7798), 265-269. doi.org/10.1038/s41586-020-2008-3

Xiang et al. (2020). Evaluation of Enzyme-Linked Immunoassay and Colloidal Gold-Immunochromatographic Assay Kit for Detection of Novel Coronavirus (SARS-Cov-2) Causing an Outbreak of Pneumonia (COVID-19). MedRxiv

Yan et al. (2020). Structural basis for the recognition of SARS-CoV-2 by full-length human ACE2. Science, 367(6485), 1444-1448. doi.org/10.1126/science.abb2762

Yu et al. (2020). DNA vaccine protection against SARS-CoV-2 in rhesus macaques. Science

Yuan et al. (2020). A highly conserved cryptic epitope in the receptor binding domains of SARS-CoV-2 and SARS-CoV. Science 368, 630-633.

Yuan et al. (2020). A highly conserved cryptic epitope in the receptor-binding domains of SARS-CoV-2 and SARS-CoV. Science, eabb7269. doi.org/10.1126/science.abb7269

Zamecnik et al. (2020). ReScan, a Multiplex Diagnostic Pipeline, Pans Human Sera for SARS-CoV-2 Antigens. medRxiv

Zeng et al. (2020). A comparison study of SARS-CoV-2 IgG antibody between male and female COVID-19 patients: a possible reason underlying different outcome between sex. Journal of Medical Virology

Zhao et al. (2007). Comparison of immunoglobulin G responses to the spike and nucleocapsid proteins of severe acute respiratory syndrome (SARS) coronavirus in patients with SARS. Clin Vaccine Immunol 14, 839-846.

Zhao et al. (2020). Antibody responses to SARS-CoV-2 in patients of novel coronavirus disease 2019. Clin Infect Dis.

### 10. Tables

The values in the tables 4A-4C, 6, 8, 9, 10B-10C, and 11-12 and below represent the luminescence signal from the luciferase.

TABLE 4A

Detecting CR3022 using S sensors

| | Sensor incubation time | | Sensor incubation time | | Sensor incubation time | | Sensor incubation time | |
|---|---|---|---|---|---|---|---|---|
| Substrate Incubation | 20 min | | 10 min | | 5 min | | 15 min | |
| 0 | 3577 | 2883 | 3539 | 4311 | 3866 | 4137 | 3867 | 3351 |
| 2 | 4960 | 2566 | 3301 | 4957 | 4941 | 4950 | 3871 | 3052 |
| 4 | 4245 | 2373 | 2986 | 4373 | 4327 | 4272 | 3580 | 2748 |
| 6 | 3832 | 2283 | 2854 | 3765 | 3918 | 3674 | 3207 | 2525 |
| 8 | 3289 | 2213 | 2493 | 3224 | 3494 | 3325 | 2934 | 2479 |
| 10 | 2931 | 2149 | 2467 | 3104 | 3110 | 3053 | 2834 | 2261 |

TABLE 4B

Detecting anti-SARS-CoV2 antibodies in blood and plams using fresh and lyophilized S sensors

| Plasma | Blood (fresh) | | Blood (lyophilized) | | Plasma | Blood N sensor (fresh) | | Blood N sensor (lyophilized) | |
|---|---|---|---|---|---|---|---|---|---|
| 147 | 38 | 33 | 71 | 54 | 381.5 | 42 | 46 | 61 | 69 |
| 1496.5 | 318 | 307 | 247 | 217 | 4623.5 | 757 | 818 | 603 | 586 |
| 46 | 22 | 40 | 21 | 35 | 888.5 | 143 | 117 | 93 | 112 |
| 442 | 127 | 124 | 104 | 77 | 741 | 153 | 143 | 150 | 116 |
| 1921 | 445 | 441 | 297 | 381 | 17138 | 3765 | 3642 | 3315 | 3235 |
| 31.5 | 9 | 26 | 23 | 33 | 599 | 125 | 105 | 100 | 65 |

TABLE 4C

Detecting anti-SARS-CoV2 antibodies in saliva samples using S sensors

| Sample ID | Serum | | Saliva | |
|---|---|---|---|---|
| 5047 M0 | 4079 | 2625 | 184 | 185 |
| 5048 M0 | 426 | 301 | 57 | 54 |
| 5050 M0 | 1453 | 1139 | 78 | 83 |
| 5065 M0 | 2409 | 1932 | 47 | 49 |
| 5056 M0 | 41 | 32 | 99 | 112 |
| 5057 M0 | 3521 | 3224 | 109 | 82 |
| 5051 M0 | 3902 | 4664 | 400 | 461 |
| 5052 M0 | 329 | 241 | 35 | 36 |
| 5053 M0 | 757 | 669 | 69 | 82 |
| 5063 M0 | 1747 | 1307 | 66 | 47 |
| 5064 M0 | 278 | 222 | 36 | 41 |
| 5065 M0 | 2409 | 1932 | 112 | 149 |
| 5066 M0 | 35 | 30 | 28 | 26 |
| 5068 M0 | 3232 | 2609 | 65 | 81 |
| 5081 M0 | 1456 | 1067 | 44 | 43 |
| 5082 M0 | 2254 | 1857 | 43 | 45 |
| 5061 M0 | 436 | 292 | 41 | 34 |
| 5062 M0 | 1338 | 1051 | 88 | 96 |
| 5072 M0 | 3164 | 2740 | 130 | 110 |
| 5073 M0 | 122 | 117 | 34 | 27 |
| 5075 M0 | 141 | 96 | 47 | 47 |
| 5076 M0 | 45 | 56 | 34 | 35 |
| 5077 M0 | 4984 | 4023 | 168 | 171 |
| 1194 M0 | 202 | 107 | 112 | 110 |
| 5079 M0 | 502 | 377 | 39 | 43 |
| 5080 M0 | 2032 | 1924 | 63 | 95 |
| 5051 M1 | 2994 | 2277 | 90 | 93 |
| 5053 M1 | 794 | 495 | 849 | 1167 |
| 5047 M1 | 3043 | 2378 | 113 | 115 |
| 5048 M1 | 226 | 170 | 33 | 40 |
| 5049 M1 | 11998 | 10919 | 152 | 167 |
| 5045 M1 | 2677 | 2777 | 113 | 122 |
| 5054 M1 | 158 | 143 | 32 | 34 |
| 5036 M1 | 68 | 67 | 87 | 81 |
| 5037 M1 | 889 | 477 | 39 | 48 |
| 5039 M1 | 470 | 307 | 34 | 42 |
| 5027 M1 | 269 | 191 | 55 | 59 |
| 5028 M1 | 488 | 307 | 34 | 24 |
| 5030 M1 | 475 | 338 | 38 | 36 |
| 5031 M1 | 2420 | 1400 | 78 | 76 |
| 5032 M1 | 355 | 278 | 24 | 36 |
| 5042 M1 | 3946 | 2912 | 99 | 120 |

Tables 4A-4C show the luminescence signals from the spLUC assays. Table 4A shows that spLUC assays can be accomplished in as short as 5 minutes. CR3022 (10 nM) was incubated with S sensors for 5, 10, 15, or 20 min. Luciferase substrates were then added and incubated with the reaction mix for 0, 2, 4, 6, 8 or 10 min. All reactions showed bright luminescence signal. Table 4B shows that the spLUC assay is compatible with whole blood samples and show similar signal in the corresponding plasma samples with both fresh and lyophilized sensors (R =321 0.94 for S sensors, R = 1 and 0.98 for N sensor fresh and lyophilized sensors, respectively). Table 4C shows that anti-S antibodies were detected in saliva samples with moderate sensitivity (33/42, 79%). The signals from saliva samples positively correlated with corresponding serum samples (R = 0.66, p< 0.0001). Each reaction was performed in two replicates.

TABLE 5

S sensor yield

| # | Sensor | Yield (mg/L) |
|---|---|---|
| L5 | S(aa 328,5)-5aa-LgBiT | 1.5 |
| L15 | S(aa 328-533)-15aa-LgBIT | 11.1 |
| L25 | S(aa 328-533)-258a-431T | 2.8 |
| S15 | S(aa 328-533}-15aa-SmBIT | 21.4 |
| S25 | S(aa 328-53)-25aa-SmBiT | 26.5 |

TABLE 6

S sensors with varied linker length in detecting CR3022

| CR3022 (nM) | S15 + L5 | | S15 + L15 | | S15 + L25 | | S25 + L5 | | S25 + L15 | | S25 + L25 | |
|---|---|---|---|---|---|---|---|---|---|---|---|---|
| 10 | 1300 | 1306 | 3121 | 2559 | 919 | 968 | 1122 | 1133 | 2864 | 2743 | 1495 | 975 |
| 1 | 309 | 233 | 609 | 619 | 191 | 207 | 197 | 189 | 524 | 578 | 269 | 227 |
| 0.1 | 81 | 63 | 132 | 137 | 64 | 64 | 69 | 69 | 125 | 124 | 64 | 58 |
| 0.01 | 70 | 56 | 74 | 70 | 37 | 37 | 47 | 52 | 68 | 76 | 26 | 28 |

TABLE 7

N sensor yield

| # | Sensor | Yield (mg/L) |
|---|---|---|
| LC | N(aa 44-180)-10aa-LgBiT | 6.4 |
| SC | N(aa 44-180)-10aa-SmBiT | 14.2 |
| LC2 | LgBiT-10aa-N(aa 44-257) | 11.2 |
| SC2 | SmBiT-10aa-N(aa 44-257) | 1.8 |
| LN | LgBiT-10aa-N(aa 44-257) | 1.6 |
| SN | SmBiT-10aa-N(aa 44-257) | 6.8 |

TABLE 8

Design and characterization of N sensors

| Dilution | 44-180 C term | | 44-257 C term | | 44-257 N term | |
|---|---|---|---|---|---|---|
| 100 | 10923 | 10167 | 9061 | 9422 | 1971 | 2202 |
| 1000 | 3997 | 3564 | 2908 | 2790 | 677 | 684 |
| 10000 | 1027 | 694 | 563 | 505 | 118 | 444 |
| 100000 | 811 | 592 | 475 | 327 | 146 | 145 |
| 1000000 | 598 | 600 | 332 | 281 | 138 | 104 |

Table 8 shows that the N-terminal N sensor pair (LN + SN, 44-257) was less sensitive than the LC + SC (44-180) and LC2 + SC2 (44-257) C terminal N sensor pairs when the assay was performed on a rabbit polyclonal anti-N protein antibody (Sino Biological, Cat#: 40588-T62-50).

TABLE 9

Design and characterization of N sensors

| Dilution | Patient 6 | | Patient 8 | | Patient 11 | | Patient 12 | | Control 1 | | Control 2 | |
|---|---|---|---|---|---|---|---|---|---|---|---|---|
| 12.5 | 120 | 105 | 141 | 108 | 50 | 22 | 36 | 20 | 23 | 23 | 27 | 17 |
| 25 | 86 | 77 | 67 | 73 | 15 | 33 | 32 | 20 | 20 | 17 | 13 | 26 |
| 50 | 51 | 72 | 35 | 46 | 20 | 25 | 8 | 20 | 26 | 11 | 30 | 18 |

Table 9 shows that out of the four patients tested, patient 6 and 8 (but not patient 11 and 12) showed signals above controls in the serological assay performed with LN + SN sensors, while all fourpatients showed signals with the LC + SC sensors.

TABLE 10A

characterization of lyophilized N and S sensors

| | | % of recovery | % signal |
|---|---|---|---|
| S sensor | SmBiT | 90 | 50 |
| S sensor | LgBiT | 100 | 50 |
| N sensor | SmBiT | 70 | 100 |
| N sensor | LgBiT | 100 | 100 |

TABLE 10B

Detecting CR3022 using lyophilized S sensors

| | fresh | | | lyophilized | | |
|---|---|---|---|---|---|---|
| No Ab | 98 | 85 | 99 | 62 | 87 | 61 |
| CR3022 0.2 nM | 246 | 193 | 194 | 101 | 111 | 104 |
| CR3022 2 nM | 2802 | 2822 | 2757 | 1226 | 1173 | 1175 |
| CR3022 20 nM | 8009 | 7840 | 7773 | 4845 | 4889 | 4587 |

TABLE 10C

Detecting anti-SARS-CoV-2 antibodies from patient sera

| | Fresh sensor, fresh buffer | | | Fresh sensor, lyophilized buffer | | | Vacuum dried sensor, fresh buffer | | | Vacuum dried sensor, lyophilized buffer | | |
|---|---|---|---|---|---|---|---|---|---|---|---|---|
| No Ab | 56 | 57 | 36 | 35 | 39 | 31 | 20 | 45 | 84 | 17 | 34 | 44 |
| CR3022 0.1 nM | 123 | 96 | 110 | 114 | 116 | 94 | 110 | 168 | 168 | 70 | 164 | 143 |
| CR3022 1 nM | 122 2 | 823 | 724 | 685 | 665 | 689 | 668 | 132 2 | 108 5 | 810 | 103 6 | 928 |
| CR3022 10 nM | 333 3 | 318 0 | 306 5 | 342 4 | 308 4 | 323 4 | 269 6 | 464 0 | 378 8 | 305 1 | 464 6 | 390 2 |

Tables 10A-10C show that the S and N sensors were functional after lyophilization. Table 10A show that both the S and the N sensors can survive lyophilization. The majority of proteins (70-100%) can be reconstituted after lyophilization. The lyophilized S sensors lost 50% of signal. The lyophilized N sensors remain 100% active. Table 10B shows that the lyophilized S sensors detected CR3022 at ^{~}50% signal strength compared to fresh sensors. Table 10C shows that the lyophilized N sensors detected antibodies from patient sera at similar signal strength compared to fresh sensors.

TABLE 11

Detecting CR3022 using Sensors in a portable luminometer

| CR3022 (nM) | Portable luminometer | | Luminescence plate reader |
|---|---|---|---|
| 0 | 172 | 162 | 49 |
| 0.001 | 167 | 165 | 51 |
| 0.01 | 206 | 194 | 65 |
| 0.1 | 522 | 506 | 101 |
| 1 | 2928 | 2838 | 651 |
| 10 | 17646 | 16862 | 3499 |

Table 11 shows that the spLUC assay is also amenable to detection with a Berthold portable luminometer. The handheld luminometer showed similar sensitivity of recombinant CR3022 with S sensors compared to the plate reader. Due to the tube format of the handheld luminometer, the sample volume was doubled and thus the overall signals are higher than for the plate reader samples, but similar sensitivity is maintained. Two technical replicates are plotted from n=1 independent experiment. Lines connecting the means of the samples are plotted.

TABLE 12

Testing finger prick whole-blood samples from vaccinated patients

| S sensor | | N sensor | | S control | | N control | |
|---|---|---|---|---|---|---|---|
| 197 | 163 | 9 | 7 | 1 | 1 | 13 | 16 |
| 64 | 45 | 6 | 5 | 3 | 3 | 24 | 21 |
| 176 | 149 | 7 | 6 | 4 | 3 | 4 | 6 |
| 79 | 70 | 5 | 6 | | | | |
| 203 | 258 | 5 | 7 | | | | |
| 130 | 170 | 16 | 14 | | | | |
| 242 | 269 | 11 | 17 | | | | |
| 28 | 37 | 15 | 15 | | | | |
| 56 | 56 | 14 | 12 | | | | |
| 243 | 188 | 9 | 9 | | | | |
| 25 | 27 | 12 | 11 | | | | |
| 6 | 4 | 11 | 19 | | | | |

The values in the table represent the luminescence signal from NLuc. These results show that most individuals received the S protein based SARS-CoV-2 vaccines developed strong antibody response to the Spike-RBD protein. And the assay can be used to test whole blood finger-prick samples.

TABLE 13

Sequences

| SEQ ID NO | Description |
|---|---|
| 1 | Spike-RBD |
| 2 | ACE2-Fc mutant |
| 3 | SmBiT |
| 4 | LgBiT |
| 5 | N protein RNA binding domain aa 44-180 |
| 6 | N protein aa 44-257 |
| 7 | N protein (aa 258-419) |
| 8 | ACE2 (aa 18-614 of the full length protein) |
| 9 | ACE2-Fc |
| 10 | N(aa 44-180)-LgBiT |
| 11 | N(aa 44-180)-SmBiT |
| 12 | S(aa 328-533)-15aa-lgBiT (L15) |
| 13 | S(aa 328-533)-25aa-SmBiT (S25) |
| 14 | Full length N protein |
| 15 | N protein 134-171 |
| 16 | N protein (aa 153-190) |
| 17 | N protein (aa 210-247) |
| 18 | the L5 linker |
| 19 | the L25 linker |
| 20 | GSSGSS |
| 21 | GGGGGGGG |
| 22 | GSAGSAAGSGEF |
| 23 | the S15 linker |
| 24 | CR3022 IgG LC |
| 25 | CR3022 IgG HC |
| 26 | C004 IgG LC |
| 27 | C004 IgG HC |
| 28 | C105 IgG LC |
| 29 | C105 IgG HC |
| 30 | C135 IgG LC |
| 31 | C135 IgG HC |
| 32 | C135 Fab LC |
| 33 | C135 HC (CH1) |
| 34 | linker |
| 35 | N(aa 44-257)-LgBiT |
| 36 | N(aa 44-257)-SmBiT |
| 37 | RBD UK variant smbit |
| 38 | RBD UK variant Igbit |
| 39 | RBD SA variant smbit |
| 40 | RBD SA variant Igbit |
| 41 | NTD smbit |
| 42 | NTD Igbit |
| 43 | NTD |
| 44 | UK variant RBD |
| 45 | SA variant RBD |

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

The following copending commonly owned patent applications are incorporated by reference in their entirety for all purposes:
ACE2 COMPOSITIONS AND METHODS, Application No. PCT/US______, filed May 11, 2021 (attorney docket number 103182-1244650-005010WO), and
DETECTION ASSAY FOR SARS-COV-2 VIRUS, Application No. PCT/US______, filed May 11, 2021 (attorney docket number 103182-1244642-004910WO).

In the foregoing description, numerous specific details are set forth to provide a more thorough understanding of the present invention. However, it will be apparent to one of skill in the art that the invention described in this disclosure may be practiced without one or more of these specific details. In other instances, well-known features and procedures well known to those skilled in the art have not been described in order to avoid obscuring the invention. Embodiments of the disclosure have been described for illustrative and not restrictive purposes. Although the present invention is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

### 11. Sequences

Spike-RBD [SEQ ID NO:1]

RFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGP
KKSTNL

ACE2-Fc mutant (SEQ ID NO: 2)

QSTIEEQAKTFLDKFNVEAEDLFYQSSLASWNYNTNITEENVQNMNNAGD
KWSAFLKEQSTLAQMYPLQEIQQLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHNEMGNIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYATSS
GGGGENLYFQSSGGGSGGGEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K

SmBiT [SEQ ID NO:3]

VTGYRLFEEIL

LgBiT [SEQ ID NO:4]

VFTLEDFVGDWEQTAAYNLDQVLEQGGVSSLLQNLAVSVTPIQRIVRSGE
NALKIDIHVIIPYEGLSADQMAQIEEVFKVVYPVDDHHFKVILPYGTLVI
DGVTPNMLNYFGRPYEGIAVFDGKKITVTGTLWNGNKIIDERLITPDGSM
LFRVTINS

SEQ ID NO: 5 N protein RNA binding domain aa 44-180

GLPNNTASWFTALTQHGKEDLKFPRGQGVPINTNSSPDDQIGYYRRATRR
IRGGDGKMKDLSPRWYFYYLGTGPEAGLPYGANKDGIIWVATEGALNTPK
DHIGTRNPANNAAIVLQLPQGTTLPKGFYAEGSRGGS

SEQ ID NO: 6 N protein aa 44-257

GLPNNTASWFTALTQHGKEDLKFPRGQGVPINTNSSPDDQIGYYRRATRR
IRGGDGKMKDLSPRWYFYYLGTGPEAGLPYGANKDGIIWVATEGALNTPK
DHIGTRNPANNAAIVLQLPQGTTLPKGFYAEGSRGGSQASSRSSSRSRNS
SRNSTPGSSRGTSPARMAGNGGDAALALLLLDRLNQLESKMSGKGQQQQG
QTVTKKSAAEASKK

SEQ ID NO: 7 N protein (aa 258-419)

PRQKRTATKAYNVTQAFGRRGPEQTQGNFGDQELIRQGTDYKHWPQIAQF
APSASAFFGMSRIGMEVTPSGTWLTYTGAIKLDDKDPNFKDQVILLNKHI
DAYKTFPPTEPKKDKKKKADETQALPQRQKKQQTVTLLPAADLDDFSKQL
QQSMSSADSTQA

SEQ ID NO: 8 ACE2 (aa 18-614 of the full length protein)

QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYA

SEQ ID NO: 9 ACE2-Fc

QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYATSS
GGGGENLYFQSSGGGSGGGEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K

SEQ ID NO: 10 N(aa 44-180) - LgBiT

MRMQLLLLIALSLALVTNSSGLPNNTASWFTALTQHGKEDLKFPRGQGVP
INTNSSPDDQIGYYRRATRRIRGGDGKMKDLSPRWYFYYLGTGPEAGLPY
GANKDGIIWVATEGALNTPKDHIGTRNPANNAAIVLQLPQGTTLPKGFYA
EGSRGGSGGGTSGGGGSVFTLEDFVGDWEQTAAYNLDQVLEQGGVSSLLQ
NLAVSVTPIQRIVRSGENALKIDIHVIIPYEGLSADQMAQIEEVFKVVYP
VDDHHFKVILPYGTLVIDGVTPNMLNYFGRPYEGIAVFDGKKITVTGTLW
NGNKIIDERLITPDGSMLFRVTINSGGGSGHHHHHHHHHHHHGSGLNDIF
EAQKIEWHEG

SEQ ID NO: 11 N(aa 44-180)-SmBiT

MRMQLLLLIALSLALVTNSSGLPNNTASWFTALTQHGKEDLKFPRGQGVP
INTNSSPDDQIGYYRRATRRIRGGDGKMKDLSPRWYFYYLGTGPEAGLPY
GANKDGIIWVATEGALNTPKDHIGTRNPANNAAIVLQLPQGTTLPKGFYA
EGSRGGSGGGTSGGGGSVTGYRLFEEILGGGSGHHHHHHHHHHHHGSGLN
DIFEAQKIEWHEG

SEQ ID NO: 12 S(aa 328-533)-15aa-lgBiT (L15)

MRMQLLLLIALSLALVTNSTSRFPNITNLCPFGEVFNATRFASVYAWNRK
RISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGYQ
PYRVVVLSFELLHAPATVCGPKKSTNLGGGGSGGGGSGGGGSVFTLEDFV GDWEQTAAYNLDQVLEQGGVSSLLQNLAVSVTPIQRIVRSGENALKIDIH VIIPYEGLSADQMAQIEEVFKVVYPVDDHHFKVILPYGTLVIDGVTPNML NYFGRPYEGIAVFDGKKITVTGTLWNGNKIIDERLITPDGSMLFRVTINS
GGGSGHHHHHHHHHHHH

SEQ ID NO: 13 S(aa 328-533)-25aa-SmBiT (S25) (the two underlined regions are two linkers)

MRMQLLLLIALSLALVTNSTSRFPNITNLCPFGEVFNATRFASVYAWNRK
RISNCVADYSVLYNSASEFSTEFKCYGVSPTKLNDLCEFTNVYADSEVIR
GDEVROQIAPGQTGKIADYNYKLPDDETGCVIAWNSNNLDSKVGGNYNYL
YRLFRKSNLKPFERDISTEIYQAGSTPCNGVEGFNCYFPLQSYGFQPTNG
VGYQPYRVVVLSFELLHAPATVCGPKKSTNLGGGGSGGGGSGGGGSGGGG SGGGGSVTGYRLFEEILGGGSGHHHHHHHHHHHH

SEQ ID NO: 14 Full length N protein

MSDNGPQNQRNAPRITFGGPSDSTGSNQNGERSGARSKQRRPQGLPNNTA SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA

SEQ ID NO: 15 N protein 134-171

ATEGALNTPKDHIGTRNPANNAAIVLQLPQGTTLPKGF

SEQ ID NO: 16 N protein (aa 153-190)

NNAAIVLQLPQGTTLPKGFYAEGSRGGSQASSRSSSRS

SEQ ID NO: 17 N protein (aa 210-247)

MAGNGGDAALALLLLDRLNQLESKMSGKGQQQQGQTVT

SEQID NO: 18 (the L5 linker)

GGGGS

SEQ ID NO: 19 (the L25 linker)

GGGGSGGGGSGGGGSGGGGSGGGGS

SEQ ID NO: 20

GSSGSS

SEQ ID NO: 21

GGGGGGGG

SEQ ID NO: 22

GSAGSAAGSGEF

SEQ ID NO: 23 (the S15 linker):

GGGGSGGGGSGGGGS

Sequences for the antibodies: SEQ ID NO: 24 (CR3022 IgG LC)

MGWSCIILFLVATATGVHSDIQLTQSPDSLAVSLGERATINCKSSQSVLY
SSINKNYLAWYQQKPGQPPKLLIYWASTRESGVPDRFSGSGSGTDFTLTI
SSLQAEDVAVYYCQQYYSTPYTFGQGTKVEIKRTVAAPSVFIFPPSDEQL
KSGTASVVCLLNNFYPREAKVQWKVDNALQSGNSQESVTEQDSKDSTYSL
SSTLTLSKADYEKHKVYACEVTHQGLSSPVTKSFNRGEC

SEQ ID NO: 25 (CR3022 IgG HC)

MGWSCIILFLVATATGVHSQMQLVQSGTEVKKPGESLKISCKGSGYGFIT
YWIGWVRQMPGKGLEWMGIIYPGDSETRYSPSFQGQVTISADKSINTAYL
QWSSLKASDTAIYYCAGGSGISTPMDVWGQGTTVTVASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEA
LHNHYTQKSLSLSPGK

SEQ ID NO: 26 (C004 IgG LC)

MGWSCIILFLVATATGVHCAIRMTQSPSSLSASVGDRVTITCQASQDISN
YLNWYQQKPGKAPKLLIYDASNLETGVPSRFSGSGSGTDFTFTISSLQPE
DIATYYCQQYDNLPITFGQGTRLEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC

SEQ ID NO: 27 (C004 IgG HC)

MGWSCIILFLVATATGVHSQVQLVQSGAEVKKPGASVKVSCKASGYTFTG
YYMHWVRQAPGQGLEWMGWINPISGGTNYAQKFQGRVTMTRDTSISTAYM
ELSRLRSDDTAVYYCASPASRGYSGYDHGYYYYMDVWGKGTTVTVSSAST
KGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSC
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGK

SEQ ID NO: 28 (C105 IgG LC)

MGWSCIILFLVATATGSWAQSALTQPPSASGSPGQSVTISCTGTSSDVGG
YKYVSWYQQHPGKAPKLMIYEVSKRPSGVPDRFSGSKSGNTASLTVSGLQ
AEDEADYYCSSYEGSNNFVVFGGGTKLTVLGQPKAAPSVTLFPPSSEELQ
ANKATLVCLISDFYPGAVTVAWKADSSPVKAGVETTTPSKQSNNKYAASS
YLSLTPEQWKSHRSYSCQVTHEGSTVEKTVAPTECS

SEQ ID NO: 29 (C105 IgG HC)

MGWSCIILFLVATATGVHSQVQLVESGGGLIQPGGSLRLSCAASGFTVSS
NYMSWVRQAPGKGLEWVSVIYSGGSTYYADSVKGRFTISRDNSKNTLYLQ
MNSLRAEDTAVYYCARGEGWELPYDYWGQGTLVTVSSASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSLSLSPGK

SEQ ID NO: 30 (C135 IgG LC)

MGWSCIILFLVATATGVHSDIQMTQSPSTLSASVGDRVTITCRASQSISN
WLAWFQQKPGKAPKLLIYEASSLESGVPSRFSGSGSGTEFTLTISSLQPD
DFATYYCQQYNSYPWTFGQGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC

SEQ ID NO: 31 (C135 IgG HC)

MGWSCIILFLVATATGVHSQVQLVESGGGVVQPGRSLRLSCAASGFTFSS
YAMHWVRQAPGKGLEWVAVIPFDGRNKYYADSVTGRFTISRDNSKNTLYL
QMNSLRAEDTAVYYCASSSGYLFHSDYWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMH
EALHNHYTQKSLSLSPGK

SEQ ID NO: 32 (C135 Fab LC)

MGWSCIILFLVATATGVHSDIQMTQSPSTLSASVGDRVTITCRASQSISN
WLAWFQQKPGKAPKLLIYEASSLESGVPSRFSGSGSGTEFTLTISSLQPD
DFATYYCQQYNSYPWTFGQGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC

SEQ ID NO: 33 (C135 HC (CH1))

MGWSCIILFLVATATGVHSQVQLVESGGGVVQPGRSLRLSCAASGFTFSS
YAMHWVRQAPGKGLEWVAVIPFDGRNKYYADSVTGRFTISRDNSKNTLYL
QMNSLRAEDTAVYYCASSSGYLFHSDYWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHT

SEQ ID NO: 34 (linker)

GGGTSGGGGS

SEQ ID NO: 35 (N(aa 44-257) - LgBiT)

MRMQLLLLIALSLALVTNSSGLPNNTASWFTALTQHGKEDLKFPRGQGVP
INTNSSPDDQIGYYRRATRRIRGGDGKMKDLSPRWYFYYLGTGPEAGLPY
GANKDGIIWVATEGALNTPKDHIGTRNPANNAAIVLQLPQGTTLPKGFYA
EGSRGGSQASSRSSSRSRNSSRNSTPGSSRGTSPARMAGNGGDAALALLL
LDRLNQLESKMSGKGQQQQGQTVTKKSAAEASKKGGGGSGGGTSGGGGSV
FTLEDFVGDWEQTAAYNLDQVLEQGGVSSLLQNLAVSVTPIQRIVRSGEN
ALKIDIHVIIPYEGLSADQMAQIEEVFKVVYPVDDHHFKVILPYGTLVID
GVTPNMLNYFGRPYEGIAVFDGKKITVTGTLWNGNKIIDERLITPDGSML
FRVTINSGGGSGHHHHHHHHHHHHGSGLNDIFEAQKIEWHEG

SEQ ID NO: 36 (N(aa 44-257) - SmBiT)

MRMQLLLLIALSLALVTNSSGLPNNTASWFTALTQHGKEDLKFPRGQGVP
INTNSSPDDQIGYYRRATRRIRGGDGKMKDLSPRWYFYYLGTGPEAGLPY
GANKDGIIWVATEGALNTPKDHIGTRNPANNAAIVLQLPQGTTLPKGFYA
EGSRGGSQASSRSSSRSRNSSRNSTPGSSRGTSPARMAGNGGDAALALLL
LDRLNQLESKMSGKGQQQQGQTVTKKSAAEASKKGGGGSGGGTSGGGGSV
TGYRLFEEILGGGSGHHHHHHHHHHHHGSGLNDIFEAQKIEWHEG

SEQ ID NO: 37 RBD UK variant smbit

TSRFPNITNLCPFGEVFNATRFASVYAWNRKRISNCVADYSVLYNSASFS
TFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQA
GSTPCNGVEGFNCYFPLQSYGFQPTYGVGYQPYRVVVLSFELLHAPATVC
GPKKSTNLGGGGSGGGGSGGGGSGGGGSGGGGSVTGYRLFEEILGGGSGH
HHHHHHHHHHH

SEQ ID NO: 38 RBD UK variant 1gbit KILYATRICKTOWNSEND 73819859 1

TSRFPNITNLCPFGEVFNATRFASVYAWNRKRISNCVADYSVLYNSASFS
TFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQA
GSTPCNGVEGFNCYFPLQSYGFQPTYGVGYQPYRVVVLSFELLHAPATVC
GPKKSTNLGGGGSGGGGSGGGGSVFTLEDFVGDWEQTAAYNLDQVLEQGG
VSSLLQNLAVSVTPIQRIVRSGENALKIDIHVIIPYEGLSADQMAQIEEV
FKVVYPVDDHHFKVILPYGTLVIDGVTPNMLNYFGRPYEGIAVFDGKKIT
VTGTLWNGNKIIDERLITPDGSMLFRVTINSGGGSGHHHHHHHHHHHH

SEQ ID NO: 39 RBD SA variant smbit

TSRFPNITNLCPFGEVFNATRFASVYAWNRKRISNCVADYSVLYNSASFS
TFKCYGVSPTKLNDLCFTNVYADSFVIRGDEVRQIAPGQTGNIADYNYKL
PDDFTGCVIAWNSNNLDSKVGGNYNYLYRLFRKSNLKPFERDISTEIYQA
GSTPCNGVKGFNCYFPLQSYGFQPTYGVGYQPYRVVVLSFELLHAPATVC
GPKKSTNLGGGGSGGGGSGGGGSGGGGSGGGGSVTGYRLFEEILGGGSGH
HHHHHHHHHHH

SEQ ID NO: 40 RBD SA variant 1gbit

TSRFPNITNLCPFGEVFNATRFASVYAWNRKRISNCVADYSVLYNSASFS
TFKCYGVSPTKLNDLCFTNVYADSFVIRGDEVRQIAPGQTGNIADYNYKL
PDDFTGCVIAWNSNNLDSKVGGNYNYLYRLFRKSNLKPFERDISTEIYQA
GSTPCNGVKGFNCYFPLQSYGFQPTYGVGYQPYRVVVLSFELLHAPATVC
GPKKSTNLGGGGSGGGGSGGGGSVFTLEDFVGDWEQTAAYNLDQVLEQGG
VSSLLQNLAVSVTPIQRIVRSGENALKIDIHVIIPYEGLSADQMAQIEEV
FKVVYPVDDHHFKVILPYGTLVIDGVTPNMLNYFGRPYEGIAVFDGKKIT
VTGTLWNGNKIIDERLITPDGSMLFRVTINSGGGSGHHHHHHHHHHHH

SEQ ID NO: 41 NTD smbit

TVNLTTRTQLPPAYTNSFTRGVYYPDKVFRSSVLHSTQDLFLPFFSNVTW
FHAIHVSGTNGTKRFDNPVLPFNDGVYFASTEKSNIIRGWIFGTTLDSKT
QSLLIVNNATNVVIKVCEFQFCNDPFLGVYYHKNNKSWMESEFRVYSSAN
NCTFEYVSQPFLMDLEGKQGNFKNLREFVFKNIDGYFKIYSKHTPINLVR
DLPQGFSALEPLVDLPIGINITRFQTLLALHRSYLTPGDSSSGWTAGAAA
YYVGYLQPRTFLLKYNENGTITDAVDGGGGSGGGGSGGGGSGGGGSGGGG
SVTGYRLFEEILGGGSGHHHHHHHHHHHH

SEQ ID NO: 42 NTD 1gbit

TVNLTTRTQLPPAYTNSFTRGVYYPDKVFRSSVLHSTQDLFLPFFSNVTW
FHAIHVSGTNGTKRFDNPVLPFNDGVYFASTEKSNIIRGWIFGTTLDSKT
QSLLIVNNATNVVIKVCEFQFCNDPFLGVYYHKNNKSWMESEFRVYSSAN
NCTFEYVSQPFLMDLEGKQGNFKNLREFVFKNIDGYFKIYSKHTPINLVR
DLPQGFSALEPLVDLPIGINITRFQTLLALHRSYLTPGDSSSGWTAGAAA
YYVGYLQPRTFLLKYNENGTITDAVDGGGGSGGGGSGGGGSVFTLEDFVG
DWEQTAAYNLDQVLEQGGVSSLLQNLAVSVTPIQRIVRSGENALKIDIHV
IIPYEGLSADQMAQIEEVFKVVYPVDDHHFKVILPYGTLVIDGVTPNMLN
YFGRPYEGIAVFDGKKITVTGTLWNGNKIIDERLITPDGSMLFRVTINSG
GGSGHHHHHHHHHHHH

SEQ ID NO: 43 NTD

TVNLTTRTQLPPAYTNSFTRGVYYPDKVFFSTQDLFLPFFSNVTWFHAIH
VSGTNGTKRFDNPVLPFNDGVYFASTEKSNIIRGWIFGTTLDSKTQSLLI
VNNATNVVIKVCEFQFCNDPFLGVYYHKNNKSWMESEFRVYSSANNCTFE
YVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGY
LQPRTFLLKYNENGTITDAVD

SEQ ID NO: 44 UK variant RBD

TSRFPNITNLCPFGEVFNATRFASVYAWNRKRISNCVADYSVLYNSASFS
TFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQA
GSTPCNGVEGFNCYFPLQSYGFQPTYGVGYQPYRVVVLSFELLHAPATVC
GPKKSTNL

SEQ ID NO: 45 SA variant RBD

TSRFPNITNLCPFGEVFNATRFASVYAWNRKRISNCVADYSVLYNSASFS
TFKCYGVSPTKLNDLCFTNVYADSFVIRGDEVRQIAPGQTGNIADYNYKL
PDDFTGCVIAWNSNNLDSKVGGNYNYLYRLFRKSNLKPFERDISTEIYQA
GSTPCNGVKGFNCYFPLQSYGFQPTYGVGYQPYRVVVLSFELLHAPATVC
GPKKSTNL

1. A method for detecting antibodies against a SARS-CoV-2 viral protein in a biological sample comprising
i) combining
a) the biological sample;
b) a first fusion protein that comprises a first SARS-CoV-2 viral protein domain and a first peptide fragment of a split reporter protein, and
c) a second fusion protein that comprises a second SARS-CoV-2 viral protein domain and a second peptide fragment of the split reporter protein
to produce a mixture;
ii) maintaining the mixture under conditions in which, only if the biological sample comprises individual antibodies, at least one of which binds the first and the second SARS-CoV-2 viral protein domain simultaneously, the first peptide fragment and the second peptide fragment associate to produce an enzymatically active reporter protein; and
iii) detecting the association of the first peptide fragment and the second peptide fragment if the biological sample comprises antibodies against the SARS-CoV-2 viral protein.

2. The method of claim 1 wherein the first and second SARS-CoV-2 viral protein domains are the same.

3. The method of claim 1, wherein the SARS-CoV-2 viral protein is the a SARS-CoV-2 N protein,
wherein the first fusion protein comprises a first SARS-CoV-2 N protein domain andthe first peptide fragment of a split reporter protein, and
the second fusion protein comprises a second SARS-CoV-2 N protein domain andthe second peptide fragment of the split reporter protein; and
wherein each of the first SARS-CoV-2 N protein domain and the second SARS-CoV-2 N protein domain comprises a sequence that is at least 90% identical to SEQ ID NO: 5.

4. The method of claim 3 wherein the first and second SARS-Cov-2 N protein domains are the same.

5. The method of claim 1, wherein the SARS-CoV-2 viral protein is a Spike protein,
wherein the first fusion protein comprises a first SARS-CoV-2 Spike RBD (SpikeRBD) domain and the first peptide fragment of a split reporter protein, and
the second fusion protein comprises a second SARS-CoV-2 SpikeRBD domain, and the second peptide fragment of the split reporter protein; and
wherein each of the first SARS-CoV-2 SpikeRBD domain and the second SARS-CoV-2 SpikeRBD domain comprises a sequence that is at least 90% identical to SEQ ID NO: 1.

6. The method of claim 5 wherein the first and second SARS-Cov-2 SpikeRBD domains are the same.

7. The method of claim 1, wherein the antibodies detected are neutralizing antibodies.

8. The method of claim 1, wherein the split reporter protein is a split-luciferase.

9. The method of claim 1, wherein the first peptide fragment of the split reporter protein comprises a sequence of SEQ ID NO: 4 (LgBiT) and second peptide fragment of the split reporter protein comprises a sequence of SEQ ID NO: 3 (SmBiT).

10. The method of claim 3, wherein the first peptide fragment of the split reporter protein is fused to the C-terminus of the SARS-CoV-2 N protein domain, and
wherein the second peptide fragment of the split reporter protein is fused to the C-terminus of the second SARS-CoV-2 N protein domain.

11. The method of claim 1, wherein the first peptide fragment of the split reporter protein is fused to the first SARS-CoV-2 viral protein domain via a first flexible linker and/or the second peptide fragment of the split reporter protein is fused to the second SARS-CoV-2 viral protein domain via a second flexible linker.

12. The method of claim 11, wherein each of the first and second flexible linkers has a length in the range of one to 50 amino acids.

13. The method of claim 5,
wherein the first peptide fragment of the split reporter protein is fused to the first SARS-CoV-2 SpikeRBD domain via a first flexible linker and/or the second peptide fragment of the split reporter protein is fused to the second SARS-CoV-2 SpikeRBD domain via a second flexible linker, and
wherein the first flexible linker has a length of 15 amino acids and the second flexible linker has a length of 25 amino acids.

14. The method of claim 3,
wherein the first peptide fragment of the split reporter protein is fused to the first SARS-CoV-2 N protein domain via a first flexible linker and/or the second peptide fragment of the split reporter protein is fused to the second SARS-CoV-2 N protein domain via a second flexible linker, and
wherein the first flexible linker and the second flexible linker has each have a length of 10 amino acids.

15. The method of claim 1, wherein the first fusion protein is present in the mixture at a concentration in the range from 0.3 nM to 10 nM, and/or the second fusion protein is present in the mixture at a concentration in the range from 0.3 nM to 10 nM.

16. The method of claim 15, wherein the first fusion protein and the second fusion protein are present in the mixture at about equal molar concentrations.

17. A kit for detecting antibodies against a SARS-CoV-2 viral protein in a biological sample, wherein the kit comprises:
i) a first fusion protein that comprises a first SARS-CoV-2 viral protein domain and a first peptide fragment of a split reporter protein, and
ii) a second fusion protein that comprises a second SARS-CoV-2 viral protein domain and a second peptide fragment of the split reporter protein,
wherein the first SARS-CoV-2 viral protein domain shares at least 90% sequence identity with the second viral protein domain.

18. The kit of claim 17, wherein
i) the first fusion protein comprises a first SARS-CoV-2 N protein domain and the first peptide fragment of a split reporter protein, and
ii) the second fusion protein comprises a second SARS-CoV-2 N protein domain and the second peptide fragment of the split reporter protein,
wherein each of the first SARS-CoV-2 N protein domain and the second SARS-CoV-2 N protein domain comprises a sequence that is at least 90% identical to SEQ ID NO: 5.

19. The kit of claim 17, wherein
i) the first fusion protein comprises a first SARS-CoV-2 SpikeRBD domain and the first peptide fragment of a split reporter protein, and
ii) the second fusion protein comprises a second SARS-CoV-2 SpikeRBD domain and the second peptide fragment of the split reporter protein,
wherein each of the first and the second SARS-CoV-2 SpikeRBD domains comprise a sequence that is at least 90% identical to SEQ ID NO: 1.

20. The kit of claim 18,
wherein the first peptide fragment of the split reporter protein is fused to the C- terminus of the first SARS-CoV-2 N protein domain, and
wherein the second peptide fragment of the split reporter protein is fused to the C-terminus of the second SARS-CoV-2 N protein domain.

21. The kit of claim 17, wherein the split reporter protein is a split-luciferase.

22. The kit of claim 17, wherein the first fusion protein and the second fusion protein are lyophilized.

23. The kit of claim 21, wherein the kit further comprises a substrate for the split-luciferase.

24. The kit of claim 17, wherein the first peptide fragment of the split reporter protein comprises a sequence of SEQ ID NO: 4 and the second peptide fragment comprise a sequence of SEQ ID NO: 3.

25. The kit of claim 17, wherein the kit further comprises a negative control sample, optionally wherein the negative control sample comprises PBST and 4-10% PBS.

26. The kit of claim 19, wherein the kit further comprises a positive control sample, wherein the positive control sample comprises an antibody known to specifically bind to the SARS-CoV-2 SpikeRBD domain or to an ACE-Fc protein.

27. The kit of claim 18, wherein the kit further comprises a positive control sample, wherein the positive control sample comprises an antibody that is known to specifically bind to the SARS-CoV-2 N protein domain.

28. A reaction mixture comprising
i) a test sample,
ii) a first fusion protein that comprises a first SARS-CoV-2 viral protein domain of a SARS-CoV-2 viral protein and a first peptide fragment of a split reporter protein, and
iii) a second fusion protein that comprises a second SARS-CoV-2 viral protein domain of the SARS-CoV-2 viral protein and a second peptide fragment of the split reporter protein,
wherein, the first peptide fragment and the second peptide fragment of the split reporter protein associate to produce a detectable reporter protein if the test sample comprises antibodies that specifically bind the first and second SARS-CoV-2 viral protein domains.

29. The reaction mixture of claim 28, wherein the SARS-CoV-2 viral protein is the Spike (S) protein, and wherein each of the first SARS-CoV-2 viral protein domain and the second SARS-CoV-2 viral protein domain comprises a sequence that is at least 90% identical to SEQ ID NO: 1.

30. The reaction mixture of claim 28, wherein the SARS-CoV-2 viral protein is the N protein, and wherein each of the first SARS-CoV-2 viral protein and the second SARS-CoV-2 viral protein comprise a sequence that is at least 90% identical to SEQ ID NO: 5.

31. A method of determining if an antibody in a test sample is competitive with a reference antibody against a SARS-CoV-2 viral protein domain, the method comprising:
i) contacting in a first reaction mixture according to claim 28 a first aliquot of the test sample and a viral protein sensor, wherein the viral protein sensor comprises the first and second fusion proteins, and detecting a first signal produced from association of the first peptide fragment and the second peptide fragment in the viral protein sensor,
ii) contacting in a second reaction mixture according to claim 28 a second aliquot of the test sample and an epitope-masked viral protein sensor, wherein the epitope-masked viral protein sensor comprises the first and second fusion proteins that are bound to a reference antibody that specifically binds to the SARS-CoV-2 viral protein domain at a known epitope, and detecting a second signal produced from association of the first peptide fragment and the second peptide fragment in the epitope-masked sensor; and
iii) determining that the test sample comprises an antibody competitive with the reference antibody if the first signal is substantially higher than the second signal.

32. The kit of claim 17, wherein the first peptide fragment and the second peptide fragment associate to produce a detectable reporter protein only if the biological sample comprises an antibody that specifically binds to both the first and second SARS-CoV-2 viral protein domains simultaneously.
